(19) United States
(12) Patent Application Publication
(10) Pub. No.: US 2024/0300940 A1
(43) Pub. Date: Sep. 12, 2024

(54) Phenothiazinyl Compounds and Uses

(71) Applicant: Prosetta Biosciences, Inc., San Francisco, CA (US)

(72) Inventors: Anatoliy Kitaygorodskyy, San Francisco, CA (US); Vishwanath R. Lingappa, San Francisco, CA (US); Shivarudraswamy Devaraju, Karnataka (IN); Dharma Prasad, Karnataka (IN)

(21) Appl. No.: 18/583,535

(22) Filed: Feb. 21, 2024

Related U.S. Application Data
(60) Provisional application No. 63/447,183, filed on Feb. 21, 2023.

(51) Int. Cl. C07D 417/14; A61K 31/5415; A61K 31/55; A61K 31/551; A61K 31/553; A61P 31/04; A61P 35/00; C07D 279/20; C07D 417/04; C07D 471/08

(52) U.S. Cl. CPC C07D 417/14; A61K 31/5415; A61K 31/55; A61K 31/551; A61K 31/553; A61P 31/04; A61P 35/00; C07D 279/20; C07D 417/04; C07D 471/08

(57) ABSTRACT

Disclosed are novel heterocyclic compounds such as novel phenothiazinyl compounds and pharmaceutical compositions thereof which may be used to treat or prevent bacterial infections including gram positive bacterial infections, skin infection, endocarditis, osteomyelitis, pneumonia, HIV infection, cancer, Alzheimer's disease, pox, rabies and coronavirus infection. Also disclosed are the general use of known phenothiazinyl compounds and pharmaceutical compositions thereof to treat r prevent bacterial infections including gram positive bacterial infections, skin infection, endocarditis, osteomyelitis, pneumonia, HIV infection, cancer, Alzheimer's disease and coronavirus infection.

### CROSS-REFERENCE TO RELATED APPLICATIONS

This application claims the benefit under 35 U.S.C. § 119(e) of U.S. Provisional Application No. 63/447,183, filed Feb. 21, 2023, which is hereby incorporated by reference in its entirety.

### FIELD

Disclosed are novel heterocyclic compounds such as novel phenothiazinyl compounds and pharmaceutical compositions thereof which may be used to treat or prevent bacterial infections including gram positive bacterial infections, skin infection, endocarditis, osteomyelitis, pneumonia, HIV infection, cancer, Alzheimer's disease, pox, rabies and coronavirus infection. Also disclosed are the general use of known phenothiazinyl compounds and pharmaceutical compositions thereof to treat r prevent bacterial infections including gram positive bacterial infections, skin infection, endocarditis, osteomyelitis, pneumonia, HIV infection, cancer, Alzheimer's disease and coronavirus infection.

### BACKGROUND

MRSA and S. aureus cause skin infection, endocarditis, osteomyelitis, and pneumonia and are becoming increasingly difficult to treat due to the rise of global antimicrobial resistance, with ever fewer therapeutic options. Efficacy of current therapies are further compromised by side effects. The paucity of new classes of antibiotics in pharma pipelines is a further cause for alarm.

Similarly, current therapies for HIV infection, cancer, Alzheimer's disease, pox, rabies and coronavirus infection are inadequate.

Accordingly, what is needed are novel classes of compounds which may be used to treat and or prevent bacterial infections, including gram positive bacterial infections, and infections caused by MRSA, MDR and S. aureus, HIV infection, cancer, Alzheimer's disease, pox, rabies and coronavirus infection.

### SUMMARY

In one aspect, a compound of Formula (I) or pharmaceutically acceptable salts, solvates or hydrates thereof is provided which satisfies these and other needs:

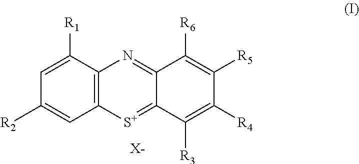

or pharmaceutically acceptable salts, solvates or hydrates thereof wherein: R₁ is H, —NR₇R₈, —OR₉, alkyl, substituted alkyl, alkenyl, substituted alkenyl, alkynyl, substituted alkynyl, heteroalkyl, substituted heteroalkyl or halo; R₂ is —H, —NR₁₀R₁₁ or

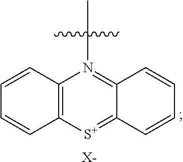

R₃ is —H, halo, —OR₁₂, alkyl or substituted alkyl; R₄ is —H, —NR₁₃R₁₄ or

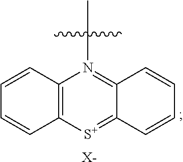

R₅ is —H or halo, —OR₁₅, alkyl or substituted alkyl; R₆ is —H, —NR₁₆R₁₇, —OR₁₈, alkyl, substituted alkyl, alkenyl, substituted alkenyl, alkynyl, substituted alkynyl, heteroalkyl, substituted heteroalkyl or halo; R₇ and R₈ are independently alkyl, substituted alkyl, alkenyl, substituted alkenyl, alkynyl, substituted alkynyl, heteroalkyl, substituted heteroalkyl or taken together with the atom to which they are bonded form a cycloheteroalkyl, substituted cycloheteroalkyl, cycloheteroalkenyl, substituted cycloheteroalkenyl, bicycloheteroalkyl, substituted bicycloheteroalkyl, spirocycloheteroalkyl or a substituted spirocycloheteroalkyl ring; R₁₀ and R₁₁ are independently alkyl, substituted alkyl, alkenyl, substituted alkenyl, alkynyl, substituted alkynyl, heteroalkyl, substituted heteroalkyl or taken together with the atom to which they are bonded form a cycloheteroalkyl, substituted cycloheteroalkyl, cycloheteroalkenyl, substituted cycloheteroalkenyl, bicycloheteroalkyl, substituted bicycloheteroalkyl, spirocycloheteroalkyl or a substituted spirocycloheteroalkyl ring; R₁₆ and R₁₇ are independently alkyl, substituted alkyl, alkenyl, substituted alkenyl, alkynyl, substituted alkynyl, heteroalkyl, substituted heteroalkyl or taken together with the atom to which they are bonded form a cycloheteroalkyl, substituted cycloheteroalkyl, cycloheteroalkenyl, substituted cycloheteroalkenyl, bicycloheteroalkyl, substituted bicycloheteroalkyl, spirocycloheteroalkyl or a substituted spirocycloheteroalkyl ring; R₉, R₁₂, R₁₅ and R₁₈ are independently alkyl, substituted alkyl, alkenyl, substituted alkenyl, alkynyl, substituted alkynyl, aryl, substituted aryl, heteroaryl, substituted aryl, heteroalkyl or substituted heteroalkyl; and X— is a pharmaceutically acceptable salt.

In some aspects, the following compounds are excluded: provided that when R₂ and R₄ both form a C₅-C₇ cycloheteroalkyl or C₅-C₇ substituted cycloheteroalkyl ring that R₁ is not H when R₆ is t-butyl, R₁ is not H or methyl when R₆ is H except when R₅ is —CH₂Boc or —OMe, R₁ is not methyl or ethyl when R₆ is ethyl; provided that none of R₁₀, R₁₁, R₁₃ or R₁₄ are alkyl or substituted alkyl when R₁ and R₆ are independently —H, alkyl, alkenyl or haloalkyl if either R₁₀ and R₁₁ or R₁₃ and R₁₄ form a C₅-C₇ cycloheteroalkyl or C₅-C₇ substituted cycloheteroalkyl ring; provided that at least one of R₁ and R₆ is not —H when one of R₂ and R₄ is —H; and provided that when R₂ is —NR₁₀R₁₁ and R₄ is —NR₁₃R₁₄ and any of R₁₀, R₁₁, R₁₃ or R₁₄ are substituted alkyl that none of R₁₀, R₁₁, R₁₃ or R₁₄ are substituted with —OR₁₉, —SR₂₀ or —NR₂₁ where R₁₉, R₂₀ or R₂₁ are independently alkyl, alkenyl or aryl. It should be understood that the above provisos may operate together or independently or not at all.

In another aspect, a compound of Formula (II) or pharmaceutically acceptable salts, solvates or hydrates thereof is provided

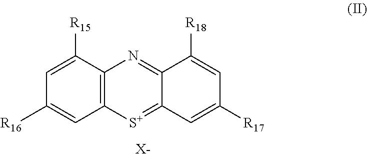

where R₁₅ is H, alkyl or halo; R₁₆ is —NR₁₉R₂₀; R₁₇ is —NR₂₁R₂₂; R₁₈ is —H, alkyl or halo; R₁₉ and R₂₀ are alkyl, or together with the atom to which they are attached form a saturated pyrrolidinyl-1-yl, saturated substituted pyrrolidinyl-1-yl, unsaturated pyrrolidinyl-1-yl, unsaturated substituted pyrrolidinyl-1-yl, saturated piperazin-1-yl, saturated substituted piperazin-1-yl, unsaturated piperazin-1-yl ring or unsaturated substituted piperazin-1-yl ring; R₂₁ and R₂₂ are alkyl, or together with the atom to which they are attached form a saturated pyrrolidinyl-1-yl, saturated substituted pyrrolidinyl-1-yl, unsaturated pyrrolidinyl-1-yl, unsaturated substituted pyrrolidinyl-1-yl, saturated piperazin-1-yl, saturated substituted piperazin-1-yl, unsaturated piperazin-1-yl ring or unsaturated substituted piperazin-1-yl ring; X— is a pharmaceutically acceptable salt; provided that the compound of Formula (II) is not

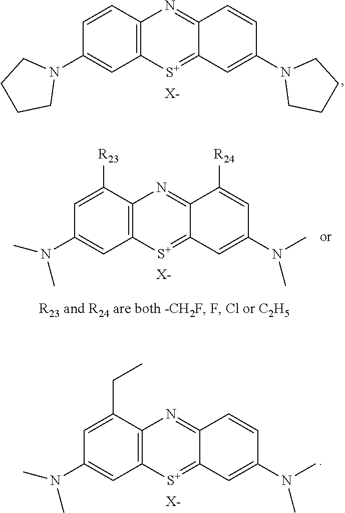

Also provided are derivatives, including salts, esters, enol ethers, enol esters, solvates, hydrates, metabolites and prodrugs of the compounds described herein. Further provided are pharmaceutical compositions which include the compounds provided herein and a pharmaceutically acceptable vehicle.

In still another aspect, methods of treating, preventing, or ameliorating symptoms of medical disorders such as, for example, bacterial infections including gram positive bacterial infections, skin infection, endocarditis, osteomyelitis, pneumonia, HIV infection, cancer, Alzheimer's disease, pox, rabies and coronavirus infection which comprise administering to a patient in need thereof therapeutically acceptable amount of a compound of structural Formula (I) or a pharmaceutically acceptable composition thereof are provided.

In some aspect aspects, when treating or preventing cancer the patient is mammal which is not a member of the species Homo sapiens.

### BRIEF DESCRIPTION OF THE DRAWINGS

FIG. 1 illustrates time kill kinetics of compound 1 against MSSA ATCC25923.

FIG. 2 illustrates time kill kinetics of compound 1 against MSSA ATCC43300.

FIG. 3 illustrates time kill kinetics of compound 1 against MDR S. aureus.

FIG. 4 illustrates post antibiotic effect of compound 1 against MRSA.

FIG. 5 illustrates post antibiotic effect of compound 1 against MDR S. aureus.

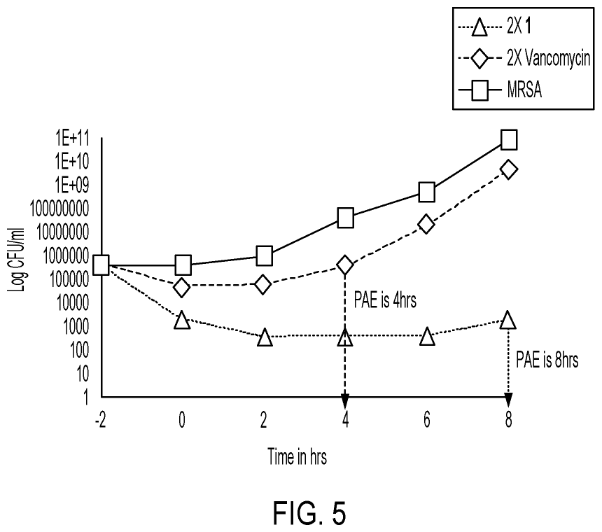

FIG. 6 illustrates biofilm eradication against MRSA by compound 1.

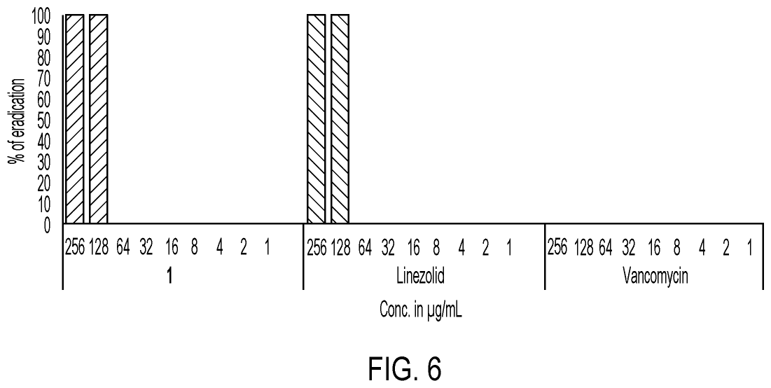

FIG. 7 illustrates the mean serum concentration time profiles after single intravenous dose of compound 1 in mice.

FIG. 8 illustrates tumor volumes time when treated with a control, gemcitabine and compound 68.

### DETAILED DESCRIPTION

### Definitions

Unless defined otherwise, all technical and scientific terms used herein have the same meaning as is commonly understood by one of ordinary skill in the art to which this invention belongs. If a plurality of definitions for a term exist herein, those in this section prevail unless stated otherwise.

As used herein, and unless otherwise specified, the terms “about” and “approximately,” when used in connection with a property with a numeric value or range of values indicate that the value or range of values may deviate to an extent deemed reasonable to one of ordinary skill in the art while still describing the particular property. Specifically, the terms “about” and “approximately,” when used in this context, indicate that the numeric value or range of values may vary by 5%, 4%, 3%, 2%, 1%, 0.9%, 0.8%, 0.7%, 0.6%, 0.5%, 0.4%, 0.3%, 0.2% or 0.1% of the recited value or range of values. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

A dash (“—”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “C_{u,v}” indicates that the following group has from u to v carbon atoms. It should be understood that u to v carbons includes u+1 to v, u+2 to v, u+3 to v, etc. carbons, u+1 to u+3 to v, u+1 to u+4 to v, u+2 to u+4 to v, etc. and cover all possible permutation of u and v.

“Alkyl,” by itself or as part of another substituent, refers to a saturated, branched, or straight-chain monovalent hydrocarbon radical derived by the removal of one hydrogen atom from a single carbon atom of a parent alkane. Typical alkyl groups include, but are not limited to, methyl; ethyl; propyls such as propan-1-yl, propan-2-yl, etc.; butyls such as butan-1-yl, butan-2-yl, 2-methyl-propan-1-yl, 2-methyl-propan-2-yl, etc.; and the like. In some aspects, an alkyl group comprises from 1 to 20 carbon atoms (C₁-C₂₀ alkyl). In other aspects, an alkyl group comprises from 1 to 10 carbon atoms (C₁-C₁₀ alkyl). In still other aspects, an alkyl group comprises from 1 to 6 carbon atoms (C₁-C₆ alkyl).

“Alkenyl,” by itself or as part of another substituent, refers to an unsaturated branched, straight-chain having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, etc.; and the like. In some aspects, an alkenyl group comprises from 2 to 20 carbon atoms (C₂-C₂₀ alkenyl). In other aspects, an alkenyl group comprises from 2 to 10 carbon atoms (C₂-C₁₀ alkenyl). In still other aspects, an alkenyl group comprises from 2 to 6 carbon atoms (C₂-C₆ alkenyl).

“Alkynyl,” by itself or as part of another substituent refers to an unsaturated branched, straight-chain having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. In some aspects, an alkynyl group comprises from 2 to 20 carbon atoms (C₂-C₂₀ alkynyl). In other aspects, an alkynyl group comprises from 2 to 10 carbon atoms (C₂-C₁₀ alkynyl). In still other aspects, an alkynyl group comprises from 2 to 6 carbon atoms (C₂-C₆ alkynyl).

“Aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene and the like. In some aspects, an aryl group comprises from 6 to 30 carbon atoms (C₆-C₃₀ aryl). In other aspects, an aryl group comprises from 6 to 20 carbon atoms (C₆-C₂₀ aryl). In still other aspects, an aryl group comprises from 6 to 15 carbon atoms (C₆-C₁₅ aryl). In still other aspects, an aryl group comprises from 6 to 10 carbon atoms (C₆-C₁₀ aryl).

“Arylalkyl,” by itself or as part of another substituent, refers to an acyclic alkyl group in which one of the hydrogen atoms bonded to a carbon atom, typically a terminal or sp³ carbon atom, is replaced with an aryl group as, as defined herein. Typical arylalkyl groups include, but are not limited to, benzyl, 2-phenylethan-1-yl, 1-phenylethen-1-yl, naphthylmethyl, 2-naphthylethan-1-yl, 1-naphthylethen-1-yl, naphthobenzyl, 2-naphthophenylethan-1-yl and the like. In some aspects, an arylalkyl group is (C₇-C₄₀) arylalkyl, e.g., the alkyl moiety of the arylalkyl group is (C₁-C₁₀) alkyl and the aryl moiety is (C₆-C₃₀) aryl. In other aspects, an arylalkyl group is (C₇-C₃₀) arylalkyl, e.g., the alkyl moiety of the arylalkyl group is (C₁-C₁₀) alkyl and the aryl moiety is (C₆-C₂₀) aryl. In other aspects, an arylalkyl group is (C₇-C₂₀) arylalkyl, e.g., the alkyl moiety of the arylalkyl group is (C₁-C₅) alkyl and the aryl moiety is (C₆-C₁₂) aryl. In still other aspects, an arylalkyl group is (C₇-C₁₅) arylalkyl, e.g., the alkyl moiety of the arylalkyl group is (C₁-C₅) alkyl and the aryl moiety is (C₆-C₁₀) aryl.

“Arylalkenyl,” by itself or as part of another substituent, refers to an acyclic alkenyl group in which one of the hydrogen atoms bonded to a carbon atom, is replaced with an aryl group as, as defined herein. In some aspects, an arylalkenyl group is (C₈-C₄₀) arylalkenyl, e.g., the alkenyl moiety of the arylalkenyl group is (C₂-C₁₀) alkenyl and the aryl moiety is (C₆-C₃₀) aryl. In other aspects, an arylalkenyl group is (C₈-C₃₀) arylalkenyl, e.g., the alkenyl moiety of the arylalkenyl group is (C₂-C₁₀) alkenyl and the aryl moiety is (C₈-C₂₀) aryl. In other aspects, an arylalkenyl group is (C₈-C₂₀) arylalkenyl, e.g., the alkenyl moiety of the arylalkenyl group is (C₂-C₈) alkenyl and the aryl moiety is (C₆-C₁₂) aryl. In still other aspects, an arylalkenyl group is (C₈-C₁₅) arylalkenyl, e.g., the alkenyl moiety of the arylalkenyl group is (C₂-C₅) alkenyl and the aryl moiety is (C₆-C₁₀) aryl.

“Arylalkynyl,” by itself or as part of another substituent, refers to an acyclic alkynyl group in which one of the hydrogen atoms bonded to a carbon atom, is replaced with an aryl group as, as defined herein. In some aspects, an arylalkynyl group is (C₈-C₄₀) arylalkynyl, e.g., the alkynyl moiety of the arylalkynyl group is (C₂-C₁₀) alkynyl and the aryl moiety is (C₆-C₃₀) aryl. In other aspects, an arylalkynyl group is (C₈-C₃₀) arylalkynyl, e.g., the alkynyl moiety of the arylalkynyl group is (C₂-C₁₀) alkynyl and the aryl moiety is (C₆-C₂₀) aryl. In other aspects, an arylalkynyl group is (C₈-C₂₀) arylalkynyl, e.g., the alkynyl moiety of the arylalkenyl group is (C₂-C₅) alkynyl and the aryl moiety is (C₆-C₁₂) aryl. In still other aspects, an arylalkynyl group is (C₈-C₁₅) arylalkynyl, e.g., the alkynyl moiety of the arylalkynyl group is (C₂-C₅) alkynyl and the aryl moiety is (C₆-C₁₀) aryl.

Bicycloheteroalkyl,” by itself or as part of another substituent refers to a double ring alkyl structure which shares two atoms and which comprise at least one hetero atom independently selected from the group consisting of N, O, and S in the ring.

“Compounds,” refers to compounds encompassed by structural formulae disclosed herein and includes any specific compounds within these formulae whose structure is disclosed herein. Compounds may be identified either by their chemical structure and/or chemical name. The chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass the stereoisomerically pure form depicted in the structure (e.g., geometrically pure, enantiomerically pure or diastereomerically pure). The chemical structures depicted herein also encompass the enantiomeric and stereoisomeric derivatives of the compound depicted. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds may also be atropisomers. The compounds described also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms. In general, compounds may be hydrated or solvated. Certain compounds may exist in multiple crystalline or amorphous forms. In general, all physical forms are equivalent for the uses contemplated herein and are intended to be within the scope of the present disclosure. Further, it should be understood, when partial structures of the compounds are illustrated, that brackets indicate the point of attachment of the partial structure to the rest of the molecule.

“Cycloalkyl,” by itself or as part of another substituent, refers to a saturated cyclic monovalent hydrocarbon radical derived by the removal of one hydrogen atom from a single carbon atom of a parent cycloalkane. Typical cycloalkyl groups include, but are not limited to, cyclopropyl, cyclobutyl, cyclopentyl cyclopentenyl; etc.; and the like. In some aspects, a cycloalkyl group comprises from 3 to 20 carbon atoms (C₃-C₁₅ cycloalkyl). In other aspects, a cycloalkyl group comprises from 3 to 10 carbon atoms (C₃-C₁₀ cycloalkyl). In still other aspects, a cycloalkyl group comprises from 3 to 8 carbon atoms (C₃-C₈ cycloalkyl). The term “cyclic monovalent hydrocarbon radical” also includes multicyclic hydrocarbon ring systems having a single radical and between 5 and 12 carbon atoms. Exemplary multicyclic cycloalkyl rings include, for example, norbornyl, pinyl, and adamantyl.

“Cycloalkenyl,” by itself or as part of another substituent, refers to an unsaturated cyclic monovalent hydrocarbon radical derived by the removal of one hydrogen atom from a single carbon atom of a parent cycloalkene. Typical cycloalkenyl groups include, but are not limited to, cyclopropenyl, cyclobutenyl, cyclopentenyl; etc.; and the like. In some aspects, a cycloalkenyl group comprises from 3 to 20 carbon atoms (C₃-C₂₀ cycloalkenyl). In other aspects, a cycloalkenyl group comprises from 3 to 10 carbon atoms (C₃-C₁₀ cycloalkenyl). In still other aspects, a cycloalkenyl group comprises from 3 to 8 carbon atoms (C₃-C₈ cycloalkenyl).

“Cycloheteroalkyl,” by itself or as part of another substituent, refers to a cycloalkyl group as defined herein in which one or more of the carbon atoms (and optionally any associated hydrogen atoms), are each, independently of one another, replaced with the same or different heteroatoms or heteroatomic groups as defined in “heteroalkyl” below. In some aspects, a cycloheteroalkyl group comprises from 3 to 20 carbon and hetero atoms (₃₋₂₀cycloheteroalkyl). In other aspects, a cycloheteroalkyl group comprises from 3 to 10 carbon and hetero atoms (₃₋₁₀ cycloheteroalkyl). In still other aspects, a cycloheteroalkyl group comprises from 3 to 8 carbon and hetero atoms (₃₋₈cycloheteroalkyl). The term “cyclic monovalent heteroalkyl radical” also includes multicyclic heteroalkyl ring systems having a single radical and between 3 and 12 carbon and at least one hetero atom. Exemplary cycloheteroalkyl groups include, for example, azetidine, pyrrolidine, piperazine, piperidine, morpholine and tetrahydrofuran.

“Cycloheteroalkenyl,” by itself or as part of another substituent, refers to a cycloalkenyl group as defined herein in which one or more of the carbon atoms (and optionally any associated hydrogen atoms), are each, independently of one another, replaced with the same or different heteroatoms or heteroatomic groups as defined in “heteroalkenyl” below. In some aspects, a cycloheteroalkenyl group comprises from 3 to 20 carbon and hetero atoms (₃₋₂₀ cycloheteroalkenyl). In other aspects, a cycloheteroalkenyl group comprises from 3 to 10 carbon and hetero atoms (₃₋₁₀₎cycloheteroalkenyl). In still other aspects, a cycloheteroalkenyl group comprises from 3 to 8 carbon and heteroatoms (₃₋₈cycloheteroalkenyl). The term “cyclic monovalent heteroalkenyl radical” also includes multicyclic heteroalkenyl ring systems having a single radical and between 2 and 12 carbon and at least one hetero atom.

“Halo,” by itself or as part of another substituent refers to a radical —F, —Cl, —Br or —I.

“Heteroalkyl,” refer to an alkyl, group, in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, —O—, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR⁵⁰¹R⁵⁰², ═N—N═, —N═N—, —N═N—NR⁵⁰³R⁴⁰⁴, —PR⁵⁰⁵—, —P(O)₂—, —POR⁵⁰⁶—, —O—P(O)₂—, —SO—, —SO₂—, —SnR⁵⁰⁷R⁵⁰⁸ and the like, where R⁵⁰¹, R⁵⁰², R⁵⁰³, R⁵⁰⁴, R⁵⁰⁵, R⁵⁰⁶, R⁵⁰⁷ and R⁵⁰⁸ are independently hydrogen, alkyl, aryl, substituted aryl, heteroalkyl, heteroaryl or substituted heteroaryl. In some aspects, an heteroalkyl group comprises from 1 to 20 carbon and hetero atoms (₁₋₂₀ heteroalkyl). In other aspects, an heteroalkyl group comprises from 1 to 10 carbon and hetero atoms (₁₋₁₀heteroalkyl). In still other aspects, an heteroalkyl group comprises from 1 to 6 carbon and hetero atoms (₁₋₆ heteroalkyl).

“Heteroalkenyl,” refers to an alkenyl group in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, —O—, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR⁵⁰¹R⁵⁰² ═N—N═, —N═N—, —N═N—NR⁵⁰³R⁴⁰⁴, —PR⁵⁰⁵—, —P(O)₂—, —POR⁰⁶—, —O—P(O)₂—, —SO—, —SO₂—, —SnR⁵⁰⁷R⁵⁰⁸ and the like, where R⁵⁰¹, R⁵⁰², R⁵⁰³, R⁵⁰⁴, R⁵⁰⁵, R⁵⁰⁶, R⁵⁰⁷ and R⁵⁰⁸ are independently hydrogen, alkyl, aryl, substituted aryl, heteroalkyl, heteroaryl or substituted heteroaryl. In some aspects, an heteroalkenyl group comprises from 1 to 20 carbon and hetero atoms (₁₋₂₀ heteroalkenyl). In other aspects, an heteroalkenyl group comprises from 1 to 10 carbon and hetero atoms (₁₋₁₀ heteroalkenyl). In still other aspects, an heteroalkenyl group comprises from 1 to 6 carbon and hetero atoms (₁₋₆ heteroalkenyl).

“Heteroaryl,” by itself or as part of another substituent, refers to a monovalent heteroaromatic radical derived by the removal of one hydrogen atom from a single atom of a parent heteroaromatic ring system, as defined herein. Typical heteroaryl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some aspects, the heteroaryl group comprises from 5 to 20 ring atoms (5-20 membered heteroaryl). In other aspects, the heteroaryl group comprises from 5 to 10 ring atoms (5-10 membered heteroaryl). Exemplary heteroaryl groups include those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyl,” by itself or as part of another substituent refers to an acyclic alkyl group in which one of the hydrogen atoms bonded to a carbon atom, typically a terminal or sp³ carbon atom, is replaced with a heteroaryl group. In some aspects, the heteroarylalkyl group is a 6-21 membered heteroarylalkyl, e.g., the alkyl moiety of the heteroarylalkyl is (C₁-C₆) alkyl and the heteroaryl moiety is a 5-15-membered heteroaryl. In other aspects, the heteroarylalkyl is a 6-13 membered heteroarylalkyl, e.g., the alkyl moiety is (C₁-C₃) alkyl and the heteroaryl moiety is a 5-10 membered heteroaryl.

“Heteroarylalkenyl,” by itself or as part of another substituent refers to an acyclic alkenyl group in which one of the hydrogen atoms bonded to a carbon atom, is replaced with a heteroaryl group. In some aspects, the heteroarylalkenyl group is a 7-21 membered heteroarylalkenyl, e.g., the alkenyl moiety of the heteroarylalkenyl is (C₂-C₆) alkenyl and the heteroaryl moiety is a 5-15-membered heteroaryl. In other aspects, the heteroarylalkenyl is a 7-13 membered heteroarylalkenyl, e.g., the alkenyl moiety is (C₂-C₃) alkenyl and the heteroaryl moiety is a 5-10 membered heteroaryl.

“Heteroarylalkynyl,” by itself or as part of another substituent refers to an acyclic alkenyl group in which one of the hydrogen atoms bonded to a carbon atom, is replaced with a heteroaryl group. In some aspects, the heteroarylalkynyl group is a 7-21 membered heteroarylalkynyl, e.g., the alkynyl moiety of the heteroarylalkynyl is (C₂-C₆) alkynyl and the heteroaryl moiety is a 5-15-membered heteroaryl. In other aspects, the heteroarylalkynyl is a 7-13 membered heteroarylalkynyl, e.g., the alkynyl moiety is (C₂-C₃) alkynyl and the heteroaryl moiety is a 5-10 membered heteroaryl.

“Hydrates,” refers to incorporation of water into to the form of a compound described herein, in stoichiometric proportions, resulting in the formation of an adduct. Methods of making hydrates include, but are not limited to, storage in an atmosphere containing water vapor, dosage forms that include water, or routine pharmaceutical processing steps such as, for example, crystallization (i.e., from water or mixed aqueous solvents), lyophilization, wet granulation, aqueous film coating, or spray drying. Hydrates may also be formed, under certain circumstances, from crystalline solvates upon exposure to water vapor, or upon suspension of the anhydrous material in water. Hydrates may also crystallize in more than one form resulting in hydrate polymorphism. See e.g., (Guillory, K., Chapter 5, pp. 202-205 in Polymorphism in Pharmaceutical Solids, (Brittain, H. ed.), Marcel Dekker, Inc., New York, NY, 1999). The above methods for preparing hydrates are well within the ambit of those of skill in the art, are completely conventional and do not require any experimentation beyond what is typical in the art. Hydrates may be characterized and/or analyzed by methods well known to those of skill in the art such as, for example, single crystal X-ray diffraction, X-ray powder diffraction, polarizing optical microscopy, thermal microscopy, thermogravimetry, differential thermal analysis, differential scanning calorimetry, IR spectroscopy, Raman spectroscopy and NMR spectroscopy. (Brittain, H., Chapter 6, pp. 205-208 in Polymorphism in Pharmaceutical Solids, (Brittain, H. ed.), Marcel Dekker, Inc. New York, 1999). In addition, many commercial companies routinely offer services that include preparation and/or characterization of hydrates such as, for example, HOLODIAG, Pharmaparc II, Voie de l'Innovation, 27 100 Val de Reuil, France (http://www.holodiag.com).

“N-oxide,” refers to a compound containing an N—O bond with three additional hydrogen or side chains attached to the N, or a compound containing an N—O bond with two additional hydrogen or side chains attached to the N, so that there is a positive charge on the nitrogen. The N-oxides of the present disclosure can be synthesized by oxidation procedures well known to those skilled in the art.

“Parent Aromatic Ring System,” refers to an unsaturated cyclic or polycyclic ring system having a conjugated pi electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System,” refers to a parent aromatic ring system in which one or more carbon atoms (and optionally any associated hydrogen atoms) are each independently replaced with the same or different heteroatom. Typical heteroatoms to replace the carbon atoms include, but are not limited to, N, P, O, S, Si, etc. Specifically included within the definition of “parent heteroaromatic ring system” are fused ring systems in which one or more of the rings are aromatic and one or more of the rings are saturated or unsaturated, such as, for example, benzodioxan, benzofuran, chromane, chromene, indole, indoline, xanthene, etc. Typical parent heteroaromatic ring systems include, but are not limited to, arsindole, carbazole, b-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Pharmaceutically acceptable salt,” refers to a salt of a compound which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Preventing,” or “prevention,” refers to a reduction in risk of acquiring a disease or disorder (i.e., causing at least one of the clinical symptoms of the disease not to develop in a patient that may be exposed to or predisposed to the disease but does not yet experience or display symptoms of the disease). The application of a therapeutic for preventing or prevention of a disease or disorder is known as ‘prophylaxis.’ In some aspects, the compounds provided herein provide superior prophylaxis because of lower long term side effects over long time periods.

“Prodrug,” as used herein, refers to a derivative of a drug molecule that requires a transformation within the body to release the active drug. Prodrugs are frequently, although not necessarily, pharmacologically inactive until converted to the parent drug.

“Promoiety,” as used herein, refers to a form of protecting group that when used to mask a functional group within a drug molecule converts the drug into a prodrug. Typically, the promoiety will be attached to the drug via bond(s) that are cleaved by enzymatic or non-enzymatic means in vivo.

“Protecting group,” refers to a grouping of atoms that when attached to a reactive functional group in a molecule masks, reduces or prevents reactivity of the functional group during chemical synthesis. Examples of protecting groups can be found in Green et al., “Protective Groups in Organic Chemistry,” (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

Spirocycloheteroalkyl,” by itself or as part of another substituent refers to a double ring alkyl structure which shares one atom and which comprise at least one hetero atom independently selected from the group consisting of N, O, and S in the ring.

“Solvates,” refers to incorporation of solvents into to the crystal lattice of a compound described herein, in stoichiometric proportions, resulting in the formation of an adduct. Methods of making solvates include, but are not limited to, storage in an atmosphere containing a solvent, dosage forms that include the solvent, or routine pharmaceutical processing steps such as, for example, crystallization (i.e., from solvent or mixed solvents) vapor diffusion, etc. Solvates may also be formed, under certain circumstances, from other crystalline solvates or hydrates upon exposure to the solvent or upon suspension material in solvent. Solvates may crystallize in more than one form resulting in solvate polymorphism. See e.g., (Guillory, K., Chapter 5, pp. 202-205 in Polymorphism in Pharmaceutical Solids, (Brittain, H. ed.), Marcel Dekker, Inc., New York, NY, 1999)). The above methods for preparing solvates are well within the ambit of those of skill in the art, are completely conventional and do not require any experimentation beyond what is typical in the art. Solvates may be characterized and/or analyzed by methods well known to those of skill in the art such as, for example, single crystal X-ray diffraction, X-ray powder diffraction, polarizing optical microscopy, thermal microscopy, thermogravimetry, differential thermal analysis, differential scanning calorimetry, IR spectroscopy, Raman spectroscopy and NMR spectroscopy. (Brittain, H., Chapter 6, pp. 205-208 in Polymorphism in Pharmaceutical Solids, (Brittain, H. ed.), Marcel Dekker, Inc. New York, 1999). In addition, many commercial companies routinely offer services that include preparation and/or characterization of solvates such as, for example, HOLODIAG, Pharmaparc II, Voie de l'Innovation, 27 100 Val de Reuil, France (http://www.holodiag.com).

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include R^{a}, halo, —O—, ═O, —OR^{b}, —SR^{b}, —S—, ═S, —NR^{c}R^{c}, ═NR^{b}, ═N—OR^{b}, trihalomethyl, —CF₃, —CN, —OCN, —SCN, —NO, —NO₂, —N—OR^{b}, —N—NR^{c}R^{c}, —NR^{b}S(O)₂R^{b}, ═N₂, —N₃, —S(O)₂R^{b}, —S(O)₂NR^{b}R^{b}, —S(O)₂O⁻, —S(O)₂OR^{b}, —OS(O)₂R^{b}, —OS(O)₂O⁻, —OS(O)₂OR^{b}, —OS(O)₂NR^{c}NR^{c}, —P(O)(O⁻)₂, —P(O)(OR^{b})(O⁻), —P(O)(OR^{b})(OR^{b}), —C(O)R^{b}, —C(O)NR^{b}—OR^{b}—C(S) R^{b}, —C(NR^{b})R^{b}, —C(O)O—, —C(O)OR^{b}, —C(S)OR^{b}, —C(O)NR^{c}R^{c}, —C(NR^{b})NR^{c}R^{c}, —OC(O)R^{b}, —OC(S) R^{b}, —OC(O)O—, —OC(O)OR^{b}, —OC(O)NR^{c}R^{c}, —OC(NCN)NR^{c}R^{c}—OC(S)OR^{b}, —NR^{b}C(O)R^{b}, —NR^{b}C(S)R^{b}, —NR^{b}C(O)O—, —NR^{b}C(O)OR^{b}, —NR^{b}C(NCN)OR^{b}, —NR^{b}S(O)₂NR^{c}R^{c}, —NR^{b}C(S)OR^{b}, —NR^{b}C(O)NR^{c}R^{c}, —NR^{b}C(S)NR^{c}R^{c}, —NR^{b}C(S)NR^{b}C(O)R^{a}, —NR^{b}S(O)₂OR^{b}, —NR^{b}S(O)₂R^{b}, —NR^{b}C(NCN) NR^{c}R^{c}, —NR^{b}C(NR^{b})R^{b} and —NR^{b}C(NR^{b})NR^{c}R^{c}, where each R^{a} is independently, substituted alkyl, substituted alkenyl, substituted alkynyl, aryl, substituted aryl, cycloalkyl, substituted cycloalkyl, cycloalkenyl, substituted cycloalkenyl, heteroalkyl, substituted heteroalkyl, heteroalkenyl, substituted heteroalkenyl, heteroalkynyl, substituted heteroalkynyl, heteroaryl or substituted heteroaryl; each R^{b} is independently hydrogen, substituted alkyl, substituted alkenyl, substituted alkynyl, cycloalkyl, substituted cycloalkyl, cycloalkenyl, substituted cycloalkenyl, heteroalkyl, substituted heteroalkyl, heteroalkenyl, substituted heteroalkenyl, heteroalkynyl, substituted heteroalkynyl, arylalkyl, substituted arylalkyl, arylalkenyl, substituted arylalkenyl, arylalkynyl, substituted arylalkynyl, heteroarylalkyl, substituted heteroarylalkyl, heteroarylalkenyl, substituted heteroarylalkenyl, heteroarylalkynyl or substituted heteroarylalkynyl; and each R^{c} is independently R^{b} or alternatively, the two R's taken together with the nitrogen atom to which they are bonded form a 4-, 5-, 6- or 7 membered-cycloheteroalkyl, substituted cycloheteroalkyl, cycloheteroalkenyl, substituted cycloheteroalkenyl ring or a cycloheteroalkyl or cycloheteroalkenyl fused with an aryl group which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl. In other aspects, substituent groups useful for substituting saturated carbon atoms in the specified group or radical include R^{a}, halo, —OR^{b}, —NR^{c}R^{c},

trihalomethyl, —CN, —NR^{b}S(O)₂R^{b}, —C(O)R^{b}, —C(O)NR^{b}—OR^{b}, —C(O)OR^{b}, —C(O)NR^{c}R^{c}, —OC(O)R^{b}, —OC(O)OR^{b}, —OS(O)₂NR^{c}NR^{c}, —OC(O)NR^{c}R^{c}, and —NR^{b}C(O)OR^{b}, where R^{a}, R^{b} and R^{c} are as previously defined. In still other aspects, substituent groups useful for substituting saturated carbon atoms in the specified group or radical include R^{a}, halo, —OR^{b}, —NR^{c}R^{c}, trihalomethyl, —CN, —C(O)R^{b}, —C(O)OR^{b}, —C(O)NR^{c}R^{c}, —OC(O)R^{b}, —OC(O)NR^{c}R^{c}, and —NR^{b}C(O)OR^{b}, where R^{a}, R^{b} and R^{c} are as previously defined.

Substituent groups useful for substituting unsaturated carbon atoms in the specified group or radical include substituted alkyl, —R^{a}, halo, —O—, —OR^{b}, —SR^{b}, —S—, —NR^{c}R^{c}, trihalomethyl, —CF₃, —CN, —OCN, —SCN, —NO, —NO₂, —N₃, —S(O)₂O—, —S(O)₂OR^{b}, —OS(O)₂R^{b}, —OS(O)₂OR^{b}, —OS(O)₂O⁻, —P(O)(O⁻)₂, —P(O)(OR^{b})(O⁻), —P(O)(OR^{b})(OR^{b}), —C(O)R^{b}, —C(S)R^{b}, —C(NR^{b}) R^{b}, —C(O)O—, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O) O⁻, —OC(O)OR^{b}, —OC(S)OR^{b}, —OC(O)NR^{c}R^{c}, —OS(O)₂NR^{c}NR^{e}, —NR^{b}C(O)R^{b}, —NR^{b}C(S)R^{b}, —NR^{b}C(O)O—, —NR^{b}C(O)OR^{b}, —NR^{b}S(O)₂OR^{a}, —NR^{b}S(O)₂R^{a}, —NR^{b}C(S)OR^{b}, —NR^{b}C(O)NR^{c}R^{c}, —NR^{b}C(NR^{b})R^{b}, —NR^{b}C(NR^{b})NR^{c}R^{c} and —C(NR^{b})NR^{b}C(NR^{b})NR^{c}R^{c} where R^{a}, R^{b} and R^{c} are as previously defined. In other aspects, substituent groups useful for substituting unsaturated carbon atoms in the specified group or radical include substituted alkyl, —R^{a}, halo, —OR^{b}, —SR^{b}, —NR—R—, trihalomethyl, —CN, —S(O)₂OR^{b}, —C(O)R^{b}, —C(O)OR^{b}, —C(O)NR^{c}R^{c}, —OC(O)R^{b}, —OC(O)OR^{b}, —OS (O)₂NR^{c}NR^{c}, —NR^{b}C(O)R^{b} and —NR^{b}C(O)OR^{b}, where R^{a}, R^{b} and R^{c} are as previously defined. In still other aspects, substituent groups useful for substituting unsaturated carbon atoms in the specified group or radical include substituted alkyl, —R^{a}, halo, —OR^{b}, —NR^{c}R^{c}, trihalomethyl, —S(O)₂OR^{b}, —C(O)R^{b}, —C(O)OR^{b}, —C(O)NR^{c}R^{c}, —OC(O)R^{b}, —NR^{b}C(O)R^{b} and —NR^{b}C(O)OR^{b}, where R^{a}, R^{b} and R^{c} are as previously defined.

Substituent groups useful for substituting nitrogen atoms in heteroalkyl and cycloheteroalkyl groups include, but are not limited to, alkyl, —R^{a}, —O⁻, —OR^{b}, —SR^{b}, —S—, —NR^{c}R^{c}, trihalomethyl, —CF₃, —CN, —NO, —NO₂, —S(O)₂R^{b}, —S(O)₂O—, —S(O)₂OR^{b}, —OS(O)₂R^{b}, —OS(O)₂O—, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b}, —NR^{b}C(NR^{b})NR^{c}R^{c} and —C(NR^{b})NR^{b}C(NR^{b})NR^{c}R^{c} where R^{a}, R^{b} and R^{c} are as previously defined. In some aspects, substituent groups useful for substituting nitrogen atoms in heteroalkyl and cycloheteroalkyl groups include, alkyl, R^{a}, halo, —OR^{b}, —NR—R—, trihalomethyl, —CN, —S(O)₂OR^{b}, —OS(O)₂R^{b}, —C(O)R^{b}, —C(NR^{b})R^{b}, —C(O)OR^{b}, —C(O)NR^{c}R^{c}, —OC(O)R^{b}, —OC(O)OR^{b}, —OS(O)₂NR^{c}NR^{c}, —NR^{b}C(O)R^{b} and —NR^{b}C(O)OR^{b}, where R^{a}, R^{b} and R^{c} are as previously defined. In still other aspects, substituent groups useful for substituting nitrogen atoms in heteroalkyl and cycloheteroalkyl groups include, alkyl, R^{a}, halo, —OR^{b}, —NR^{c}R^{c}, trihalomethyl, —CN, —S(O)₂OR^{b}, —C(O)R^{b}, —C(NR^{b})R^{b}, —C(O)OR^{b}, —C(O)NR^{c}R^{c}, —OC(O)R^{b}, —NR^{b}C(O)R^{b} and —NR^{b}C(O)OR^{b}, where R^{a}, R^{b} and R^{c} are as previously defined.

Substituent groups from the above lists useful for substituting other specified groups or atoms will be apparent to those of skill in the art.

The substituents used to substitute a specified group can be further substituted, typically with one or more of the same or different groups selected from the various groups specified above.

“Subject,” “individual,” or “patient,” is used interchangeably herein and refers to a vertebrate, preferably a mammal. Mammals include, but are not limited to, rodents, simians, humans, farm animals, sport animals and pets. In some aspects, the subject, individual, or patient is a member of the species Homo sapiens. In other aspects, the subject, individual, or patient includes all mammals except Homo sapiens.

“Treating,” or “treatment,” of any disease or disorder refers, in some aspects, to ameliorating the disease or disorder (i.e., arresting or reducing the development of the disease or at least one of the clinical symptoms thereof). Treatment may also be considered to include preemptive or prophylactic administration to ameliorate, arrest or prevent the development of the disease or at least one of the clinical symptoms. In a further feature the treatment rendered has lower potential for long-term side effects over multiple years. In other aspects “treating” or “treatment” refers to ameliorating at least one physical parameter, which may not be discernible by the patient. In yet other aspects, “treating” or “treatment” refers to inhibiting the disease or disorder, either physically (e.g., stabilization of a discernible symptom), physiologically (e.g., stabilization of a physical parameter) or both. In yet other aspects, “treating” or “treatment” refers to delaying the onset of the disease or disorder.

“Therapeutically effective amount,” means the amount of a compound that, when administered to a patient for treating a disease, is sufficient to treat the disease. The “therapeutically effective amount” will vary depending on the compound, the disease and its severity and the age, weight, adsorption, distribution, metabolism and excretion etc., of the patient to be treated.

“Vehicle,” refers to a diluent, excipient or carrier with which a compound is administered to a subject. In some aspects, the vehicle is pharmaceutically acceptable.

### Compounds

In some aspects, a compound of Formula (I) is provided:

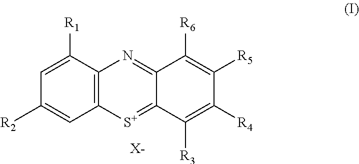

or pharmaceutically acceptable salts, solvates or hydrates thereof wherein: R₁ is H, —NR₇R₈, —OR₉, alkyl, substituted alkyl, alkenyl, substituted alkenyl, alkynyl, substituted alkynyl, heteroalkyl, substituted heteroalkyl or halo; R₂ is —H, —NR₁₀R₁₁ or

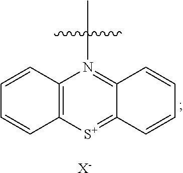

R₃ is —H, halo, —OR₁₂, alkyl or substituted alkyl; R₄ is —H, —NR₁₃R₁₄ or

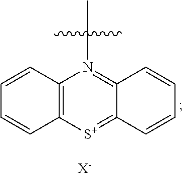

R₅ is —H or halo, —OR₁₅, alkyl or substituted alkyl; R₆ is —H, —NR₁₆R₁₇, —OR₁₈, alkyl, substituted alkyl, alkenyl, substituted alkenyl, alkynyl, substituted alkynyl, heteroalkyl, substituted heteroalkyl or halo; R₇ and R₈ are independently alkyl, substituted alkyl, alkenyl, substituted alkenyl, alkynyl, substituted alkynyl, heteroalkyl, substituted heteroalkyl or taken together with the atom to which they are bonded form a cycloheteroalkyl, substituted cycloheteroalkyl, cycloheteroalkenyl, substituted cycloheteroalkenyl, bicycloheteroalkyl, substituted bicycloheteroalkyl, spirocycloheteroalkyl or a substituted spirocycloheteroalkyl ring; R₁₀ and R₁₁ are independently alkyl, substituted alkyl, alkenyl, substituted alkenyl, alkynyl, substituted alkynyl, heteroalkyl, substituted heteroalkyl or taken together with the atom to which they are bonded form a cycloheteroalkyl, substituted cycloheteroalkyl, cycloheteroalkenyl, substituted cycloheteroalkenyl, bicycloheteroalkyl, substituted bicycloheteroalkyl, spirocycloheteroalkyl or a substituted spirocycloheteroalkyl ring; R₁₆ and R₁₇ are independently alkyl, substituted alkyl, alkenyl, substituted alkenyl, alkynyl, substituted alkynyl, heteroalkyl, substituted heteroalkyl or taken together with the atom to which they are bonded form a cycloheteroalkyl, substituted cycloheteroalkyl, cycloheteroalkenyl, substituted cycloheteroalkenyl, bicycloheteroalkyl, substituted bicycloheteroalkyl, spirocycloheteroalkyl or a substituted spirocycloheteroalkyl ring; R₉, R₁₂, R₁₅ and R₁₈ are independently alkyl, substituted alkyl, alkenyl, substituted alkenyl, alkynyl, substituted alkynyl, aryl, substituted aryl, heteroaryl, substituted aryl, heteroalkyl or substituted heteroalkyl; and X— is a pharmaceutically acceptable salt.

In some aspects, the following compounds are excluded: provided that when R₂ and R₄ both form a C₅-C₇ cycloheteroalkyl or C₅-C₇ substituted cycloheteroalkyl ring that R₁ is not H when R₆ is t-butyl, R₁ is not H or methyl when R₆ is H except when R₅ is —CH₂Boc or —OMe, R₁ is not methyl or ethyl when R₆ is ethyl; provided that none of R₁₀, R₁₁, R₁₃ or R₁₄ are alkyl or substituted alkyl when R₁ and R₆ are independently —H, alkyl, alkenyl or haloalkyl if either R₁₀ and R₁₁ or R₁₃ and R₁₄ form a C₅-C₇ cycloheteroalkyl or C₅-C₇ substituted cycloheteroalkyl ring; provided that at least one of R₁ and R₆ is not —H when one of R₂ and R₄ is —H; and provided that when R₂ is —NR₁₀R₁₁ and R₄ is —NR₁₃R₁₄ and any of R₁₀, R₁₁, R₁₃ or R₁₄ are substituted alkyl that none of R₁₀, R₁₁, R₁₃ or R₁₄ are substituted with —OR₁₉, —SR₂₀ or —NR₂₁ where R₁₉, R₂₀ or R₂₁ are independently alkyl, alkenyl or aryl. It should be understood that the above provisos may operate together or independently or not at all.

In some aspects, R₂ is —NR₉R₁₀ and R₄ is —NR₁₁R₁₂. In other aspects, R₉ and R₁₀ taken together with the atom to which they are bonded form a bicycloheteroalkyl or substituted bicycloheteroalkyl ring and R₁₁ and R₁₂ taken together with the atom to which they are bonded form a bicycloheteroalkyl or substituted bicycloheteroalkyl ring.

In some aspects, R₁, R₃, R₅ and R₆ are —H. In other aspects, compounds having the structure:

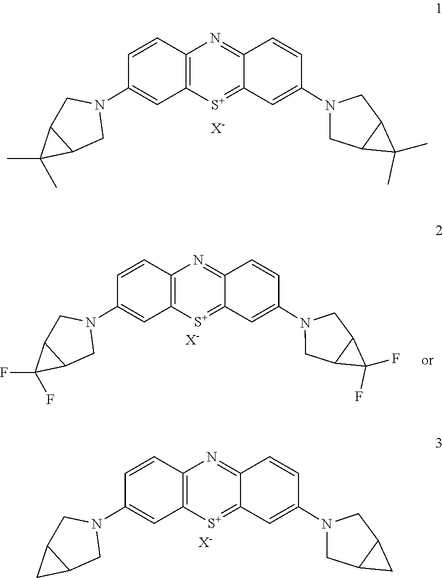

are provided. In still other aspects, the disclosed compound has structure 1. In still other aspects, the disclosed compound has structure 2. In still other aspects, the disclosed compound has structure 3.

In some aspects, R₃, R₅ and R₆ are —H. In other aspects, compounds having the structure:

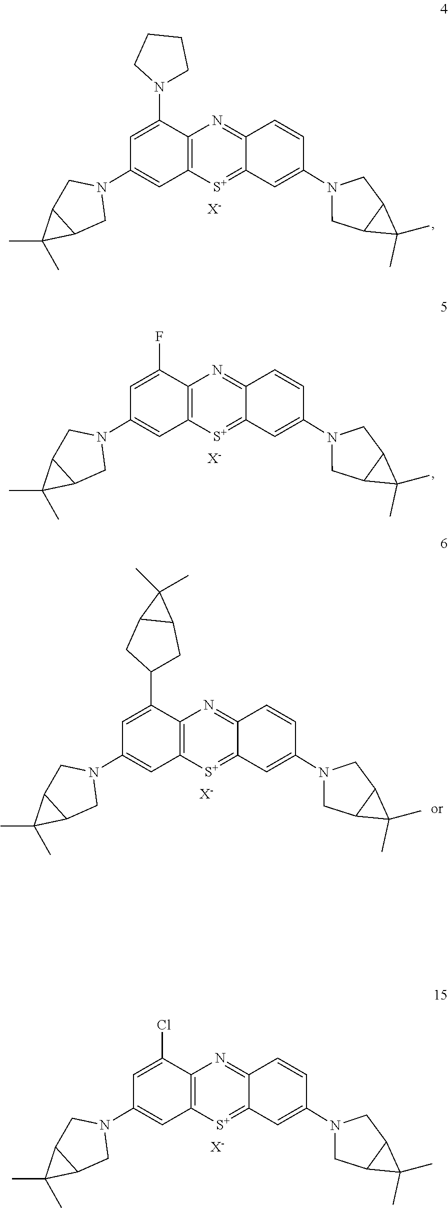

are provided. In still other aspects, the disclosed compound has structure 4. In still other aspects, the disclosed compound has structure 5. In still other aspects, the disclosed compound has structure 6. In still other aspects, the disclosed compound has structure 15.

In some aspects, R₁, R₃ and R₆ are —H. In other aspects, compounds having the structure:

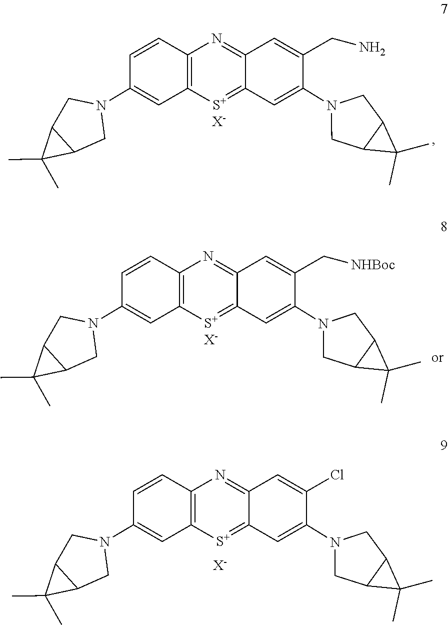

are provided. In still other aspects, the disclosed compound has structure 7. In still other aspects, the disclosed compound has structure 8. In some aspects, the disclosed compound has structure 9. In still other aspects, R₃ and R₅ are —H.

In some aspects, compounds having the structure:

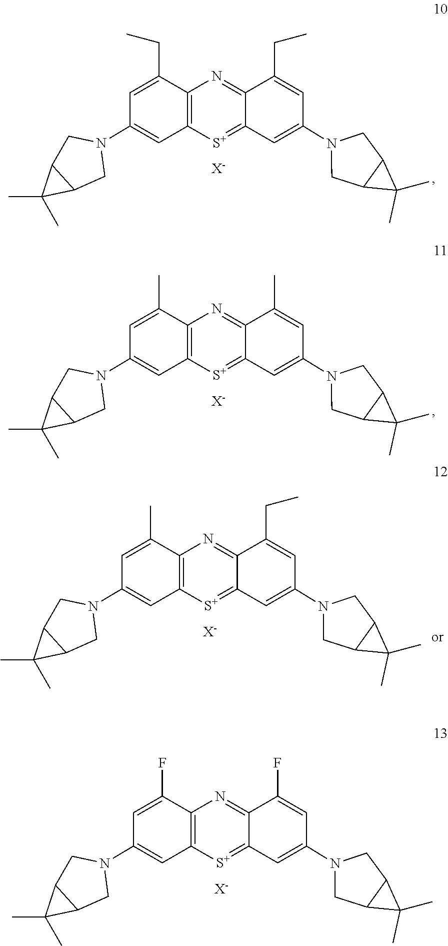

are provided. In other aspects, the disclosed compound has structure 10. In still other aspects, the disclosed compound has structure 11. In still other aspects, the disclosed compound has structure 12. In still other aspects, the disclosed compound has structure 13.

In some aspects, R₅ is —H. In other aspects, a compound having the structure:

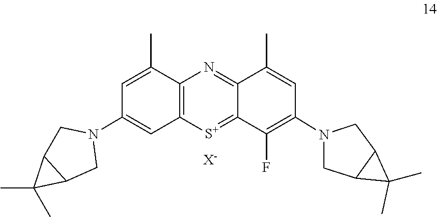

is provided.

In some aspects, R₉ and R₁₀ taken together with the atom to which they are bonded form a spirocycloheteroalkyl or substituted spirocycloheteroalkyl ring and R₁₁ and R₁₂ taken together with the atom to which they are bonded form a spirocycloheteroalkyl or substituted spirocycloheteroalkyl ring. In other aspects, a compound having the structure:

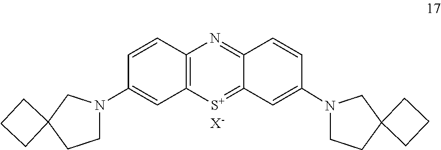

is provided.

In some aspects, R₂ is

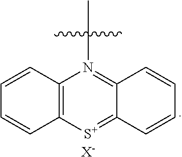

In other aspects, compounds having the structure:

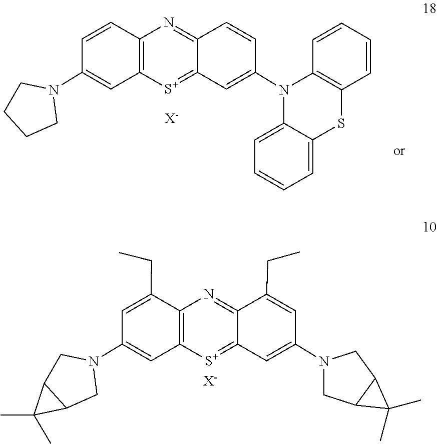

are provided. In still other aspects, the disclosed compound has structure 18. In other aspects, the disclosed compound has structure 19.

In some aspects, R₉ and R₁₀ taken together with the atom to which they are bonded form a cyclo heterocycloalkyl, substituted cycloheteroalkyl, cycloheteroalkenyl or substituted cycloheteroalkenyl ring and R₁₁ and R₁₂ taken together with the atom to which they are bonded form a cycloheteroalkyl, substituted cycloheteroalkyl, cycloheteroalkenyl or substituted cycloheteroalkenyl ring. In other aspects, compounds having the structure:

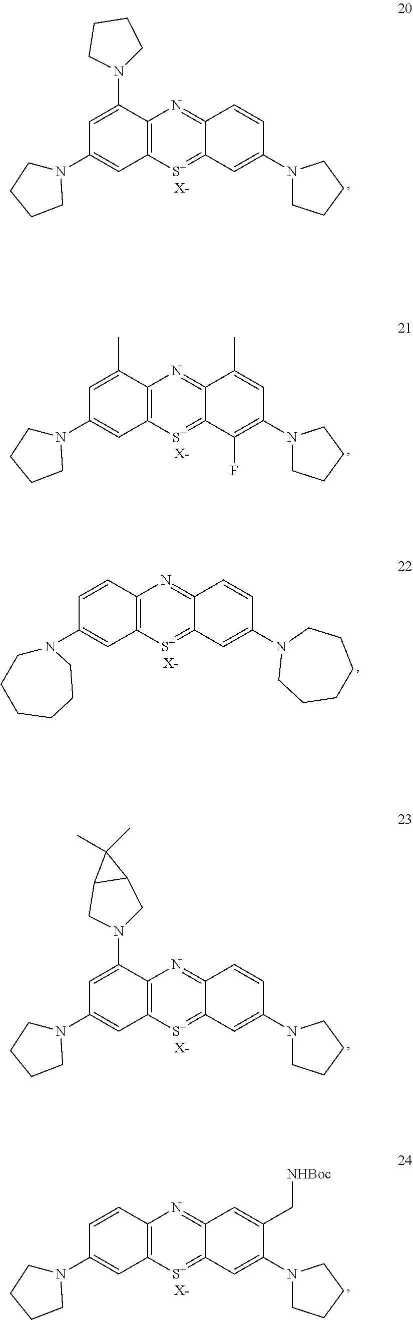

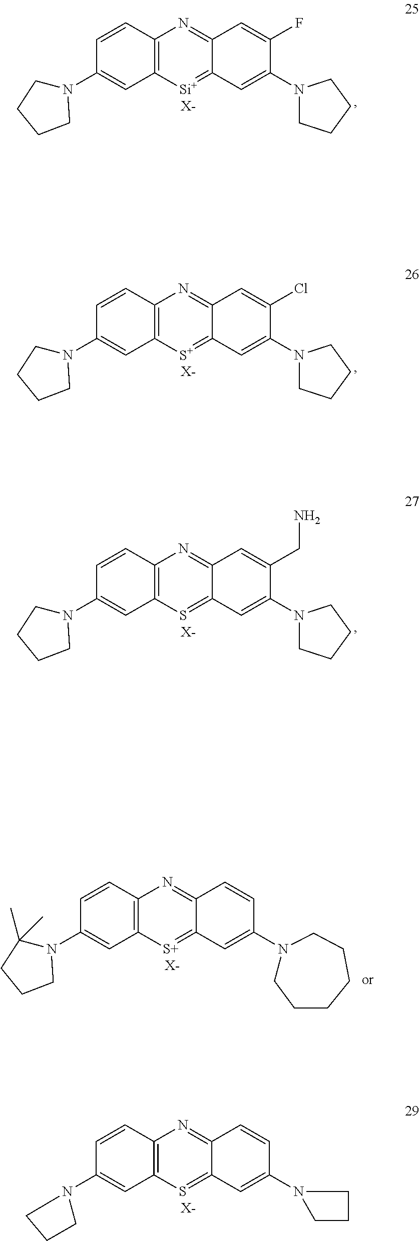

are provided. In still other aspects, the disclosed compound has structure 20. In still other aspects, the disclosed compound has structure 21. In still other aspects, the disclosed compound has structure 22. In still other aspects, the disclosed compound has structure 23. In still other aspects, the disclosed compound has structure 24. In still other aspects, the disclosed compound has structure 25. In still other aspects, the disclosed compound has structure 27. In still other aspects, the disclosed compound has structure 28. In still other aspects, the disclosed compound has structure 29.

In some aspects, R₉ and R₁₀ taken together with the atom to which they are bonded form a bicycloheteroalkyl or substituted bicycloheteroalkyl ring and R₁₁ and R₁₂ are alkyl or taken together with the atom to which they are bonded form a cycloheteroalkyl, substituted cycloheteroalkyl, cycloheteroalkenyl or substituted cycloheteroalkenyl ring. In other aspects, compounds having the structure:

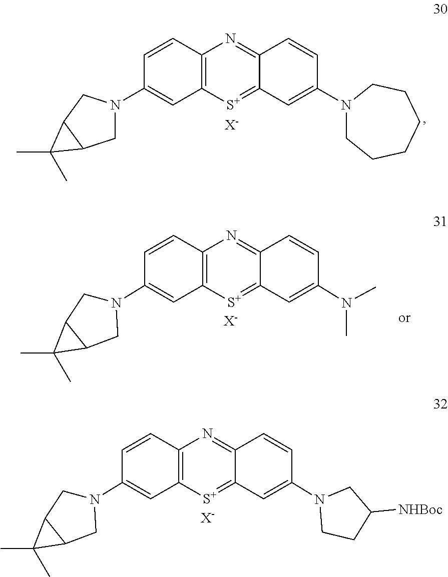

are provided. In still other aspects, the disclosed compound has structure 30. In still other aspects, the disclosed compound has structure 31. In still other aspects, the disclosed compound has structure 32.

In some aspects, R₉ and R₁₀ are alkyl and R₁₁ and R₁₂ taken together with the atom to which they are bonded form a cycloheteroalkyl, substituted cycloheteroalkyl, cycloheteroalkenyl or substituted cycloheteroalkenyl ring. In other aspects, compounds having the structure:

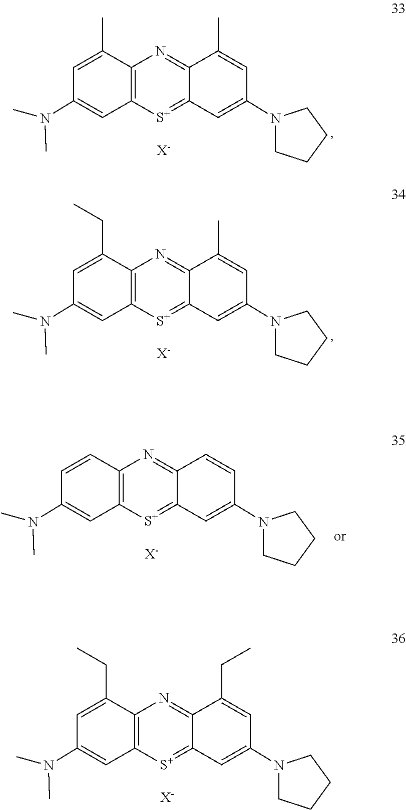

are provided. In still other aspects, the disclosed compound has structure 33. In still other aspects, the disclosed compound has structure 34. In still other aspects, the disclosed compound has structure 35. In still other aspects, the disclosed compound has structure 36.

In some aspects, R₉ and R₁₀ taken together with the atom to which they are bonded form a cycloheteroalkyl, substituted cycloheteroalkyl, cycloheteroalkenyl or substituted cycloheteroalkenyl ring and R₁₁ and R₁₂ are —H. In other aspects, compounds having the structure:

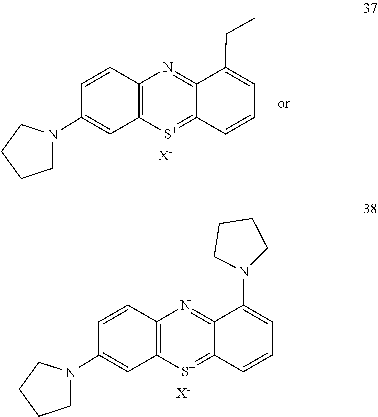

are provided. In still other aspects, the disclosed compound has structure 37. In still other aspects, the disclosed compound has structure 38.

In some aspects, R₁₁ and R₁₂ together with the atom to which they are attached form a saturated substituted pyrrolidinyl-1-yl ring and R₁₃ and R₁₄ together with the atom to which they are attached form a saturated substituted pyrrolidinyl-1-yl ring. In other aspects, compounds having the structure:

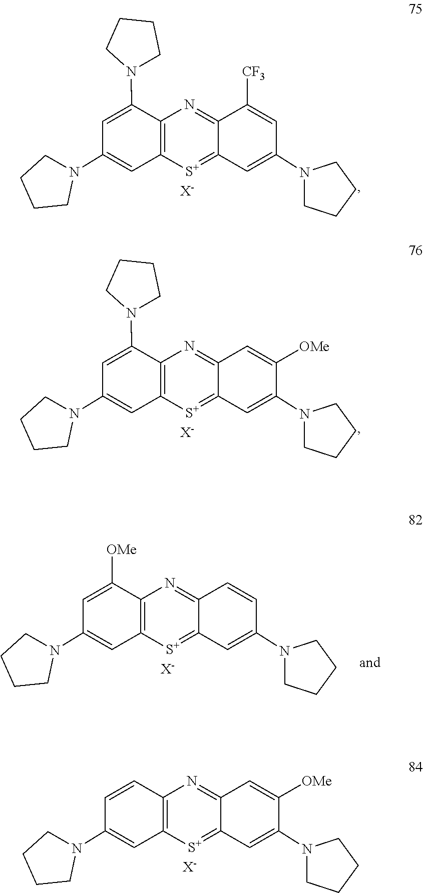

are provided. In still other aspects, the disclosed compound has structure 75. In still other aspects, the disclosed compound has structure 76. In still other aspects, the disclosed compound has structure 82. In still other aspects, the disclosed compound has structure 84.

In some aspects, a compound of Formula (II):

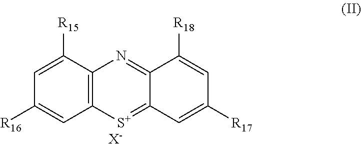

or pharmaceutically acceptable salts, solvates or hydrates thereof where R₁₅ is H, alkyl or halo; R₁₆ is —NR₁₉R₂₀; R₁₇ is —NR₂₁R₂₂; R₁₈ is —H, alkyl or halo; R₁₉ and R₂₀ are alkyl, or together with the atom to which they are attached form a saturated pyrrolidinyl-1-yl, saturated substituted pyrrolidinyl-1-yl, unsaturated pyrrolidinyl-1-yl, unsaturated substituted pyrrolidinyl-1-yl, saturated piperazin-1-yl, saturated substituted piperazin-1-yl, unsaturated piperazin-1-yl ring or unsaturated substituted piperazin-1-yl ring; R₂₁ and R₂₂ are alkyl, or together with the atom to which they are attached form a saturated pyrrolidinyl-1-yl, saturated substituted pyrrolidinyl-1-yl, unsaturated pyrrolidinyl-1-yl, unsaturated substituted pyrrolidinyl-1-yl, saturated piperazin-1-yl, saturated substituted piperazin-1-yl, unsaturated piperazin-1-yl ring or unsaturated substituted piperazin-1-yl ring; and X— is a pharmaceutically acceptable salt; provided that the compound of Formula (II) is not

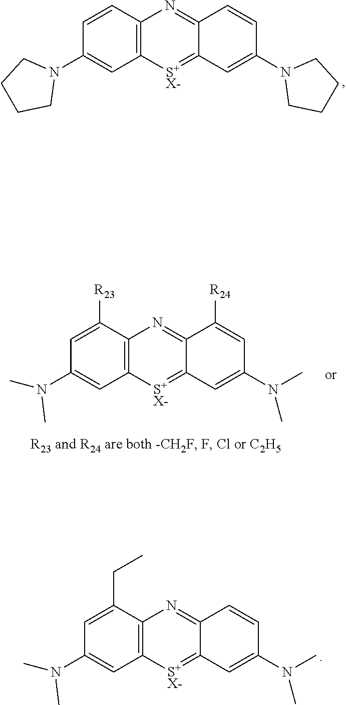

In some aspects, R₁₉ and R₂₀ together with the atom to which they are attached form a saturated pyrrolidinyl-1-yl ring and R₂₁ and R₂₂ together with the atom to which they are attached form a saturated pyrrolidinyl-1-yl ring. In other aspects, compounds having the structure:

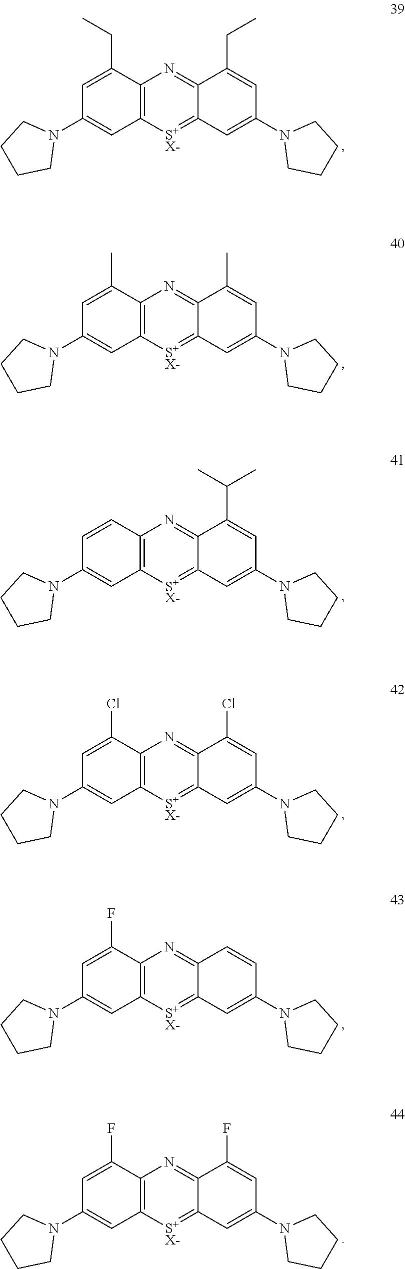

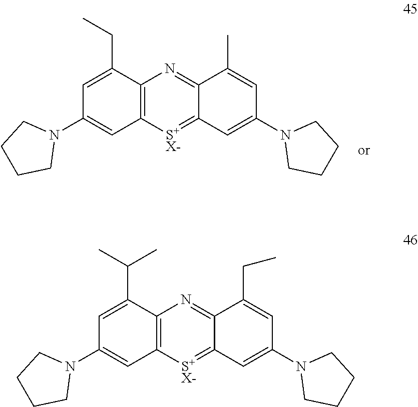

are provided. In still other aspects, the disclosed compound has structure 39. In still other aspects, the disclosed compound has structure 40. In still other aspects, the disclosed compound has structure 41. In still other aspects, the disclosed compound has structure 42. In still other aspects, the disclosed compound has structure 43. In still other aspects, the disclosed compound has structure 44. In still other aspects, the disclosed compound has structure 45. In still other aspects, the disclosed compound has structure 46.

In some aspects, R₁₉ and R₂₀ together with the atom to which they are attached form a saturated substituted pyrrolidinyl-1-yl ring and R₂₁ and R₂₂ together with the atom to which they are attached form a saturated substituted pyrrolidinyl-1-yl ring. In other aspects, compounds having the structure:

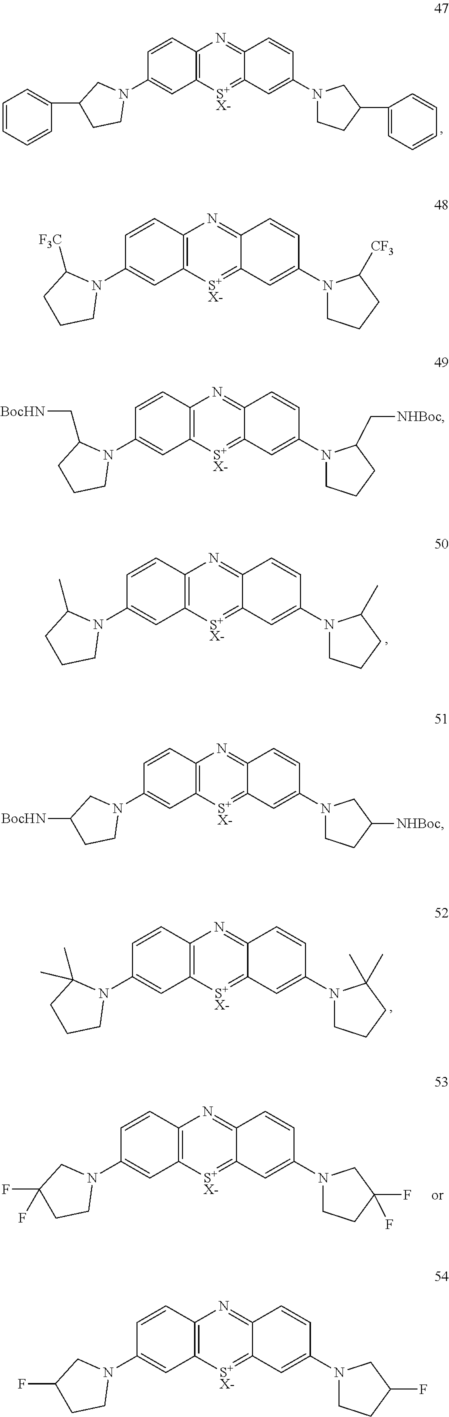

are provided. In still other aspects, the disclosed compound has structure 47. In still other aspects, the disclosed compound has structure 48. In still other aspects, the disclosed compound has structure 49. In still other aspects, the disclosed compound has structure 50. In still other aspects, the disclosed compound has structure 51. In still other aspects, the disclosed compound has structure 52. In still other aspects, the disclosed compound has structure 53. In still other aspects, the disclosed compound has structure 54.

In some aspects, R₁₉ and R₂₀ together with the atom to which they are attached form a saturated pyrrolidinyl-1-yl ring and R₂₁ and R₂₂ together with the atom to which they are attached form a saturated substituted pyrrolidinyl-1-yl ring. In other aspects, compounds having the structure:

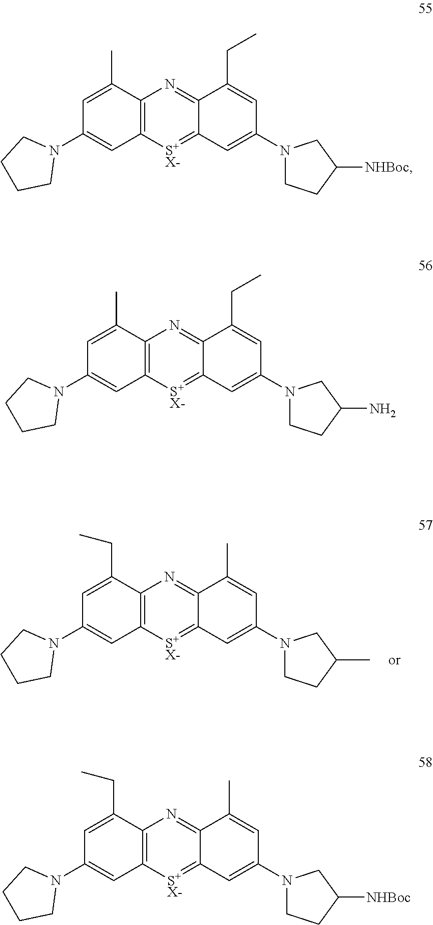

are provided. In still other aspects, the disclosed compound has structure 55. In still other aspects, the disclosed compound has structure 56. In still other aspects, the disclosed compound has structure 57. In still other aspects, the disclosed compound has structure 58.

In some aspects, R₁₉ and R₂₀ are alkyl and R₂₁ and R₂₂ are alkyl. In other aspects, compound having the structure:

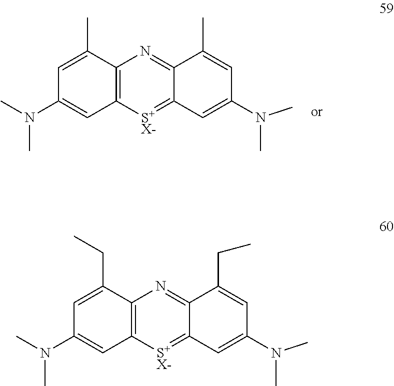

are provided. In still other aspects, the disclosed compound has structure 59. In still other aspects, the disclosed compound has structure 60.

In some aspects, R₁₉ and R₂₀ together with the atom to which they are attached form an unsaturated substituted pyrrolidinyl-1-yl ring and R₂₁ and R₂₂ together with the atom to which they are attached form an unsaturated substituted pyrrolidinyl-1-yl ring. In other aspects, a compound having the structure:

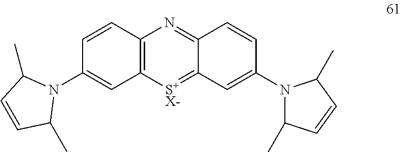

is provided.

In some aspects, R₁₉ and R₂₀ together with the atom to which they are attached form a saturated piperazin-1-yl and R₂₁ and R₂₂ or together with the atom to which they are attached form a saturated piperazin-1-yl ring. In other aspects, a compound having the structure:

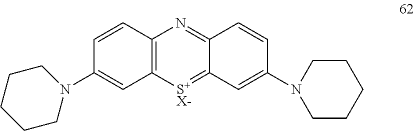

is provided.

TABLE 1

| | |
|---|---|
| 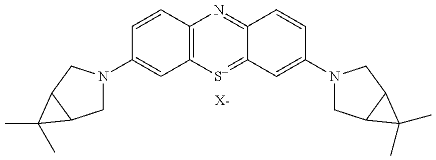 | 1 |
| 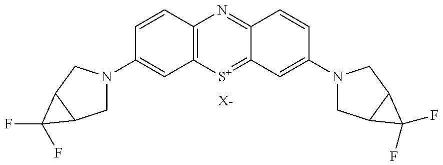 | 2 |
| 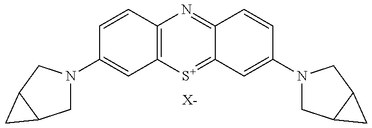 | 3 |
| 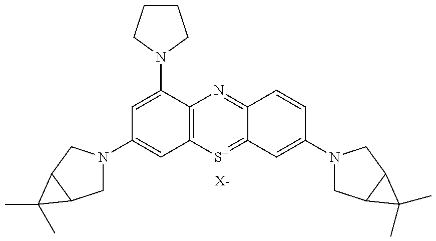 | 4 |
| 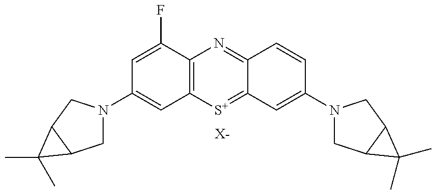 | 5 |
| 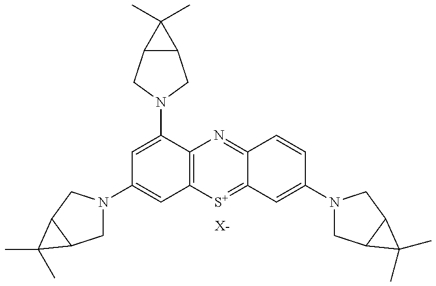 | 6 |
| 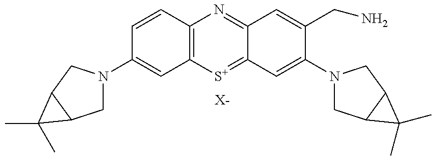 | 7 |
| 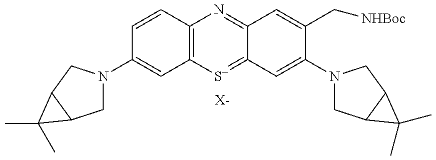 | 8 |
| 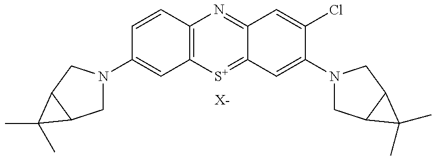 | 9 |
| 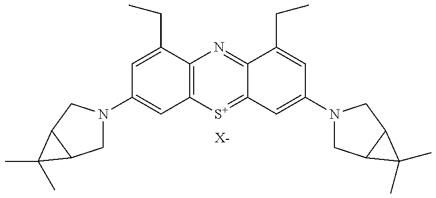 | 10 |
| 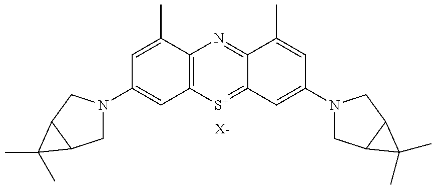 | 11 |
| 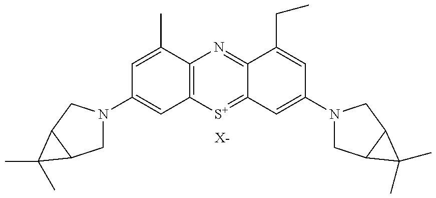 | 12 |
| 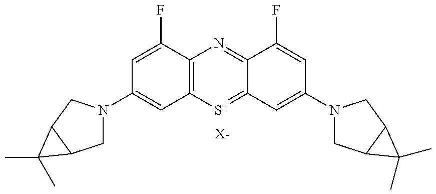 | 13 |
| 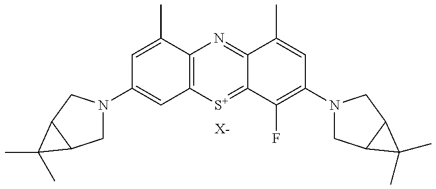 | 14 |
| 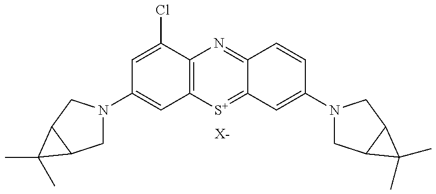 | 15 |
| 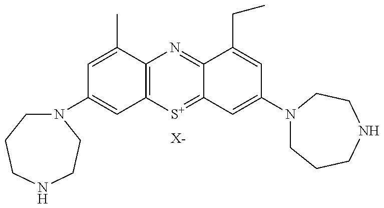 | 16 |
| 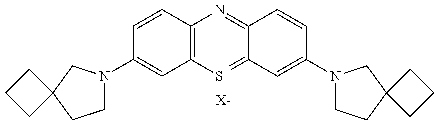 | 17 |
| 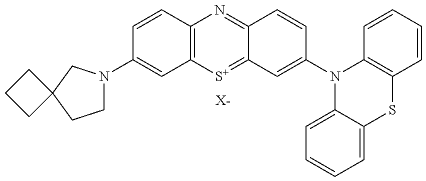 | 18 |
| 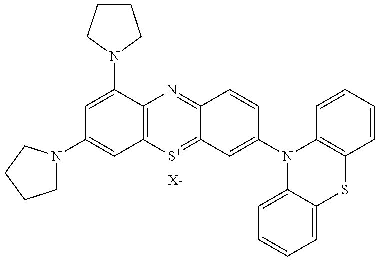 | 19 |
| 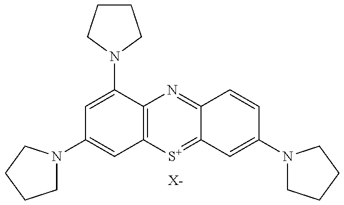 | 20 |
| 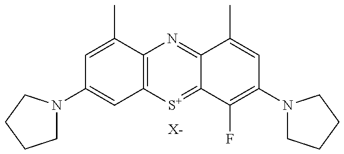 | 21 |
| 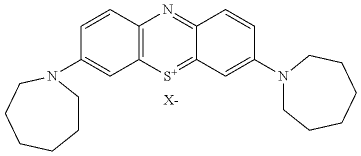 | 22 |
| 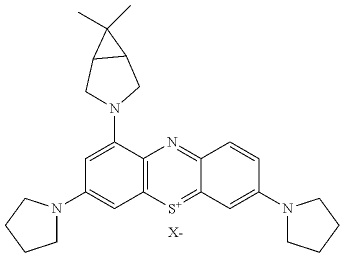 | 23 |
| 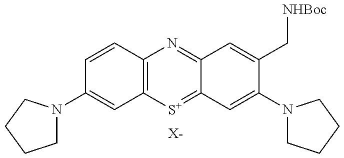 | 24 |
| 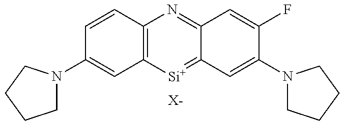 | 25 |
| 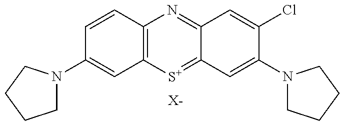 | 26 |
| 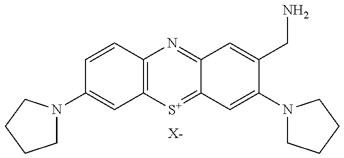 | 27 |
| 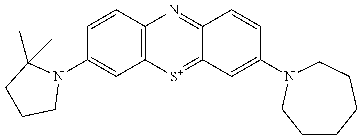 [SMILES: CC1(C)CCCN1c1ccc2nc3ccc(N4CCCCCC4)cc3[s+]c2c1] | 28 |
| 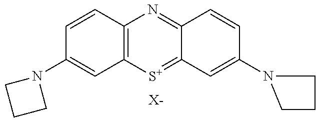 | 29 |
| 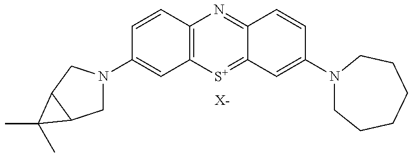 | 30 |
| 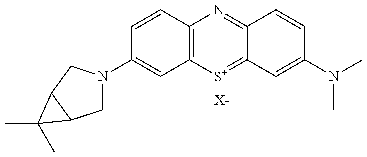 | 31 |
| 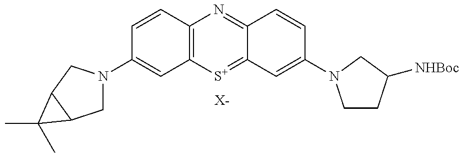 | 32 |
| 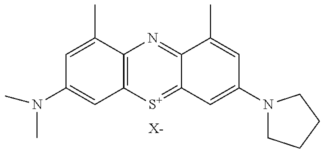 | 33 |
| 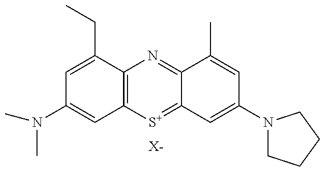 | 34 |
| 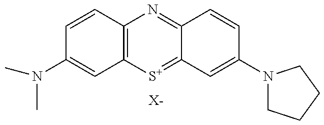 | 35 |
| 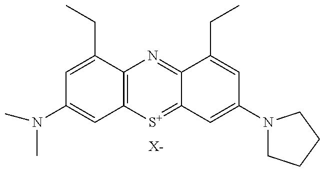 | 36 |
| 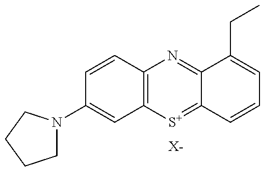 | 37 |
| 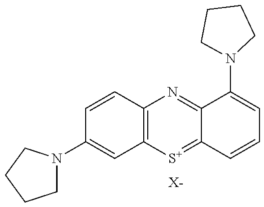 | 38 |
| 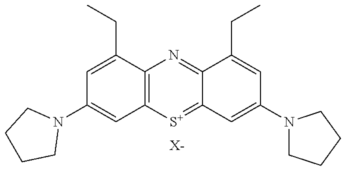 | 39 |
| 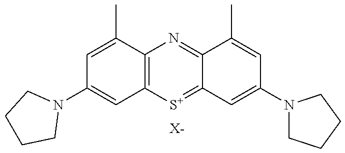 | 40 |
| 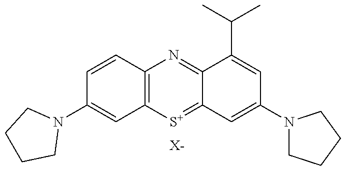 | 41 |
| 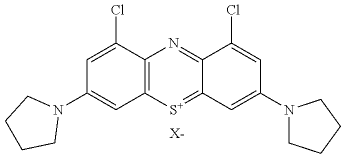 | 42 |
| 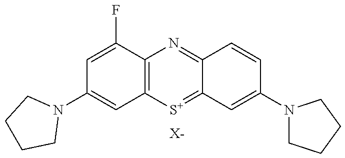 | 43 |
| 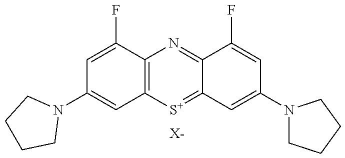 | 44 |
| 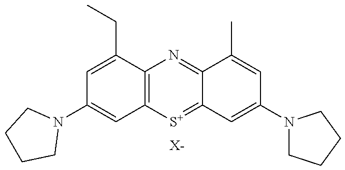 | 45 |
| 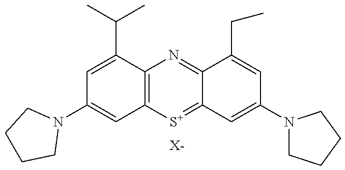 | 46 |
| 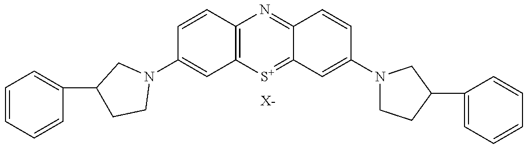 | 47 |
| 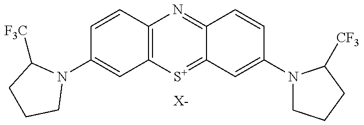 | 48 |
| 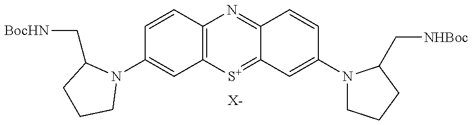 | 49 |
| 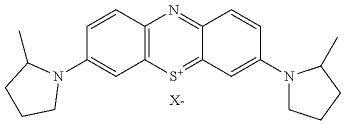 | 50 |
| 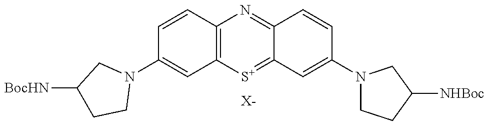 | 51 |
| 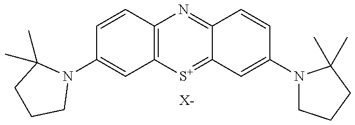 | 52 |
| 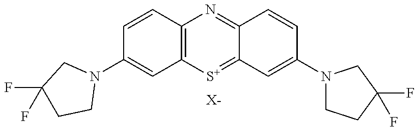 | 53 |
| 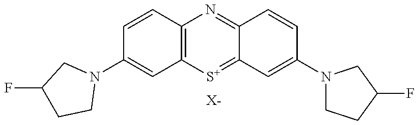 | 54 |
| 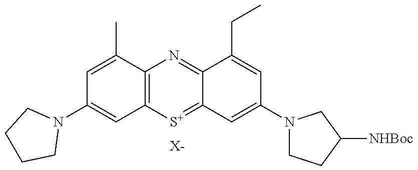 | 55 |
| 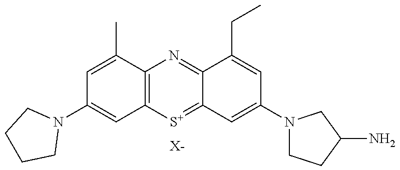 | 56 |
| 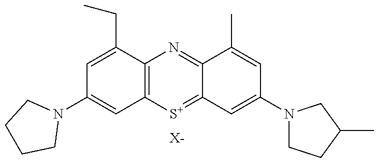 | 57 |
| 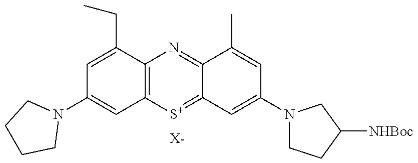 | 58 |
| 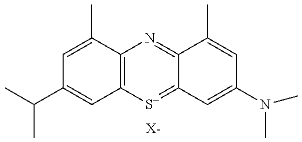 | 59 |
| 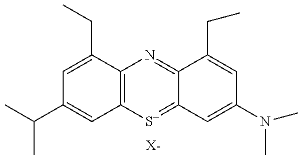 | 60 |
| 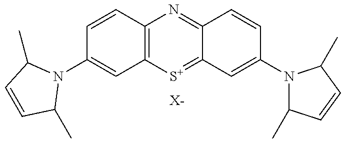 | 61 |
| 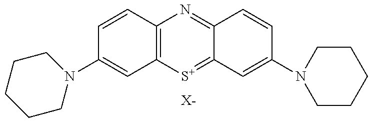 | 62 |
| 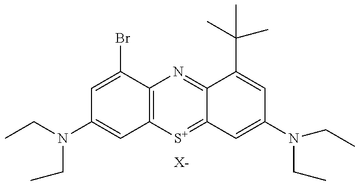 | 63 |
| 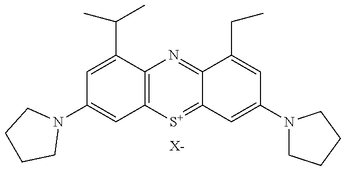 | 64 |
| 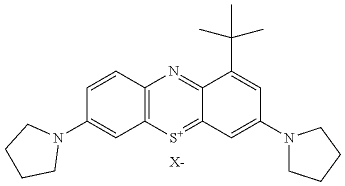 | 65 |
| 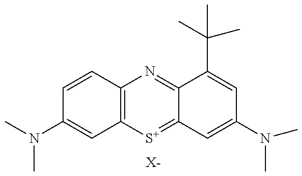 | 66 |
| 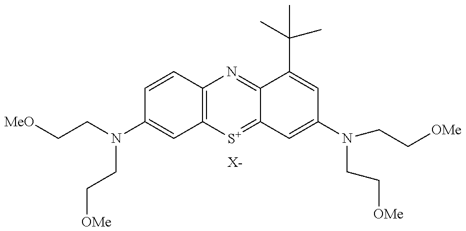 | 67 |
| 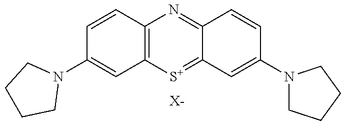 | 68 |
| 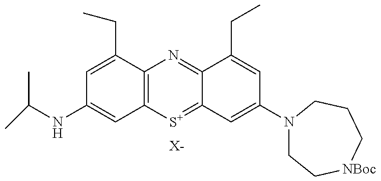 | 69 |
| 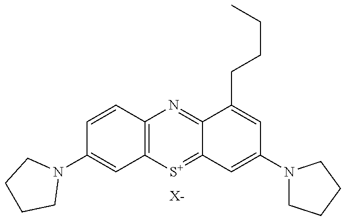 | 70 |
| 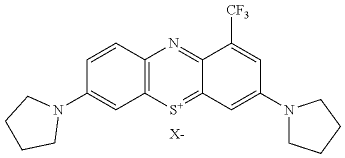 | 71 |
| 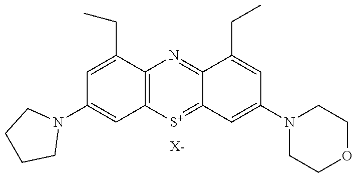 | 72 |
| 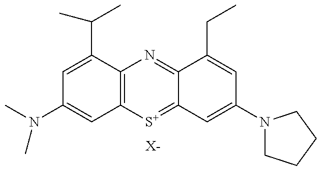 | 73 |
| 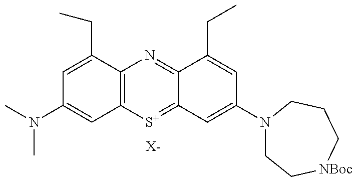 | 74 |
| 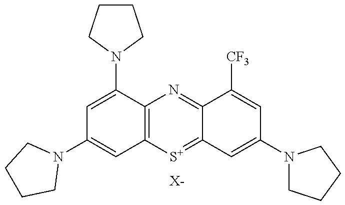 | 75 |
| 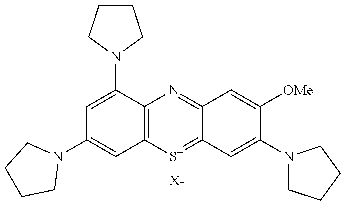 | 76 |
| 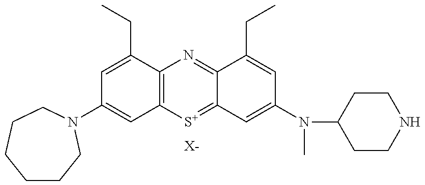 | 77 |
| 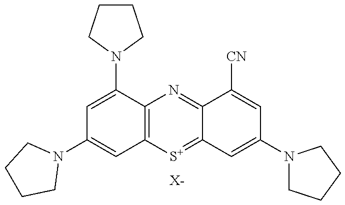 | 78 |
| 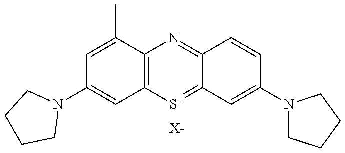 | 79 |
| 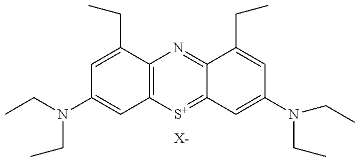 | 80 |
| 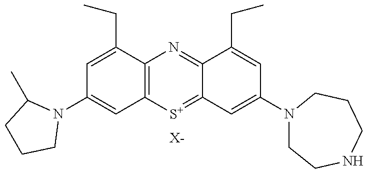 | 81 |
| 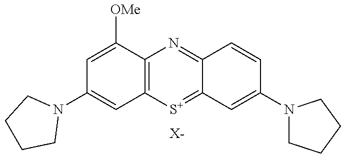 | 82 |
| 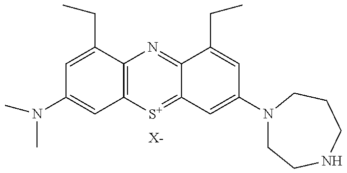 | 83 |
| 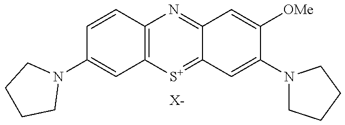 | 84 |
| 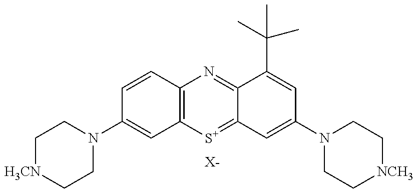 | 85 |
| 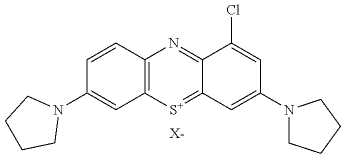 | 86 |
| 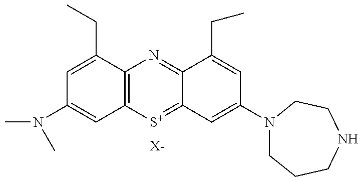 | 87 |
| 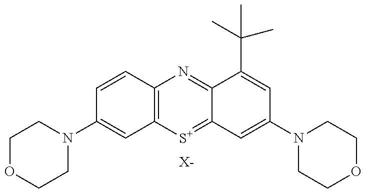 | 88 |
| 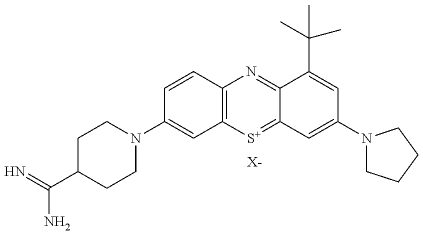 | 89 |
| 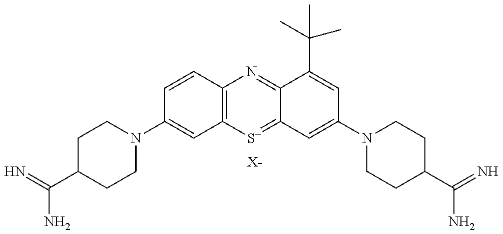 | 90 |
| 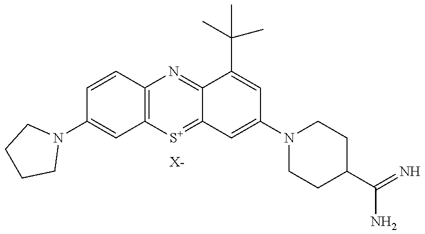 | 91 |
| 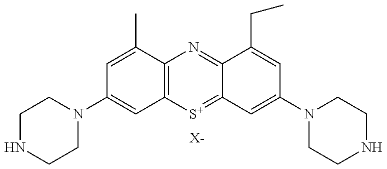 | 92 |
| 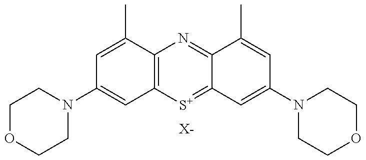 | 93 |
| 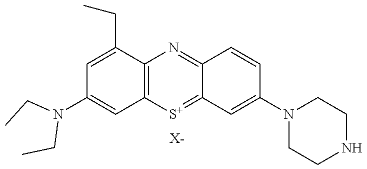 | 94 |
| 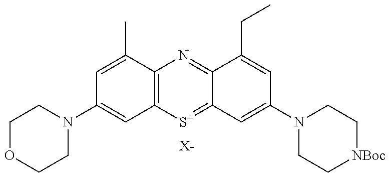 | 95 |
| 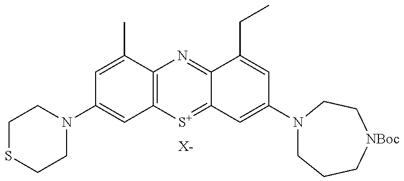 | 96 |
| 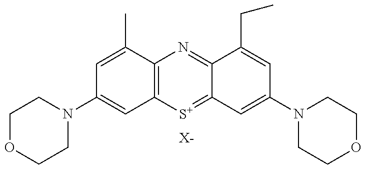 | 97 |
| 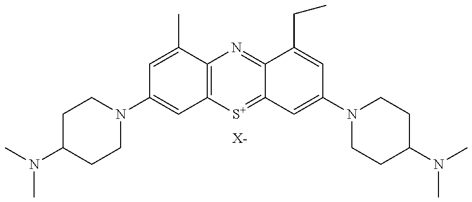 | 98 |
| 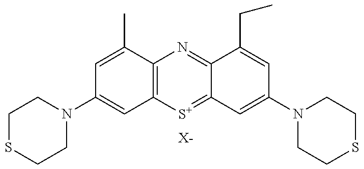 | 99 |
| 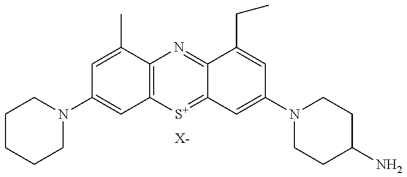 | 100 |
| 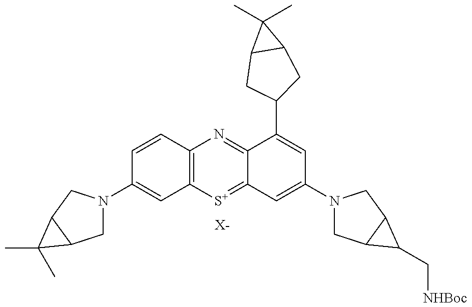 | 101 |
| 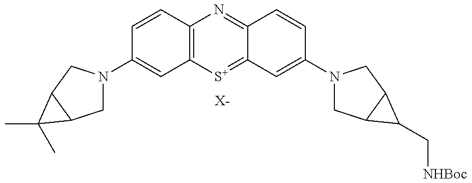 | 102 |
| 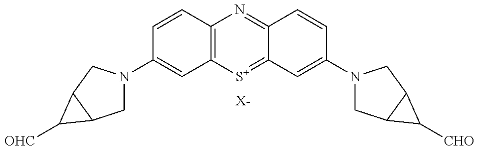 | 103 |
| 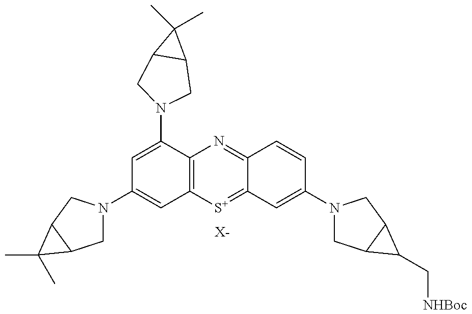 | 104 |
| 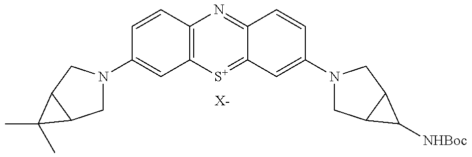 | 105 |
| 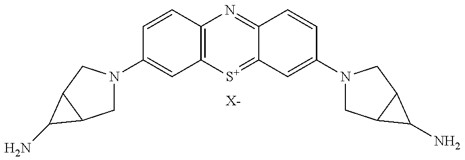 | 106 |
| 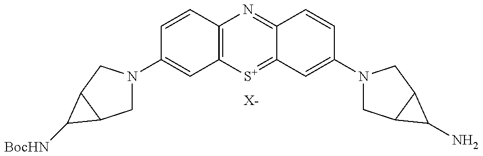 | 107 |
| 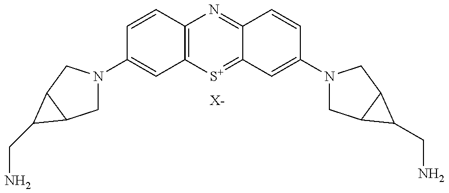 | 108 |
| 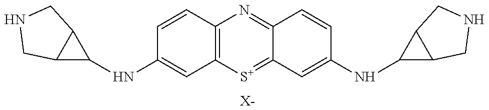 | 109 |
| 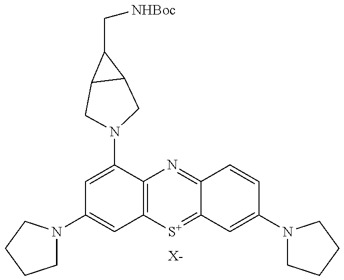 | 110 |
| 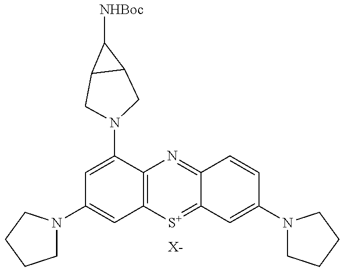 | 111 |
| 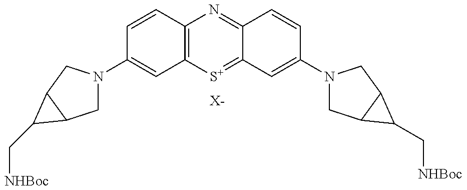 | 112 |
| 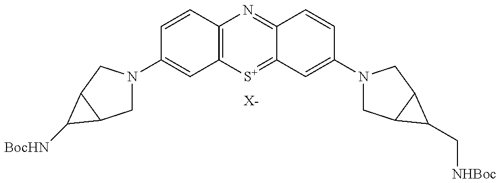 | 113 |
| 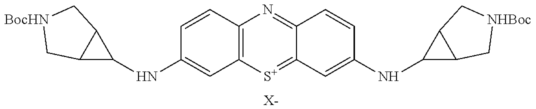 | 114 |
| 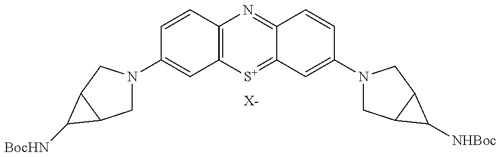 | 115 |
| 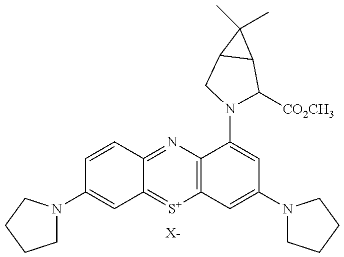 | 116 |
| 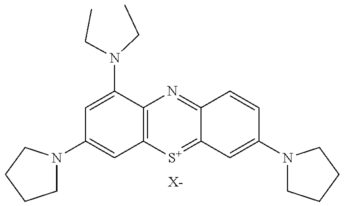 | 117 |
| 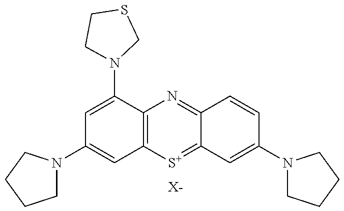 | 118 |
| 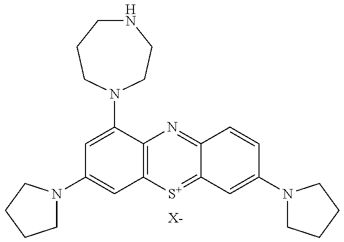 | 119 |
| 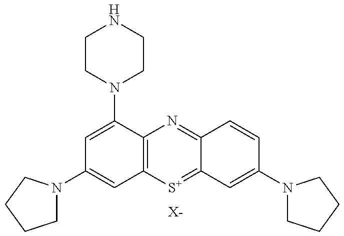 | 120 |
| 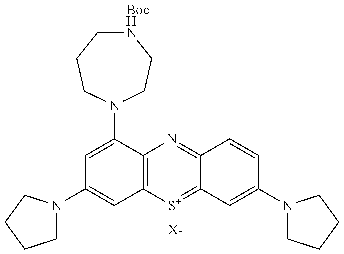 | 121 |
| 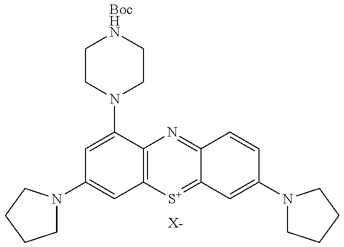 | 122 |
| 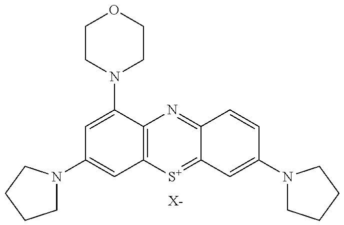 | 123 |
| 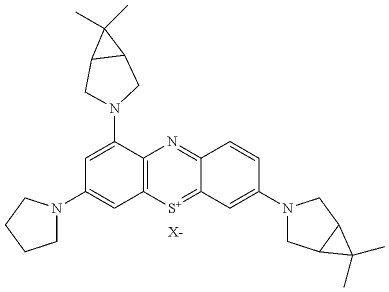 | 124 |
| 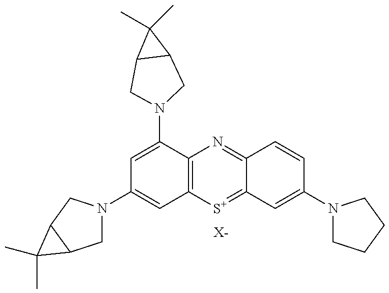 | 125 |
| 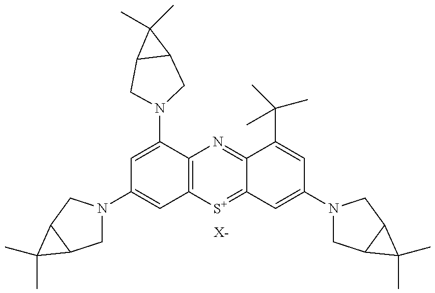 | 126 |
| 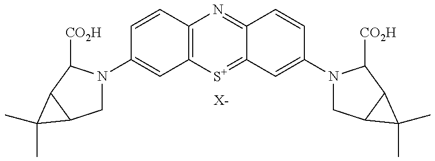 | 127 |
| 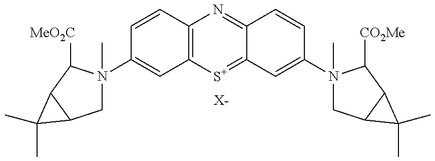 | 128 |
| 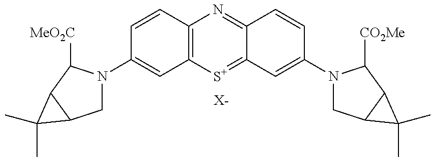 | 129 |
| 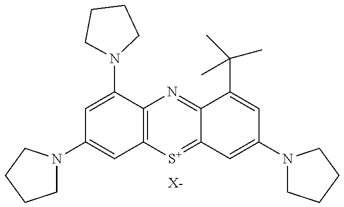 | 130 |
| 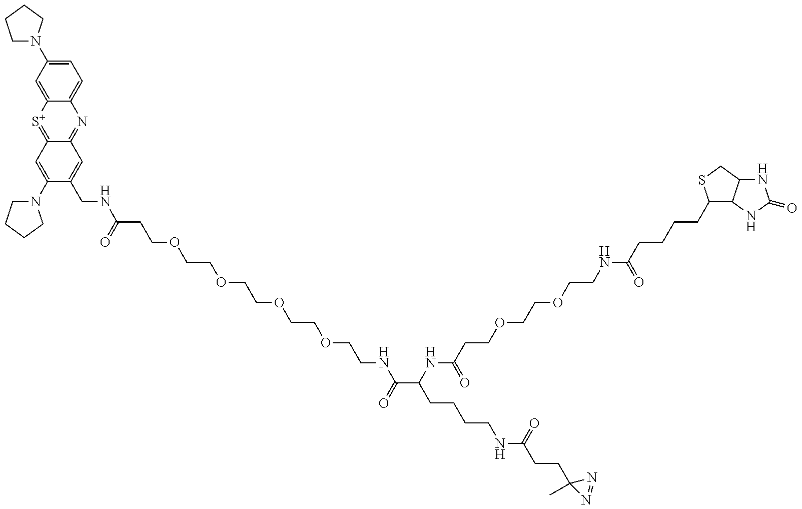 [SMILES: CC1(CCC(=O)NCCCCC(NC(=O)CCOCCOCCNC(=O)CCCCC2SCC3NC(=O)NC32)C(=O)NCCOCCOCCOCCOCCC(=O)NCc2cc3nc4ccc(N5CCCC5)cc4[s+]c3cc2N2CCCC2)N=N1] | 131 |
| 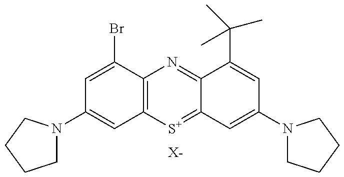 | 132 |
| 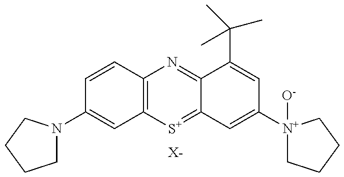 | 133 |
| 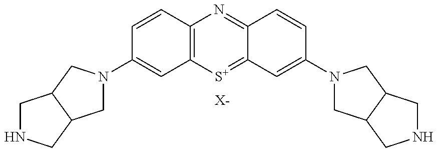 | 134 |
| 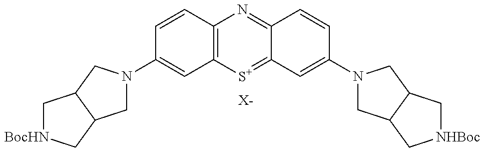 | 135 |
| 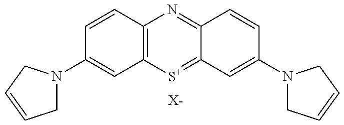 | 136 |
| 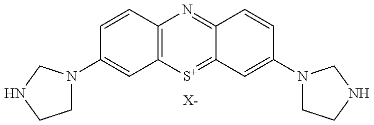 | 137 |
| 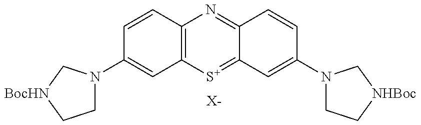 | 138 |
| 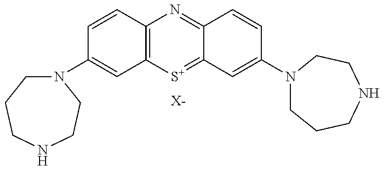 | 139 |
| 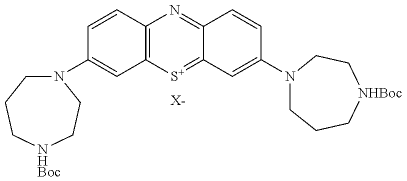 | 140 |
| 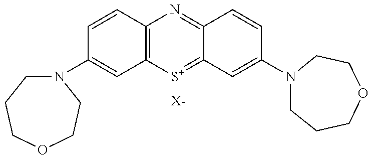 | 141 |
| 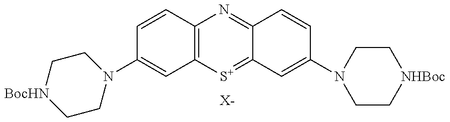 | 142 |
| 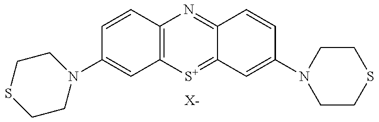 | 143 |
| 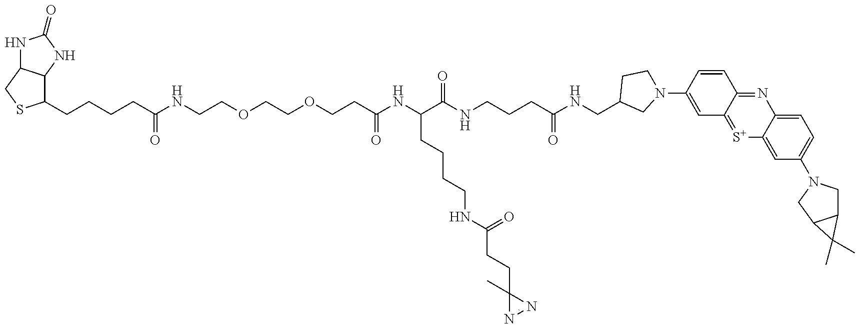 [SMILES: CC1(CCC(=O)NCCCCC(NC(=O)CCOCCOCCNC(=O)CCCCC2SCC3NC(=O)NC32)C(=O)NCCCC(=O)NCC2CCN(c3ccc4nc5ccc(N6CC7C(C6)C7(C)C)cc5[s+]c4c3)C2)N=N1] | 144 |
| 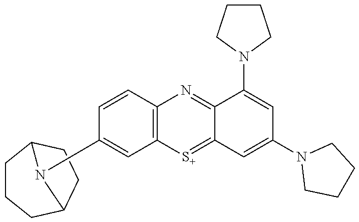 [SMILES: c1cc2nc3c(N4CCCC4)cc(N4CCCC4)cc3[s+]c2cc1N1C2CCCC1CC2] | 145 |
| 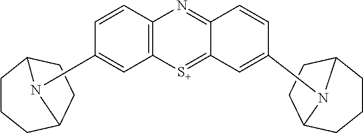 [SMILES: c1cc2nc3ccc(N4C5CCCC4CC5)cc3[s+]c2cc1N1C2CCCC1CC2] | 146 |
| 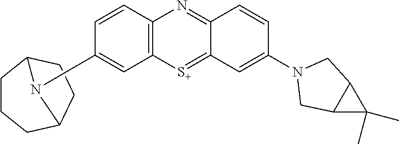 [SMILES: CC1(C)C2CN(c3ccc4nc5ccc(N6C7CCCC6CC7)cc5[s+]c4c3)CC21] | 147 |
| 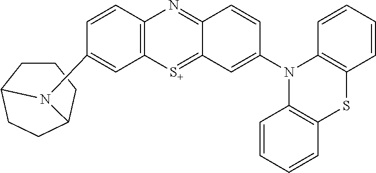 [SMILES: c1ccc2c(c1)Sc1ccccc1N2c1ccc2nc3ccc(N4C5CCCC4CC5)cc3[s+]c2c1] | 148 |
| 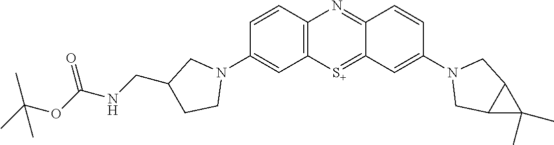 [SMILES: CC(C)(C)OC(=O)NCC1CCN(c2ccc3nc4ccc(N5CC6C(C5)C6(C)C)cc4[s+]c3c2)C1] | 149 |
| 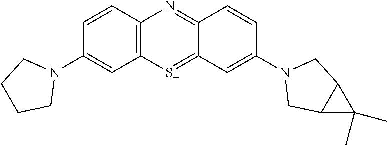 [SMILES: CC1(C)C2CN(c3ccc4nc5ccc(N6CCCC6)cc5[s+]c4c3)CC21] | 150 |
| 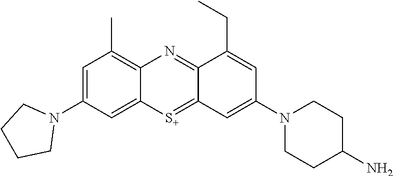 [SMILES: CCc1cc(N2CCC(N)CC2)cc2[s+]c3cc(N4CCCC4)cc(C)c3nc12] | 151 |
| 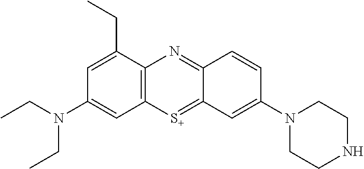 [SMILES: CCc1cc(N(CC)CC)cc2[s+]c3cc(N4CCNCC4)ccc3nc12] | 152 |

The compounds above can be made by well know procedures some of which are exemplified in the experimental section.

### Compositions and Methods of Administration

The compositions provided herein contain therapeutically effective amounts of one or more of the compounds provided herein that are useful in the prevention, treatment, or amelioration of one or more of the symptoms of diseases or disorders described herein and a vehicle. Vehicles suitable for administration of the compounds provided herein include any such carriers known to those skilled in the art to be suitable for the particular mode of administration. In addition, the compounds may be formulated as the sole active ingredient in the composition or may be combined with other active ingredients.

Any method of administration may be used to administer the disclosed compounds. Such methods are well known to those skilled in the art and include, but are not limited to, the following routes: oral administration, transdermal administration, administration by inhalation, nasal administration, topical administration, in utero administration, intrahepatic administration, intravaginal administration, ophthalmic administration, intraoral administration, optic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed compound, a disclosed therapeutic agent, a disclosed pharmaceutical composition, or a combination thereof can comprise administration directly into the CNS (e.g., intraparenchymal, intracerebroventriular, intrathecal cisternal, intrathecal (lumbar), deep gray matter delivery, convection-enhanced delivery to deep gray matter) or the PNS. Administration can be continuous or intermittent.

The compositions contain one or more compounds provided herein. The compounds are, in some aspects, formulated into suitable preparations such as solutions, suspensions, tablets, dispersible tablets, pills, capsules, powders, sustained release formulations or elixirs, for oral administration or in sterile solutions or suspensions for parenteral administration, as well as topical administration, transdermal administration and oral inhalation via nebulizers, pressurized metered dose inhalers and dry powder inhalers. In some aspects, the compounds described above are formulated into compositions using techniques and procedures well known in the art (see, e.g., Ansel, Introduction to Pharmaceutical Dosage Forms, Seventh Edition (1999)).

In the compositions, effective concentrations of one or more compounds or derivatives thereof is (are) mixed with a suitable vehicle. The compounds may be derivatized as the corresponding salts, esters, enol ethers or esters, acetals, ketals, orthoesters, hemiacetals, hemiketals, acids, bases, solvates, ion-pairs, hydrates or prodrugs prior to formulation, as described above. The concentrations of the compounds in the compositions are effective for delivery of an amount, upon administration that treats, leads to prevention, or amelioration of one or more of the symptoms of diseases or disorders described herein. In some aspects, the compositions are formulated for single dosage administration. To formulate a composition, the weight fraction of a compound is dissolved, suspended, dispersed or otherwise mixed in a selected vehicle at an effective concentration such that the treated condition is relieved, prevented, or one or more symptoms are ameliorated.

The active compound is included in the vehicle in an amount sufficient to exert a therapeutically useful effect in the absence of undesirable side effects on the patient treated. The therapeutically effective concentration may be predicted empirically by testing the compounds in in vitro and in vivo systems well known to those of skill in the art and then extrapolated therefrom for dosages for humans. Human doses are then typically fine-tuned in clinical trials and titrated to response.

The concentration of active compound in the composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders as described herein.

In instances in which the compounds exhibit insufficient solubility, methods for solubilizing compounds may be used such as use of liposomes, prodrugs, complexation/chelation, nanoparticles, or emulsions or tertiary templating. Such methods are known to those of skill in this art, and include, but are not limited to, using co-solvents, such as dimethylsulfoxide (DMSO), using surfactants or surface modifiers, such as TWEEN®, complexing agents such as cyclodextrin or dissolution by enhanced ionization (i.e., dissolving in aqueous sodium bicarbonate). Derivatives of the compounds, such as prodrugs of the compounds may also be used in formulating effective compositions.

Upon mixing or addition of the compound(s), the resulting mixture may be a solution, suspension, emulsion or the like. The form of the resulting mixture depends upon a number of factors, including the intended mode of administration and the solubility of the compound in the selected vehicle. The effective concentration is sufficient for ameliorating the symptoms of the disease, disorder or condition treated and may be empirically determined.

The compositions are provided for administration to humans and animals in indication appropriate dosage forms, such as dry powder inhalers (DPIs), pressurized metered dose inhalers (pMDIs), nebulizers, tablets, capsules, pills, sublingual tapes/bioerodible strips, tablets or capsules, powders, granules, lozenges, lotions, salves, suppositories, fast melts, transdermal patches or other transdermal application devices/preparations, sterile parenteral solutions or suspensions, and oral solutions or suspensions, and oil-water emulsions containing suitable quantities of the compounds or derivatives thereof. The therapeutically active compounds and derivatives thereof are, in some aspects, formulated and administered in unit-dosage forms or multiple-dosage forms. Unit-dose forms as used herein refer to physically discrete units suitable for human and animal subjects and packaged individually as is known in the art. Each unit-dose contains a predetermined quantity of the therapeutically active compound sufficient to produce the desired therapeutic effect, in association with the required vehicle. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid compositions can, for example, be prepared by dissolving, dispersing, or otherwise mixing an active compound as defined above and optional adjuvants in a vehicle, such as, for example, water, saline, aqueous dextrose, glycerol, glycols, ethanol, and the like, to thereby form a solution or suspension, colloidal dispersion, emulsion or liposomal formulation. If desired, the composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975 or later editions thereof.

Dosage forms or compositions containing active ingredient in the range of 0.005% to 100% with the balance made up from vehicle or carrier may be prepared. Methods for preparation of these compositions are known to those skilled in the art. The contemplated compositions may contain 0.001%-100% active ingredient, in one aspect 0.1-95%, in another aspect 0.4-10%.

In certain aspects, the compositions are lactose-free compositions containing excipients that are well known in the art and are listed, for example, in the U.S. Pharmacopeia (USP) 25-NF20 (2002). In general, lactose-free compositions contain active ingredients, a binder/filler, and a lubricant in compatible amounts. Particular lactose-free dosage forms contain active ingredients, microcrystalline cellulose, pre-gelatinized starch, and magnesium stearate.

Further provided are anhydrous compositions and dosage forms comprising active ingredients, since water can facilitate the degradation of some compounds. For example, the addition of water (e.g., 5%) is widely accepted as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, NY, 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous compositions and dosage forms provided herein can be prepared using anhydrous or low moisture containing ingredients and low moisture or low humidity conditions.

An anhydrous composition should be prepared and stored such that its anhydrous nature is maintained. Accordingly, anhydrous compositions are generally packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Oral dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain aspects, the formulations are solid dosage forms such as for example, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an enteric coating; a film coating agent and modified release agent. Examples of binders include microcrystalline cellulose, methyl paraben, polyalkyleneoxides, gum tragacanth, glucose solution, acacia mucilage, gelatin solution, molasses, polyvinylpyrrolidine, povidone, crospovidones, sucrose and starch and starch derivatives. Lubricants include talc, starch, magnesium/calcium stearate, lycopodium and stearic acid. Diluents include, for example, lactose, sucrose, trehalose, lysine, leucine, lecithin, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water-soluble FD and C dyes, mixtures thereof, and water insoluble FD and C dyes suspended on alumina hydrate and advanced coloring or anti-forgery color/opalescent additives known to those skilled in the art. Sweetening agents include sucrose, lactose, mannitol and artificial sweetening agents such as saccharin and any number of spray dried flavors. Flavoring agents include natural flavors extracted from plants such as fruits and synthetic blends of compounds which produce a pleasant sensation or mask unpleasant taste, such as, but not limited to peppermint and methyl salicylate. Wetting agents include propylene glycol monostearate, sorbitan monooleate, diethylene glycol monolaurate and polyoxyethylene lauryl ether. Enteric-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate. Modified release agents include polymers such as the Eudragit® series and cellulose esters.

The compound, or derivative thereof, can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H₂ blockers, and diuretics. The active ingredient is a compound or derivative thereof as described herein. Higher concentrations, up to about 98% by weight of the active ingredient may be included.

In all aspects, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Vehicles used in elixirs include solvents. Syrups are concentrated aqueous solutions of a sugar, for example, sucrose, and may contain a preservative. An emulsion is a two-phase system in which one liquid is dispersed in the form of small globules throughout another liquid. Carriers used in emulsions are non-aqueous liquids, emulsifying agents and preservatives. Suspensions use suspending agents and preservatives. Acceptable substances used in non-effervescent granules, to be reconstituted into a liquid oral dosage form, include diluents, sweeteners and wetting agents. Acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water-soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example, propylene carbonate, vegetable oils or triglycerides, is in some aspects encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a liquid vehicle, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or polyalkylene glycol, including, but not limited to, 1,2-dimethoxyethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including an acetal. Alcohols used in these formulations are any water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

Parenteral administration, in some aspects, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Vehicles used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (Tween® 80). A sequestering or chelating agent of metal ions includes EDTA. Carriers also include ethyl alcohol, polyethylene glycol and propylene glycol for water miscible vehicles; and sodium hydroxide, hydrochloric acid, citric acid or lactic acid for pH adjustment.

The concentration of compound is adjusted so that an injection provides an effective amount to produce the desired pharmacological effect. The exact dose depends on the age, weight, body surface area and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another aspect is a sterile aqueous or oily solution or suspension containing an active material injected as necessary to produce the desired pharmacological effect.

Injectables are designed for local and systemic administration. In some aspects, a therapeutically effective dosage is formulated to contain a concentration of at least about 0.01% w/w up to about 90% w/w or more, in certain aspects more than 0.1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; 6,699,500 and 6,740,634. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain aspects, the agent may be administered using intravenous infusion, an implantable osmotic pump, a transdermal patch, liposomes, or other modes of administration. In some aspects, a pump may be used (see, Sefton, CRC Crit. Ref Biomed. Eng. 14:201 (1987); Buchwald et al., Surgery 88:507 (1980); Saudek et al., N. Engl. J. Med. 321:574 (1989)). In other aspects, polymeric materials can be used. In other aspects, a controlled release system can be placed in proximity of the therapeutic target, i.e., thus requiring only a fraction of the systemic dose (see, e.g., Goodson, Medical Applications of Controlled Release, vol. 2, pp. 115-138 (1984)). In some aspects, a controlled release device is introduced into a subject in proximity of the site of inappropriate immune activation or a tumor. Other controlled release systems are discussed in the review by Langer (Science 249:1527-1533 (1990)). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a derivative thereof, in a suitable solvent. The solvent may contain an excipient which improves the stability or other pharmacological component of the powder or reconstituted solution, prepared from the powder. Excipients that may be used include, but are not limited to, an antioxidant, a buffer and a bulking agent. In some aspects, the excipient is selected from dextrose, sorbitol, fructose, corn syrup, xylitol, glycerin, glucose, sucrose and other suitable agent. The solvent may contain a buffer, such as citrate, sodium or potassium phosphate or other such buffer known to those of skill in the art at, at about neutral pH. Subsequent sterile filtration of the solution followed by lyophilization under standard conditions known to those of skill in the art provides the desired formulation. In some aspects, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carriers. The precise amount depends upon the selected compound. Such amount can be empirically determined.

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in some aspects, have mass median geometric diameters of less than 5 microns, in other aspects less than 10 microns.

Oral inhalation formulations of the compounds or derivatives suitable for inhalation include metered dose inhalers, dry powder inhalers and liquid preparations for administration from a nebulizer or metered dose liquid dispensing system. For both metered dose inhalers and dry powder inhalers, a crystalline form of the compounds or derivatives is the preferred physical form of the drug to confer longer product stability.

In addition to particle size reduction methods known to those skilled in the art, crystalline particles of the compounds or derivatives can be generated using supercritical fluid processing which offers significant advantages in the production of such particles for inhalation delivery by producing respirable particles of the desired size in a single step. (e.g., International Publication No. WO2005/025506). A controlled particle size for the microcrystals can be selected to ensure that a significant fraction of the compounds or derivatives is deposited in the lung. In some aspects, these particles have a mass median aerodynamic diameter of about 0.1 to about 10 microns, in other aspects, about 1 to about 5 microns and still other aspects, about 1.2 to about 3 microns.

Inert and non-flammable HFA propellants are selected from HFA 134a (1,1,1,2-tetrafluoroethane) and HFA 227e (1,1,1,2,3,3,3-heptafluoropropane) and provided either alone or as a ratio to match the density of crystal particles of the compounds or derivatives. A ratio is also selected to ensure that the product suspension avoids detrimental sedimentation or cream (which can precipitate irreversible agglomeration) and instead promote a loosely flocculated system, which is easily dispersed when shaken. Loosely fluctuated systems are well regarded to provide optimal stability for pMDI canisters. As a result of the formulation's properties, the formulation contained no ethanol and no surfactants/stabilizing agents.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other excipients can also be administered.

For nasal administration, the preparation may contain an esterified phosphonate compound dissolved or suspended in a liquid carrier, in particular, an aqueous carrier, for aerosol application. The carrier may contain solubilizing or suspending agents such as propylene glycol, surfactants, absorption enhancers such as lecithin or cyclodextrin, or preservatives.

Solutions, particularly those intended for ophthalmic use, may be formulated as 0.01%-10% isotonic solutions, pH about 5-7.4, with appropriate salts.

Other routes of administration, such as transdermal patches, including iontophoretic and electrophoretic devices, and rectal administration, are also contemplated herein.

Transdermal patches, including iontophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433 and 5,860,957.

For example, dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one aspect, is about 2 to 3 gm. Tablets and capsules for rectal administration are manufactured using the same substance and by the same methods as for formulations for oral administration.

The compounds provided herein, or derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In some aspects, liposomal suspensions, including tissue-targeted liposomes, such as tumor-targeted liposomes, may also be suitable as carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down phosphatidyl choline and phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The compounds or derivatives may be packaged as articles of manufacture containing packaging material, a compound or derivative thereof provided herein, which is effective for treatment, prevention or amelioration of one or more symptoms of the diseases or disorders, supra, within the packaging material, and a label that indicates that the compound or composition or derivative thereof, is used for the treatment, prevention or amelioration of one or more symptoms of the diseases or disorders, supra.

The articles of manufacture provided herein contain packaging materials. Packaging materials for use in packaging products are well known to those of skill in the art. See, e.g., U.S. Pat. Nos. 5,323,907, 5,052,558 and 5,033,252. Examples of packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder described herein.

### Dosages

For use to treat or prevent infectious disease, the compounds described herein, or pharmaceutical compositions thereof, are administered or applied in a therapeutically effective amount. In human therapeutics, the physician will determine the dosage regimen that is most appropriate according to a preventive or curative treatment and according to the age, weight, stage of the disease and other factors specific to the subject to be treated. The amount of active ingredient in the formulations provided herein, which will be effective in the prevention or treatment of an infectious disease will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the infection, the route of administration, as well as age, body, weight, response, and the past medical history of the subject.

Exemplary doses of a formulation include milligram or microgram amounts of the active compound per kilogram of subject (e.g., from about 1 microgram per kilogram to about 50 milligrams per kilogram, from about 10 micrograms per kilogram to about 30 milligrams per kilogram, from about 100 micrograms per kilogram to about 10 milligrams per kilogram, or from about 100 micrograms per kilogram to about 5 milligrams per kilogram).

In some aspects, a therapeutically effective dosage should produce a serum concentration of active ingredient of from about 0.001 ng/ml to about 50-200 μg/ml. The compositions, in other aspects, should provide a dosage of from about 0.0001 mg to about 70 mg of compound per kilogram of body weight per day. Dosage unit forms are prepared to provide from about 0.01 mg, 0.1 mg or 1 mg to about 500 mg, 1000 mg or 5000 mg, and in some aspects from about 10 mg to about 500 mg of the active ingredient or a combination of essential ingredients per dosage unit form. A suitable, non-limiting example of a dosage of a disclosed compound according to the present disclosure may be from about 1 ng/kg to about 5000 mg/kg. In some aspects, the dosage may be in the range of 0.0001 mg/kg/day to 0.0010 mg/kg/day, 0.0010 mg/kg/day to 0.010 mg/kg/day, 0.010 mg/kg/day to 0.10 mg/kg/day, 0.10 mg/kg/day to 1.0 mg/kg/day, 1.00 mg/kg/day to about 200 mg/kg/day, 200 mg/kg/day to about 5000 mg/kg/day. For example, the dosage may be about 1 mg/kg/day to about 100 mg/kg/day, such as, e.g., 2-10 mg/kg/day, 10-50 mg/kg/day, or 50-100 mg/kg/day. The dosage can also be selected from about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1 100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, 2300 mg/kg, 2400 mg/kg, 2500 mg/kg, 2600 mg/kg, 2700 mg/kg, 2800 mg/kg, 2900 mg/kg, 3000 mg/kg, 3500 mg/kg, 4000 mg/kg, or 5000 mg/kg. In certain aspects, the dosage is about 1, 1.5, 2, 2.5, 3, 3.5, 4, 4.5, 5, 5.5, 6, 6.5, 7, 7.5, 8, 8.5, 9, 9.5, or 10 mg/kg. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

The active ingredient may be administered at once or may be divided into a number of smaller doses to be administered at intervals of time. It is understood that the precise dosage and duration of treatment is a function of the disease being treated and may be determined empirically using known testing protocols or by extrapolation from in vivo or in vitro test data or subsequent clinical testing. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions and that the concentration ranges set forth herein are exemplary only and are not intended to limit the scope or practice of the claimed compositions.

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

For systemic administration, a therapeutically effective dose can be estimated initially from in vitro assays. For example, a dose can be formulated in animal models to achieve a circulating concentration range that includes the IC₅₀ as determined in cell culture (i.e., the concentration of test compound that is lethal to 50% of a cell culture), or the IC₁₀₀ as determined in cell culture (i.e., the concentration of compound that is lethal to 100% of a cell culture). Such information can be used to more accurately determine useful doses in humans.

Initial dosages can also be estimated from in vivo data (e.g., animal models) using techniques that are well known in the art. One of ordinary skill in the art can readily optimize administration to humans based on animal data.

Alternatively, initial dosages can be determined from the dosages administered of known agents by comparing the IC₅₀, MIC and/or I₁₀₀ of the specific compound disclosed herein with that of a known agent and adjusting the initial dosages accordingly. The optimal dosage may be obtained from these initial values by routine optimization

In cases of local administration or selective uptake, the effective local concentration compound used may not be related to plasma concentration. One of skill in the art will be able to optimize therapeutically effective local dosages without undue experimentation.

Ideally, a therapeutically effective dose of the compounds described herein will provide therapeutic benefit without causing substantial toxicity. Toxicity of compounds can be determined using standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in subjects. The dosage of the compounds described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition (See, e.g., Fingl et al., 1975, In: The Pharmacological Basis of Therapeutics, Ch. 1, p. 1).

The therapy may be repeated intermittently. In certain aspects, administration of the same formulation provided herein may be repeated and the administrations may be separated by at least 1 day, 2 days, 3 days, 5 days, 10 days, 15 days, 30 days, 45 days, 2 months, 75 days, 3 months, or 6 months.

### Methods of Use of the Compounds and Compositions

Methods of treating, preventing, or ameliorating symptoms of medical disorders such as, for example, bacterial infections and diseases including gram positive bacterial infections, skin infection, endocarditis, osteomyelitis or pneumonia, HIV infection, cancer, Alzheimer's disease and coronavirus infection with a compound of structural Formula (I) are provided:

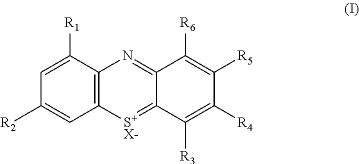

or pharmaceutically acceptable salts, solvates or hydrates thereof wherein: R₁ is H, —NR₇R₈, —OR₉, alkyl, substituted alkyl, alkenyl, substituted alkenyl, alkynyl, substituted alkynyl, heteroalkyl, substituted heteroalkyl or halo; R₂ is —H, —NR₁₀R₁₁ or

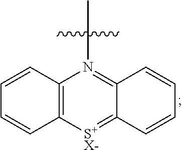

R₃ is —H, halo, —OR₁₂, alkyl or substituted alkyl; R₄ is —H, —NR₁₃R₁₄ or

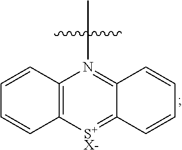

R₅ is —H or halo, —OR₁₅, alkyl or substituted alkyl; R₆ is —H, —NR₁₆R₁₇, —OR₁₅, alkyl, substituted alkyl, alkenyl, substituted alkenyl, alkynyl, substituted alkynyl, heteroalkyl, substituted heteroalkyl or halo; R₇ and R₈ are independently alkyl, substituted alkyl, alkenyl, substituted alkenyl, alkynyl, substituted alkynyl, heteroalkyl, substituted heteroalkyl or taken together with the atom to which they are bonded form a cycloheteroalkyl, substituted cycloheteroalkyl, cycloheteroalkenyl, substituted cycloheteroalkenyl, bicycloheteroalkyl, substituted bicycloheteroalkyl, spirocycloheteroalkyl or a substituted spirocycloheteroalkyl ring; R₁₀ and R₁₁ are independently alkyl, substituted alkyl, alkenyl, substituted alkenyl, alkynyl, substituted alkynyl, heteroalkyl, substituted heteroalkyl or taken together with the atom to which they are bonded form a cycloheteroalkyl, substituted cycloheteroalkyl, cycloheteroalkenyl, substituted cycloheteroalkenyl, bicycloheteroalkyl, substituted bicycloheteroalkyl, spirocycloheteroalkyl or a substituted spirocycloheteroalkyl ring; R₁₆ and R₁₇ are independently alkyl, substituted alkyl, alkenyl, substituted alkenyl, alkynyl, substituted alkynyl, heteroalkyl, substituted heteroalkyl or taken together with the atom to which they are bonded form a cycloheteroalkyl, substituted cycloheteroalkyl, cycloheteroalkenyl, substituted cycloheteroalkenyl, bicycloheteroalkyl, substituted bicycloheteroalkyl, spirocycloheteroalkyl or a substituted spirocycloheteroalkyl ring; R₉, R₁₂, R₁₅ and R₁₈ are independently alkyl, substituted alkyl, alkenyl, substituted alkenyl, alkynyl, substituted alkynyl, aryl, substituted aryl, heteroaryl, substituted aryl, heteroalkyl or substituted heteroalkyl; X— is a pharmaceutically acceptable salt; provided that when R₂ and R₄ both form a C₅-C₇ cycloheteroalkyl or C₅-C₇ substituted cycloheteroalkyl ring that R₁ is not H when R₆ is t-butyl, R₁ is not H or methyl when R₆ is H except when R₅ is —CH₂Boc or —OMe, R₁ is not methyl or ethyl when R₆ is ethyl; provided that none of R₁₀, R₁₁, R₁₃ or R₁₄ are alkyl or substituted alkyl when R₁ and R₆ are independently —H, alkyl, alkenyl or haloalkyl if either R₁₀ and R₁₁ or R₁₃ and R₁₄ form a C₅-C₇ cycloheteroalkyl or C₅-C₇ substituted cycloheteroalkyl ring; provided that at least one of R₁ and R₆ is not —H when one of R₂ and R₄ is —H; and provided that when R₂ is —NR₁₀R₁₁ and R₄ is —NR₁₃R₁₄ and any of R₁₀, R₁₁, R₁₃ or R₁₄ are substituted alkyl that none of R₁₀, R₁₁, R₁₃ or R₁₄ are substituted with —OR₁₉, —SR₂₀ or —NR₂₁ where R₁₉, R₂₀ or R₂₁ are independently alkyl, alkenyl or aryl.

In practicing the methods, therapeutically effective amounts of the compounds or compositions, described herein, supra, are administered to the patient with the disorder or condition.

The compounds provided in the present disclosure can be used to treat or prevent bacterial infections caused by Staphylococcus aureus, Escherichia coli, Acinetobacter baumannii, Enterococcus faecalis, Staphylococcus epidermidis, Streptococcus pyogens, Streptococcus pneumoniae, or Clostridium difficile. The bacterial strain can be an antibiotic sensitive strain, or an antibiotic aspect resistant strain. aspect Resistant strains include for example MRSA ATCC43300, macrolide resistant S. aureus, vancomycin resistant E. faecium, ciprofloxacin resistant E. faecium, tetracycline resistant S. aureus, MDR S. aureus, or any ampicillin resistant, chloramphenicol resistant or kanamycin resistant bacterial strains. The bacteria can be a gram-positive bacteria or a gram-negative bacteria.

Examples of infection caused by the bacteria include, but are not limited to, surgical wound infection, urinary tract infection, bloodstream infection (sepsis), pneumonia (hospital-acquired or community-acquired), diabetic foot infection, and skin infection such as cellulites, boils, abscesses, sty, carbuncles, and impetigo.

Treatment or prevention of bacterial infection can comprise administering the disclosed compounds to a subject that has an infection, or has a symptom of an infection, or has a predisposition toward such infection, with the purpose to cure, heal, alleviate, relieve, alter, remedy, ameliorate, improve, or affect the infection, the symptoms of the infection, or the predisposition toward the infection. Treatment can result in killing or inactivating the bacteria. Treatment can inhibit biofilm formation or reduce the bacterial load in the subject.

The present disclosure also provides a method for treating an HIV infection and/or AIDS. The HIV infection can be an HIV-1 infection or an HIV-2 infection. The HIV infection can be a strain A, B, C, D, F, G, H, J, K, M, N, O or P infection.

In some aspects, treatment of HIV infection can be reduction in the severity or duration of one or more HIV- or AIDS-related symptoms, inhibition, decrease, reduction, or otherwise reduce the severity of an HIV infection, delay in the onset of AIDS, and/or inactivation of HIV. Signs or symptoms of the initial stages of HIV infection include fever, swollen lymph nodes, sore throat, rash, muscle pain, malaise, and mouth and esophageal sores. AIDS, the final stage of HIV infection, may present with symptoms of various opportunistic infections, as is well-known in the art. Symptoms at this stage may include unexplained weight loss, recurring respiratory tract infections, prostatitis, skin rashes, and oral ulcerations.

In some aspects, the present disclosure provides a method for treating or preventing cancer. The method comprises administering to a subject in need thereof, a therapeutically effective amount of one or more of the disclosed compounds. As it will be recognized by individuals skilled in the art, cancer as used throughout the instant disclosure may be one or more neoplasm or cancer. The neoplasm may be malignant or benign, the cancer may be primary or metastatic; the neoplasm or cancer may be early stage or late stage. Non-limiting examples of neoplasms or cancers that may be treated include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood).

In some aspects, treatment of cancer can be inhibition of cancer progression and/or metastases, inhibition of an increase in tumor volume, a reduction in tumor volume, a reduction in tumor growth, an eradication of a tumor and/or cancer cell, or any combinations thereof. A disclosed treatment can also result in a prolonging survival of a subject or improvement in the prognosis of the subject.

### Combination Therapy

The compounds and compositions disclosed herein may also be used in combination with one or more other active ingredients. In certain aspects, the compounds may be administered in combination, or sequentially, with another therapeutic agent. Such other therapeutic agents include those known for treatment, prevention, or amelioration of one or more symptoms associated with a variety of bacterial infections, including gram positive bacterial infections, skin infection, endocarditis, osteomyelitis or pneumonia, HIV infection, cancer, Alzheimer's disease and coronavirus infection. Many such therapeutic agents are known in the art.

It should be understood that any suitable combination of the compounds and compositions provided herein with one or more of the above therapeutic agents and optionally one or more further pharmacologically active substances are considered to be within the scope of the present disclosure. In some aspects, the compounds and compositions provided herein are administered prior to or subsequent to the one or more additional active ingredients.

Finally, it should be noted that there are alternative ways of implementing the present invention. Accordingly, the present aspects are to be considered as illustrative and not restrictive, and the invention is not to be limited to the details given herein but may be modified within the scope and equivalents of the appended claims.

All publications and patents cited herein are incorporated by reference in their entirety.

The following examples are provided for illustrative purposes only and are not intended to limit the scope of the invention.

### EXAMPLES

Scheme 1 illustrates the preparation of compound 1.

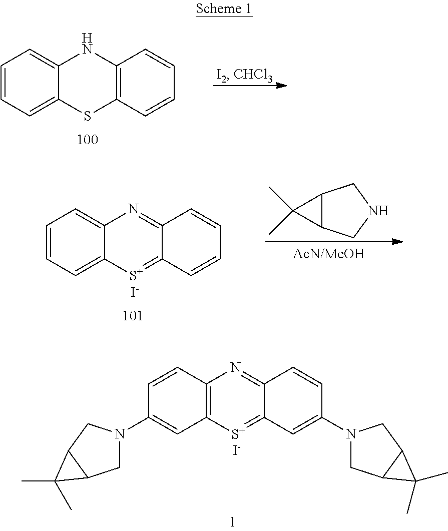

### Phenothiazin-5-ium Tetraiodide Hydrate (101)

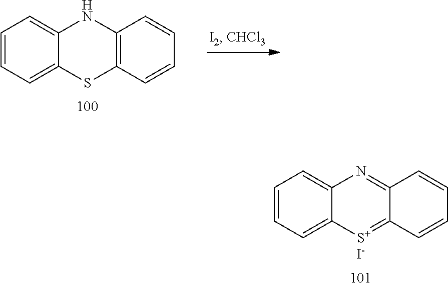 [SMILES: c1ccc2c(c1)Nc1ccccc1S2 ; c1ccc2[s+]c3ccccc3nc2c1 ; [I-]]

A solution of phenothiazine (100) (4.98 g, 25 mmol) in anhydrous chloroform (50 ml) was stirred at 5° C. and the solution of iodine (12.7 g, 50 mmol) in CHCl₃ (250 ml) was added dropwise over 4 h. The resulting dark solution was stirred for an additional 3 h at 5° C. and monitored by TLC. After the disappearance of the starting material, the resulting precipitate was filtered, washed with a copious amount of chloroform and dried overnight in vacuo to afford a dark solid (101) (13.9 g, 74%).

### Example 1: 3,7-Di(6,6-dimethyl-3-azabicyclo[3.1.0]hexan-N-yl)phenothiazinium iodide (1)

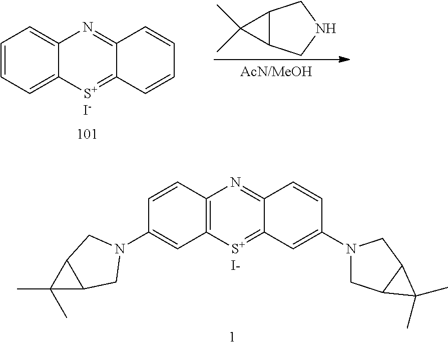

A solution of phenothiazin-5-ium tetraiodide hydrate (101) (3.0 g, 4.0 mmol) in a mixture of acetonitrile/methanol (50 ml) and 6,6-dimethyl-3-azabicyclo[3.1.0]hexane (1.11 g, 10 mmol) was stirred for 1 h at room temperature. The resulting mixture was concentrated to dryness and purified by reverse phase flash chromatography using an acetonitrile-water gradient to provide 3,7-Di(6,6-dimethyl-3-azabicyclo[3.1.0]hexan-N-yl)phenothiazinium iodide (1) (1.2 g, 53%).

Scheme 2 illustrates the preparation of compound 30.

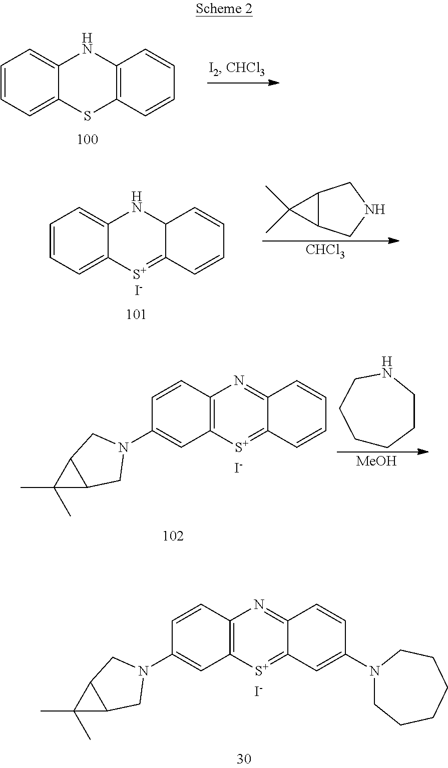

### 3-(6,6-dimethyl-3-azabicyclo[3.1.0]hexan-N-yl)phenothiazinium iodide (102)

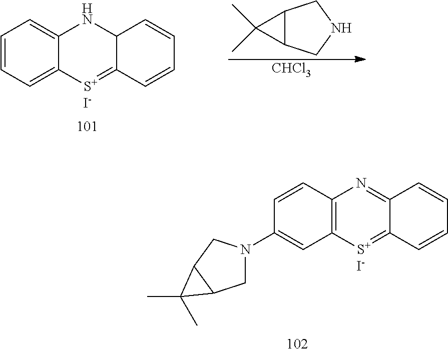

A solution of phenothiazin-5-ium tetraiodide hydrate (1) prepared as previously described, supra, (220 mg, 0.3 mmol) in chloroform (5 ml) and 6,6-dimethyl-3-azabicyclo[3.1.0]hexane (33 mg, 0.3 mmol) was stirred for 1 h at room temperature. The resulting mixture was concentrated to dryness to provide compound 102 and used without purification.

### Example 2: 3-[6,6-dimethyl-3-azabicyclo[3.1.0]hexan-N-yl]-7-(azepan-1-yl) phenothiazinium iodide (30)

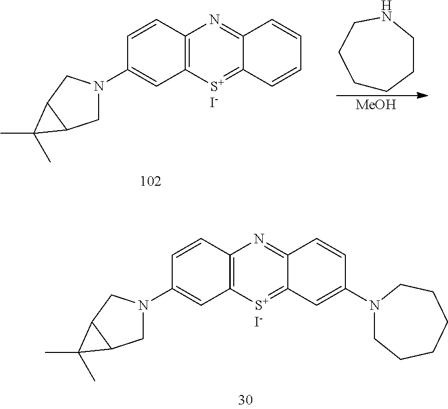 [SMILES: "MeOH" ; CC1(C)C2CN(c3ccc4nc5ccccc5[s+]c4c3)CC21 ; [I-] ; C1CCCNCC1 ; CC1(C)C2CN(c3ccc4nc5ccc(N6CCCCCC6)cc5[s+]c4c3)CC21 ; [I-]]

A solution of 3-[6,6-dimethyl-3-azabicyclo[3.1.0]hexan-N-yl]phenothiazine-5-ium triiodide (102) in methanol (5 ml) and azepane (100 mg, 1.0 mmol) was stirred for 1 h at room temperature. The resulting mixture was concentrated to dryness and purified by reverse phase flash chromatography to provide compound 30 (47 mg, 30%).

Scheme 3 illustrates another preparation of compound 1

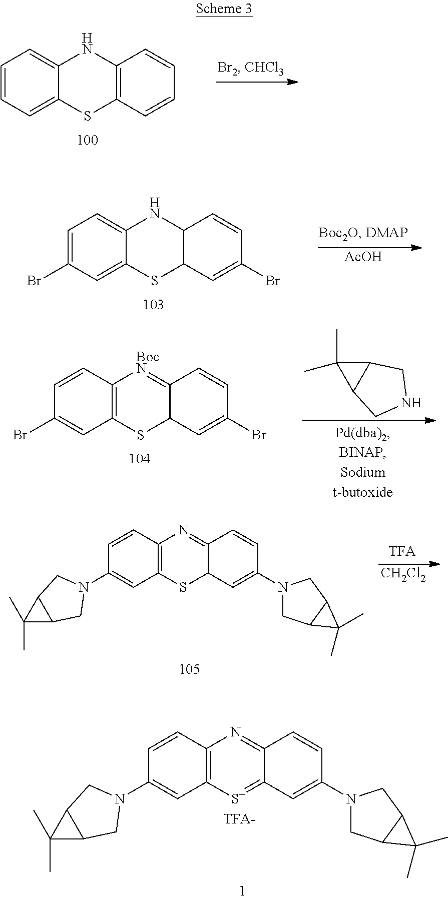

### 3,7-dibromo-10H-phenothiazine (103)

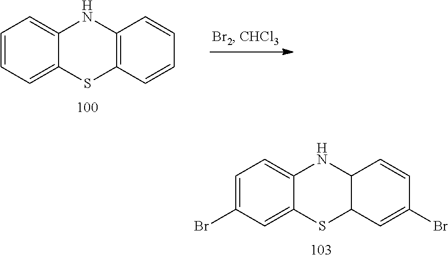 [SMILES: c1ccc2c(c1)Nc1ccccc1S2 ; BrC1=CC2Sc3cc(Br)ccc3NC2C=C1]

Phenothiazine (100) (1.23 g, 6.2 mmol) was dissolved in acetic acid (10 ml) and stirred at room temperature as a solution of bromine (2.96 g, 0.95 ml, 18.5 mmol) in acetic acid (50 ml) was added. The mixture was allowed to stir overnight and then Na₂SO₃ (1.56 g, 12.4 mmol) and water (2 ml) were added. The mixture was stirred at room temperature for 3 h and poured into 100 ml of ice-water contained NaOH (1.0 g, 25 mmol). The mixture was stirred overnight and filtered to provide compound 103 (1.62 g, 73%) as a light green solid. 3,7-dibromo-10-Boc-phenothiazine (104)

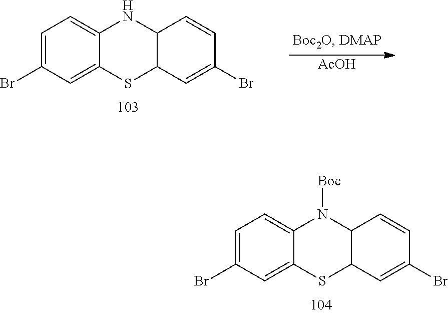 [SMILES: "AcOH" ; BrC1=CC2Sc3cc(Br)ccc3NC2C=C1 ; CC(C)(C)OC(=O)N1c2ccc(Br)cc2SC2C=C(Br)C=CC21]

3,7-dibromo-10H-phenothiazine (103) (1.6 g, 4.5 mmol) was suspended in AcN (20 ml) and Boc₂O (2.94 g, 13.5 mmol) and DMAP (0.55 g, 4.5 mmol) were added. The mixture was warmed to 50° C. After 5 min the starting material was dissolved in solvent, CO₂ was eliminated, and solid material formed. After 1 h the reaction mixture was cooled to room temperature and the solid was filtered off and dried in air to provide compound 104 ((1.65 g, 80%). 3,7-di(6,6-dimethyl-3-azabicyclo[3.1.0]hexan-N-yl)-10-Boc-phenothiazine (105)

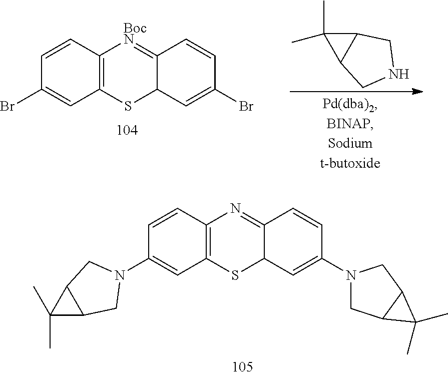

To a stirred solution of 3,7-dibromo-10-Boc-phenothiazine (104) (1.7 g, 3.74 mmol) in xylene were added 20 ml of Pd(dba)₂ (80 mg, 0.14 mmol), BINAP (70 mg, 0.11 mmol), sodium-t-butoxide (1.8 g, 18.7 mmol) and 6,6-dimethyl-3-azabicyclo[3,1,0]hexane (2.1 g, 18.7 mmol). The mixture was refluxed for 24 h, cooled, filtered, solvent was removed. The residue was purified by flash chromatography (hexane-ethyl acetate) purification to provide compound 105 (1.2 g, 63%).

### Example 3: 3,7-di(6,6-dimethyl-3-azabicyclo[3.1.0]hexan-N-yl)phenothiazin-5-ium trifluoroacetate (1)

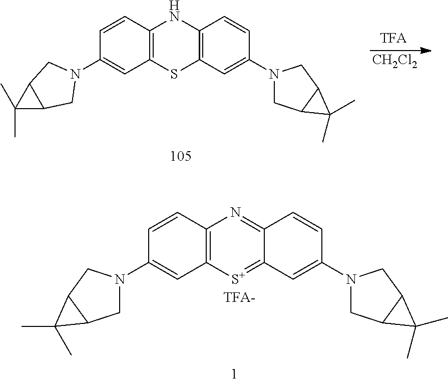

To a solution 3,7-di(6,6-dimethyl-3-azabicyclo[3.1.0]hexan-N-yl)-10-Boc-phenothiazine (1.2 g, 2.3 mmol) in dichloromethane (10 ml) TFA (4 mL) was added. The reaction mixture was stirred at room temperature for 3 h. The solvent was removed and the residue was purified by reverse phase flash chromatography to provide compound 1 (900 mg, 75%).

MS and HPLC data for compounds 1-150

MS and HPLC data for compounds 1-150 are shown in Table 2

TABLE 2

| Compound | HPLC purity | LCMS (M + 1) |
|---|---|---|
| 1 | >95% | 416.61 |
| 2 | >95% | 432.47 |
| 3 | >95% | 360.51 |
| 4 | >95% | 485.72 |
| 6 | >95% | 525.79 |
| 7 | >95% | 445.66 |
| 8 | >95% | 545.77 |
| 9 | >95% | 451.06 |
| 10 | >95% | 472.2 |
| 11 | >95% | 444.67 |
| 12 | >95% | 458.7 |
| 13 | >95% | 452.59 |
| 14 | >95% | 462.66 |
| 15 | >95% | 451.06 |
| 16 | >95% | 436.65 |
| 17 | >95% | 416.61 |
| 18 | >95% | 464.64 |
| 19 | >95% | 533.75 |
| 20 | >95% | 405.59 |
| 21 | >95% | 382.53 |
| 22 | v | 392.59 |
| 23 | >95% | 445.66 |
| 24 | >95% | 465.24 |
| 25 | >95% | 354.47 |
| 26 | >95% | 396.62 |
| 27 | >95% | 365.53 |
| 28 | >95% | 392.59 |
| 29 | >95% | 308.53 |
| 31 | >95% | 350.51 |
| 32 | >95% | 491.68 |
| 33 | >95% | 338.5 |
| 34 | >95% | 380.58 |
| 35 | >95% | 310.45 |
| 36 | >95% | 366.55 |
| 37 | >95% | 295.43 |
| 38 | >95% | 336.84 |
| 39 | >95% | 392.59 |
| 40 | >95% | 364.54 |
| 41 | >95% | 378.57 |
| 42 | >95% | 405.37 |
| 43 | >95% | 354.47 |
| 44 | >95% | 372.46 |
| 45 | >95% | 378.57 |
| 46 | >95% | 406.62 |
| 47 | >95% | 488.68 |
| 48 | >95% | 472.48 |
| 49 | >95% | 594.80 |
| 50 | >95% | 464.54 |
| 52 | >95% | 392.52 |
| 53 | >95% | 408.44 |
| 54 | >95% | 372.46 |
| 55 | >95% | 493.70 |
| 58 | >95% | 493.7 |
| 59 | >95% | 312.46 |
| 60 | >95% | 340.52 |
| 61 | >95% | 388.56 |
| 62 | >95% | 364.54 |
| 63 | >95% | 475.52 |
| 65 | >95% | 392.52 |
| 66 | >95% | 340.52 |
| 67 | >95% | 516.73 |
| 68 | >95% | 336.48 |
| 69 | >95% | 509.74 |
| 70 | >95% | 392.59 |
| 71 | >95% | 404.48 |
| 72 | >95% | 408.59 |
| 73 | >95% | 380.58 |
| 74 | >95% | 495.71 |
| 75 | >95% | 473.59 |
| 76 | >95% | 435.62 |
| 77 | >95% | 463.72 |
| 78 | >95% | 430.60 |
| 79 | >95% | 350.51 |
| 80 | >95% | 396.62 |
| 81 | >95% | 435.66 |
| 82 | >95% | 366.51 |
| 83 | >95% | 395.60 |
| 84 | >95% | 366.51 |
| 85 | >95% | 450.68 |
| 86 | >95% | 370.93 |
| 88 | >95% | 424.59 |
| 89 | >95% | 448.66 |
| 90 | >95% | 504.73 |
| 91 | >95% | 448.66 |
| 92 | >95% | 408.60 |
| 93 | >95% | 396.54 |
| 94 | >95% | 381.57 |
| 95 | >95% | 509.70 |
| 96 | >95% | 539.71 |
| 97 | >95% | 410.63 |
| 98 | >95% | 492.76 |
| 99 | >95% | 442.7 |
| 100 | >95% | 421.63 |
| 101 | >95% | 626.35 |
| 102 | >95% | 517.26 |
| 103 | >95% | 416.14 |
| 104 | >95% | 642.38 |
| 105 | >95% | 503.25 |
| 106 | >95% | 503.25 |
| 107 | >95% | 490.23 |
| 108 | >95% | 418.21 |
| 109 | >95% | 390.17 |
| 110 | >95% | 546.29 |
| 111 | >95% | 532.27 |
| 112 | >95% | 618.31 |
| 113 | >95% | 604.3 |
| 114 | >95% | 590.28 |
| 115 | >95% | 590.28 |
| 116 | >95% | 503.25 |
| 117 | >95% | 407.23 |
| 118 | >95% | 423.17 |
| 119 | >95% | 434.24 |
| 120 | >95% | 420.22 |
| 121 | >95% | 534.29 |
| 122 | >95% | 520.27 |
| 123 | >95% | 421.21 |
| 124 | >95% | 485.27 |
| 125 | >95% | 485.27 |
| 126 | >95% | 581.37 |
| 127 | >95% | 504.2 |
| 128 | >95% | 562.27 |
| 129 | >95% | 532.23 |
| 130 | >95% | 461.27 |
| 131 | >95% | 1235.6 |
| 132 | >95% | 470.13 |
| 133 | >95% | 408.21 |
| 134 | >95% | 418.21 |
| 135 | >95% | 618.31 |
| 136 | >95% | 332.12 |
| 137 | >95% | 338.14 |
| 138 | >95% | 538.25 |
| 139 | >95% | 394.21 |
| 140 | >95% | 594.31 |
| 141 | >95% | 396.17 |
| 142 | >95% | 566.28 |
| 143 | >95% | 400.1 |
| 144 | >95% | 1240.5 |
| 145 | >95% | 445.24 |
| 146 | >95% | 416.22 |
| 147 | >95% | 416.22 |
| 148 | >95% | 504.16 |
| 149 | >95% | 505.26 |
| 150 | >95% | 376.18 |
| 151 | >95 | 407.61 |
| 152 | >95% | 381.57 |

### Example 4: In Vitro Antibacterial Activity (MIC Assay)

Determination of minimum inhibitory concentration (MIC) against sensitive gram-positive bacteria like Staphylococcus aureus ATCC25923, Escherichia coli ATCC25922, Acinetobacter baumannii ATCC19606, Enterococcus faecalis ATCC29212, E. faecium ATCC700221, Staphylococcus epidermidis ATCC14990, Streptococcus pyogens ATCC49399, Streptococcus pneumoniae ATCC 49619, Clostridium difficile ATCC BAA18770 was conducted by serial dilution method in culture medium (Clinical Laboratory Standards Institute, Document M7-A7³. Methods for dilution antimicrobial susceptibility tests for bacteria that grow aerobically; approved standard-7th Edition, Wayne, Pa. Clinical Laboratory Standards Institute, 2006.)

In this method, an aliquot of 10 mM stock test compound from an 8-point series of 2-fold serial dilutions of compounds in 100% DMSO was added to wells in microdilution plates. The test organisms were prepared by adjusting the turbidity of actively growing broth cultures so that the final concentration of test organism after addition to wells with or without compounds was approximately 5×10⁵ CFU/mL. Following inoculation of the microdilution plates, the plates were incubated at 37° C. for 16-24 hr then scored for bacterial growth. The MIC is defined as the lowest concentration of test compound that completely inhibits the visible growth of the test organism or a redox fluorescent dye resazurin. Bacterial growth in wells will convert resazurin into resorufin. The amount of growth in the wells containing the test compound was compared with the amount of growth in the growth-control wells (no test compound used) in each plate, and with the amount of growth in the wells containing control drug (such as ciprofloxacin). The results for compound 1 are summarized in Table 1, below. In vitro susceptibility test of compound 1 showed potent activity against different species of gram-positive bacteria with MIC range of 0.25-0.5 μg/mL, with exception on E. faecalis showed 2 μg/mL (Table 3)

TABLE 3
Minimum Inhibitory Concentration (MIC) of compound against various bacteria.
MIC in μg/mL

| | | S. | S. | S. | E. | E. | | A. |
|---|---|---|---|---|---|---|---|---|
| | S. aureus | epidermidis | pyogens | pneumoniae | faecalis | faecium | E. coli | baumannii |
| | ATCC25923 | ATCC14990 | ATCC49399 | ATCC49619 | ATCC29212 | ATCC27270 | ATCC25922 | ATCC19606 |
| 1 | 0.25 | 0.5 | 0.25 | 0.25 | 2 | 0.5 | 4 | 2 |
| 116 | 1.60 | | | | | | 12.5 | 6.25 |
| 117 | 1.60 | | | | | | 25 | 12.5 |
| 118 | 6.25 | | | | | | >100 | >100 |
| 119 | 25.00 | | | | | | 100 | >100 |
| 120 | 100 | | | | | | >100 | >100 |
| 121 | 0.8 | | | | | | >100 | 100 |
| 122 | 0.8 | | | | | | 100 | 25 |
| 123 | 6.25 | | | | | | >100 | 100 |
| 124 | 6.25 | | | | | | 100 | 12.5 |
| 125 | 12.5 | | | | | | >100 | >100 |
| 126 | 100 | | | | | | >100 | >100 |
| 127 | >100 | | | | | | >100 | >100 |
| 128 | 100 | | | | | | >100 | >100 |
| 129 | 12.5 | | | | | | >100 | >100 |
| 130 | 6.25 | | | | | | 12.5 | 6.25 |
| 132 | 6.25 | | | | | | 100 | 12.5 |
| 133 | 12.5 | | | | | | >100 | 100 |
| 134 | >100 | | | | | | >100 | >100 |
| 135 | 6.25 | | | | | | >100 | >100 |
| 136 | 25.00 | | | | | | >100 | >100 |
| 137 | >100 | | | | | | >100 | >100 |
| 139 | >100 | | | | | | >100 | >100 |
| 140 | >100 | | | | | | >100 | >100 |
| 141 | 100 | | | | | | >100 | >100 |
| 142 | 25 | | | | | | >100 | >100 |
| 142 | >25 | | | | | | >100 | >100 |
| 145 | 1.6 | | | | | | 25 | 6.25 |
| 146 | 3.1 | | | | | | >100 | 25 |
| 147 | 1.6 | | | | | | 100 | 25 |
| 148 | 25 | | | | | | >100 | 100 |
| 150 | 1.6 | | | | | | 100 | 25 |

### Example 5: Minimum Bactericidal Concentration Assay

Minimum bactericidal activity of compounds was determined against sensitive gram-positive bacteria which includes Staphylococcus aureus ATCC25923, Enterococcus faecalis ATCC29212, E. faecium ATCC700221, Staphylococcus epidermidis ATCC14990, Streptococcus pyogens ATCC49399, Streptococcus pneumoniae ATCC 49619 by checking viability of bacteria in a MIC plate. The study was done based on CLSI document M26-A Wayne, PA: Clinical and laboratory standard institute: 1999.

After determination of a MIC in a broth system under standard conditions, measured aliquots of growth media may be sub-cultured quantitatively to solid media to assess bactericidal activity. To calculate the extent of killing at each antibiotic concentration, plates are incubated under appropriate conditions, colony counts are performed and results are compared to that of the growth control well. The accepted definition of the minimum bactericidal concentration (MBC) is the lowest concentration of test compound at which a 99.9% (3 log) or greater reduction in growth compared with the initial inoculum is observed. Minimum bactericidal concentration of compound 1 against all the gram-positive species was not exceeded >2× MIC indicting its bactericidal mode of killing. (Table 4).

TABLE 4
Minimum Bactericidal Concentration (MBC) of compound 1 against sensitive gram-positive bacteria.
MBC in μg/mL

| | | S. | S. | S. | E. | |
|---|---|---|---|---|---|---|
| | S. aureus | epidermidis | Pyogens | pneumoniae | faecalis | E. faecium |
| | ATCC25923 | ATCC14990 | ATCC49399 | ATCC49619 | ATCC29212 | ATCC27270 |
| Compound | 0.25 | 1 | 0.5 | 0.5 | 2 | 0.5 |
| 1 | | | | | | |

The results for selected compounds against E. Coli as EC50 are shown in Table 5 below.

TABLE 5

| Compound | Anti-bacterial Activity |
|---|---|
| Number | (E. coli MIC (uM)) |
| 1 | 1.6 |
| 2 | 1.6 |
| 3 | 1.6 |
| 4 | 3.1 |
| 5 | 6.25 |
| 6 | 6.3 |
| 7 | 6.3 |
| 8 | 6.3 |
| 9 | 12.5 |
| 10 | 6.25 |
| 11 | 6.25 |
| 12 | 6.25 |
| 13 | 12.5 |
| 14 | 6.25 |
| 15 | 12.5 |
| 17 | 3.1 |
| 18 | 12.5 |
| 19 | 12.5 |
| 20 | 0.8 |
| 21 | 1.6 |
| 22 | 1.6 |
| 23 | 1.6 |
| 24 | 1.6 |
| 25 | 3.1 |
| 26 | 6.25 |
| 27 | 12.5 |
| 28 | 12.5 |
| 29 | 25 |
| 30 | 0.8 |
| 31 | 1.6 |
| 32 | 6.25 |
| 33 | 1.6 |
| 34 | 1.6 |
| 35 | 12.5 |
| 36 | 1.6 |
| 37 | 6.3 |
| 38 | 6.3 |
| 39 | 3.1 |
| 40 | 1.6 |
| 41 | 1.6 |
| 42 | 1.6 |
| 43 | 3.1 |
| 44 | 6.3 |
| 45 | 0.8 |
| 46 | 3.1 |
| 47 | 3.1 |
| 48 | 3.1 |
| 49 | 1.6 |
| 50 | 1.6 |
| 51 | 6.3 |
| 52 | 12.5 |
| 53 | 12.5 |
| 54 | 25 |
| 57 | 12.5 |
| 61 | 25 |
| 62 | 6.3 |
| 64 | 6.3 |
| 65 | 3.1 |
| 68 | 6.3 |
| 72 | 25 |
| 73 | 100 |
| 74 | 6.3 |
| 77 | 3.1 |
| 80 | 25 |
| 83 | 100 |
| 93 | 100 |
| 97 | 25 |
| 99 | 6.3 |
| 100 | 25 |

### Example 6: Cross Resistance Test

MIC of the compounds was tested as mentioned above, against resistant strains that includes MRSA ATCC43300, macrolide resistant S. aureus, vancomycin resistant E. faecium, ciprofloxacin resistant E. faecium, tetracycline resistant S. aureus, MDR S. aureus, ampicillin resistant, chloramphenicol resistant and kanamycin resistant strains. Compound 1 showed potent in vitro activity against bacteria which are resistant to different class of antibiotics and MDR S. aureus with MIC range of 0.25-0.5 μg/mL which confirms no cross resistance. (Table 6).

TABLE 6
Activity of compound 1 against resistant bacteria in ‘X’ MIC
‘X’ MIC

| | Compound 1 |
|---|---|
| Sensitive strain in μg/mL | 0.25 |
| Methicillin resistant | 1X |
| Macrolide resistant | 1X |
| Vancomycin resistant | 1X |
| Ciprofloxacin resistant | 1X |
| Tetracycline resistant | 1X |
| Chloramphenicol resistant | 1X |
| Kanamycin resistant | 1X |
| MDR | 1/2X |

### Example 7: Single Step Resistance Frequency

Single step resistance frequency (FOR) was determined by Graham Bell and Craig MacLean, 2018, Trends Microbiol. 471-483. By growing S. aureus ATCC25923, MRSA ATCC43300, MDR S. aureus ATCC BAA44, Vancomycin resistant E. faecium (VRE), Macrolide resistant S. aureus ATCC BAA976 and Ciprofloxacin resistant E. faecium on 4× and 8× test compound containing media. Ratio of colonies formed on media over number of bacteria inoculated gives the resistance frequency. The frequency of spontaneous mutant resistant to one of the compound 1 was determined in duplicate experiments. A bacterial suspension (10⁹-10¹⁰ cfu/mL) of above-mentioned strains was inoculated (100 μL) onto MH2 agar plates containing 4×, 8× MIC of compound tested. After 48 h of incubation of the plates at 37° C. in ambient air, the colonies were counted. We confirmed that the isolated colonies corresponded to resistant bacteria by determination of their MICs using the broth microdilution method. Resistance was defined as an MIC that was ≥4× MIC for the parental strain. The frequency of single-step resistance to a given compound was calculated by the ratio of the number of confirmed resistant colonies to the total number of colonies obtained in a drug-free control. In vitro single step resistance frequency of compound 1 against sensitive and resistant gram-positive bacteria is less than 10⁻⁹ at 4× and 8× MIC concentration (Table 7).

TABLE 7
Single step resistance frequency of compound 1.

Compound 1

| Strains | at 4X | at 8X |
|---|---|---|
| MSSA ATCC 25923 | 2 × 10⁻⁹ | 2 × 10⁻¹⁰ |
| MRSA ATCC 43300 | 1.5 × 10⁻⁹ | 1.5 × 10⁻¹⁰ |
| Macrolide resistant S. aureus | 8 × 10⁻¹⁰ | 8 × 10⁻¹¹ |
| ATCC BAA976 | | |
| MDR S. aureus ATCC BAA44 | 5 × 10⁻¹⁰ | 5 × 10⁻¹¹ |
| Vancomycin resistant E. | 2 × 10⁻⁹ | 2 × 10⁻⁹ |
| faecium ATCC 700221 | | |
| Ciprofloxacin resistant E. | 2 × 10⁻⁹ | 2 × 10⁻⁹ |
| faecium | | |

Compound 1 demonstrated potent activity against 17 isolates of S. aureus/MRSA with MIC50 value 0.125 μg/mL. (Table-6)

TABLE 8
MIC50 of compound 1 against MRSA/S. aureus isolates in μg/mL

| | No of isolates | MIC50 |
|---|---|---|
| Compound 1 | 17 | 0.125 |

### Example 8: Time Kill Kinetics

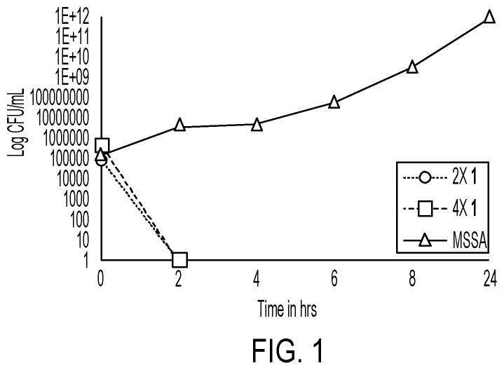

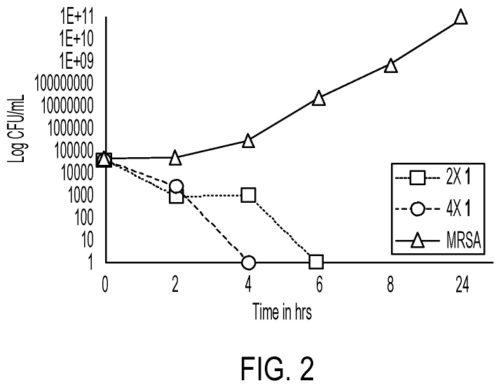

In vitro time kill kinetics study with compound 1 against S. aureus ATCCC 25923, MRSA ATCC43300 and MDR S. aureus BAA44 was done by the CLSI M26-A procedure. Overnight culture of testing strains was adjusted to obtain 5×10⁵ cfu/mL of a primary inoculum. The primary inoculum was split into 5 mL aliquots in 50 falcon tubes, one serving as an antibiotic-free growth control (containing 4% DMSO) and others receiving, respectively, compound 1 (in DMSO) at 2 times and 4 times respective MIC for the tested strain. Cultures were incubated for 24 h at 37° C. At 0, 2, 4, 8 and 24 h of incubation, a 100-200 μL aliquot was taken from each culture after shaking. Then, 50 μL of 10-fold serial dilutions in 1×PBS of each aliquot were plated onto Tryptic soya agar plates. CFU counts were determined after 24 h of incubation of the plates at 37° C. in ambient air. A significant bactericidal effect corresponded to a ≥3 log 10 reduction of the primary bacterial inoculum at any time of incubation. Time kill kinetics data of compound 1 shows that it is a bactericidal antibacterial agent kills both sensitive and resistant bacteria within 6 hrs of treatment (FIGS. 1, 2 and 3).

### Example 9: Post Antibiotic Effect

The post antibiotic effect was calculated using the equation PAE=T−C, where T represents the time (h) required for the viable count to increase ≥10-fold over the post-washing count in the presence of antibiotic and C represents the time required for the viable count to increase ≥10-fold over the post-washing count in the absence of antibiotic. The standard viable-cell plate count method described by Craig and Gudmundsson in Antibiotics in Laboratory Medicine, Lorian, V, ed. was used.

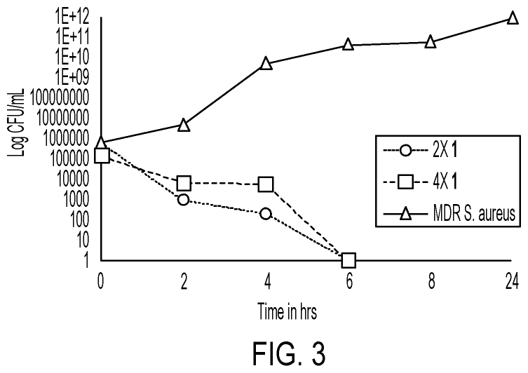

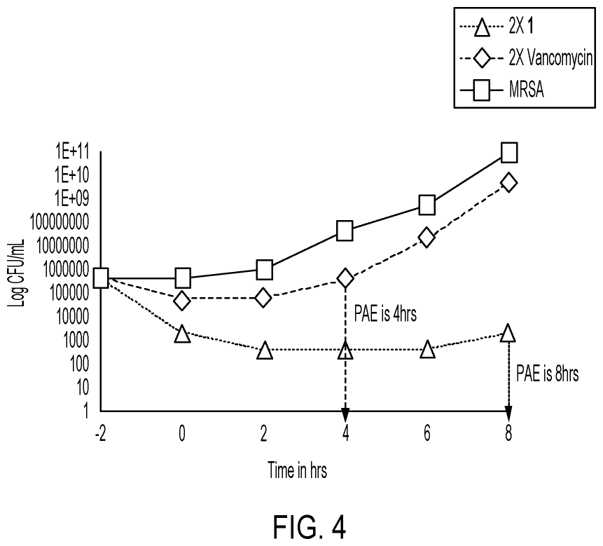

The starting cell concentration in the PAE should be between 1×10⁵ and 5×10⁵ cfu/ml. The liquid culture alone or with drug (at 1× or 2×MIC; tubes from step 8) was exposed for 1 hr, at 37° C. with shaking at 140 rpm. Following the incubation period, the drug was removed by centrifuging for 10 min at 5000 rpm, room temperature. The supernatant was discarded and the organism was resuspended in fresh, 37° C., drug-free medium. After the wash, 200 μl of sample (T=0 after washing) was removed and then at T=2, 4, 6, 8 and 24 hr and the sample was diluted ten-fold with sterile 1× PBS. A 10 μl aliquot of each dilution was placed onto individual TSA agar plates. The plates were incubated at 37° C. for 24 hr. After incubation, the colonies were counted and the results recorded. The data was plotted with time (hr) on the x-axis and colony forming units per ml (CFU/ml) on the y-axis. The post antibiotic effect after 2 hrs exposure of compound 1 was found to be ≥8 rs against MRSA and MDR (FIGS. 3 and 4).

### Example 10: Minimum Biofilm Inhibition Assay

The biofilm susceptibility assay and calculation of the percent reduction of AB were performed as described previously (Pettit et al., Antimicrob Agents Chem 2005, 49:2612-17). Biofilm formation was induced by growing MRSA on TSA containing 2% glucose at 37° C. for 24 hrs. After the incubation, culture was used to adjust the cell number to 1-5×10\hat{ }5 CFU/mL. In parallel, two-fold dilutions of test drug/antibiotic in DMSO/solvent was prepared separately in a PCR dilution plate. 100 μL of CFU adjusted 100 μL of culture was added to 96 well plate by leaving wells for only media control. After adding culture, 2-fold diluted compound in 100% DMSO was added to 96 well plate and plates were incubated another 16-20 hrs at 37° C., to check susceptibility of biofilm forming MRSA with test drug/antibiotic. After susceptibility incubation, Resazurin was added to check inhibition of biofilm forming cells. Compound 1 can inhibit biofilm forming MRSA at the concentration equivalent 1× MIC and (Table 9).

TABLE 9
Minimum biofilm inhibition assay (MBIC)
MRSA ATCC 43300

| | MIC | MBIC |
|---|---|---|
| Compound 1 | 0.25 | 0.25 |

### Example 11: Minimum Biofilm Eradication Assay

Biofilm was cultivated in TSB+2% glucose for 24 hours. Matured biofilm was treated for 24 hours by tested compounds diluted in Cation-Adjusted Muller-Hinton broth. The ability of compound/antibiotic was determined by checking viability of compound/antibiotic treated culture from 96 well plates as described by Christensen et al., J Clin Microbiol 1985; 22:996-1006.

Biofilms were formed in 96 well plate as described above and the plate was washed with 1×PBS to remove non-biofilm forming cells/non-adherent cells. Fresh CAMHb was added to the biofilm plate to which an aliquot of test compound from an 8-point series of 2-fold serial dilutions of compounds in 100% DMSO was added to the wells in the biofilm containing plates, which were incubated at 37° C. for 24 hrs to determine the eradication ability of compound. After incubation, the viability of bacteria in the biofilm plate treated with drug/antibiotic tested by resazurin addition or CFU enumeration. The lowest concentration of drug at which there is no bacterial viability was considered the MBEC concentration. Compound 1 showed the ability to invade S. aureus cells inside the biofilm and able to kill them under in vitro conditions, but at a high concentration. The biofilm eradication activity is equivalent to Linezolid, and better than Vancomycin (FIG. 3).

### Example 12: Activity Against NorA Efflux Pumps

Over expression of NorA efflux pump was induced by continuously exposing bacteria to sublethal concentration of EtBr as per the Isabel Couto et al., protocol (J. Antimicrob. Chemother. 2008, 504-513). MIC of compound 1 and Ciprofloxacin was determined to test whether MIC is affected due to efflux pumps overexpression. NorA efflux pump overexpression does not affect MIC of compound 1 but affects Ciprofloxacin MIC (Table 10).

TABLE 10
Compound 1 activity against NorA efflux pump overexpression

| | S. aureus | NorA overexpressing |
|---|---|---|
| | ATCC 25923 | S. aureus |
| Compound 1 | 0.25 | 0.25 |

### Example 13: Serum Effect on Compound 1 Antibacterial Activity

MIC in the presence of 20% and 40% FBS was determined by microbroth dilution method as described above. MIC in the presence of 20% and 40% serum was determined to access the effect of serum on the compound activity. Results showed that compound 1 showed reduced activity in the presence of serum. This may be due to compound 1 binding to protein. (Table 11).

TABLE 11
Serum effect on Compound 1 antibacterial activity

| | MIC in μg/mL against MRSA ATCC 43300 | | |
|---|---|---|---|
| | No serum | 20% serum | 40% serum |
| Compound 1 | 0.25 | 1 | 2 |

### Example 14: Pharmacokinetics and Organ Exposure

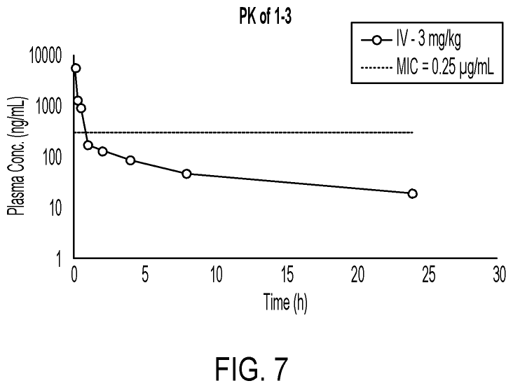

For the mouse plasma pharmacokinetic analyses, the study was conducted in BALB/c mice with single dose of IV-3 mg/kg. Blood samples were collected at 10 time points/dose (0.25, 0.5, 1, 2, 4, 6, 8, 10, 12, and 24 h post dose). At each time point, blood samples were collected from 4 mice from the retro-orbital plexus. A terminal blood collection method was employed for collecting blood samples for each time point in all PK studies. The plasma concentrations of compound 1 were estimated by a validated high-performance liquid chromatography tandem mass spectrometry (HPLC-MS/MS) method. The PK parameters were estimated by noncompartmental analysis. Organ exposure of compound 1 was done in mice by collecting different organs post dosing of 3 mg/kg though IV. The serum concentration-time curves of compound 1 in mice after administering a single dose of 3 mg/kg are shown in FIG. 7. The pharmacokinetic constants, AUC and Cₘₐₓ were found be more than 10× MIC with a Tₘₐₓ of 0.08 h. Compound 1 has half-life of 10 h with clearance in plasma being 17 mL/min/kg and volume of distribution of 7 L/kg (Table 10). Distribution in lungs and liver was found to be very high. Whereas, in kidney and heart the distribution is above the MIC and sufficient to achieve efficacy (Table 12).

TABLE 12
PK parameters of Compound 1 in mice
Pharmacokinetic Parameters

| Route/ | AUC | AUC | | | | Half- | | Plasma | Vss | Bio |
|---|---|---|---|---|---|---|---|---|---|---|
| Dose | (last) | (0 to ∞) | Cₘₐₓ | Tₘₐₓ | Kel | life | MRT | clearance | obs | availability |
| IV-3 | 2712 | 2984 | 5616 | 0.08 | 0.07 | 10 | 7 | 17 | 7 | — |

Dose—mg/kg, AUC—ng. h/mL, Cₘₐₓ-ng/mL, Tₘₐₓ-h, Kel—1/h, Half-life-h, MRT-h, Clearance—mL/min/kg, V_{d}-L/kg, Bioavailability-%. IV—Intravenous, IP— Intraperitoneal, PO—Per oral.

TABLE 13
Organ exposure of Compound 1 in ng at different interval of time.

| | | Time in hrs | | |
|---|---|---|---|---|
| Organ | | 0.5 | 2 | 24 |
| Concentration | Muscle | 154.5 | 324.5 | 168.33 |
| in ng | Lungs | 13003 | 4178 | 661 |
| | Kidney | 1817 | 2179 | 637 |
| | Heart | 1335 | 1951 | 821 |
| | Liver | 13897 | 11563 | 864 |
| | Brain | 81 | 56 | 30 |

### Example 15: Oncology Screens

All compounds mentioned as active were tested in Hennes. Select hits from the Hennes screen were sent for additional anti-cancer testing.

### Hennes

To screen compounds for oncology activity Hennes-20 cells were plated at low (500 cells/well) versus high (15,000 cells/well) densities and treated with DMSO (vehicle) or dose-titration of compounds. The rationale for this screen is that an intrinsically toxic compound should kill cells regardless of density, including in Hennes-20 cells. However, a compound that selectively triggers the arrest of proliferation will inhibit cell growth when plated at a low density will appear cytotoxic due to inhibition of cell growth when plated at a low density but will appear non-toxic to cells plated at a high density where the cells are approaching confluence and the readout detected by a cell viability assay is already close to the maximum.

To conduct the Hennes-20 screen, two 96 well plates were seeded with Hennes 20 cells in parallel where one was plated at a density of 500 cells/well and the other was plated at a density of 15,000 cells/well. 90 uL of minimum essential media was added to each well and plates were placed in a 37 degrees Celsius incubator for 24 hours. The next day, 10 ul of media containing dilutions of compound in DMSO were added to each plate. Six wells on each plate received 10 ul of media containing only DMSO. Each well was gently mixed 5 times with a 100 ul pipette. Plates were incubated at 37 degrees Celsius for 72 hours and then 10 uL of alamar Blue was added to each well. Wells were mixed 5 times then incubated at 37 degrees Celsius for 72 hours. Plates were then read at 530/590.

The rationale for the screen is as follows: Hennes-20 cells lack endogenous apoptosis. Cells plated at low density will proliferate and approach confluence in 72 hrs. A drug that arrests proliferation may have its proliferation blocking effect masked by the triggering of endogenous apoptosis. However, in the Hennes line, lacking endogenous apoptosis, such an arrest is manifest as a dose-dependent maintenance at low cell density. Since the high-density cells are plated close to confluence, the difference between Alamar Blue cell survival readings after 72 hrs reflects the ability of the drug to block proliferation in the low density-plated cells. In the high density-plated cells any diminution of Alamar Blue readout vs vehicle control is due to intrinsic toxicity, not proliferation arrest, since cells were already at confluence. Thus, a compound that appears toxic (low Alamar Blue readings after 72 hrs) on the low-density plate, but which appears non-toxic (high Alamar Blue readings after 72 hrs) on the high-density plate, must be arresting proliferation, since intrinsic compound toxicity should be observed at both low and high cell densities, not selectively on the low-density plate.

The results for selected compounds are shown in Table 15.

TABLE 15

| Compound Number | EC50 (uM) |
|---|---|
| 1 | 0.5 |
| 4 | 0.5 |
| 5 | <0.1 |
| 6 | 0.5 |
| 7 | <0.2.5 |
| 8 | <2.5 |
| 9 | 0.5 |
| 10 | <0.5 |
| 11 | <0.5 |
| 12 | <0.5 |
| 13 | 2.5 |
| 14 | <0.5 |
| 15 | 2.5 |
| 20 | 0.5 |
| 21 | <0.5 |
| 22 | <0.5 |
| 23 | <0.5 |
| 24 | <2.5 |
| 25 | 0.5 |
| 26 | <12.5 |
| 29 | <2.5 |
| 31 | <2.5 |
| 35 | 0.5 |
| 36 | <0.5 |
| 38 | 2.5 |
| 41 | <0.5 |
| 42 | <12.5 |
| 43 | 0.5 |
| 45 | <2.5 |
| 46 | <0.5 |
| 49 | <2.5 |
| 53 | >12.5 |
| 59 | <0.5 |
| 60 | <2.5 |
| 62 | <0.5 |
| 63 | 2.5 |
| 65 | <0.5 |
| 66 | <0.5 |
| 67 | <0.5 |
| 68 | <0.5 |
| 69 | <0.5 |
| 70 | <2.5 |
| 71 | <2.5 |
| 72 | <12.5 |
| 73 | 0.5 |
| 74 | <0.5 |
| 75 | <2.5 |
| 76 | <2.5 |
| 77 | <2.5 |
| 78 | <2.5 |
| 79 | <12.5 |
| 80 | <2.5 |
| 81 | <2.5 |
| 83 | <12.5 |
| 84 | <12.5 |
| 85 | 12.5 |
| 86 | <0.5 |
| 88 | 12.5 |
| 89 | 12.5 |
| 90 | 12.5 |
| 91 | >12.5 |
| 92 | >12.5 |
| 93 | <12.5 |
| 117 | 12.5 |
| 118 | 12.5 |
| 119 | 12.5 |
| 120 | 12.5 |
| 124 | 12.5 |
| 124 | 2.5 |
| 125 | 0.1 |
| 126 | 0.5 |
| 134 | 12.5 |

### Oncopanel

A panel of human tumor cell lines (A172, BFTC-905, COR-L105, DB, FaDu, H9, Hs 294T, MCF7, MDA MB 436, MeWo, MHH-PREB-1, SJSA1-OSA, SW1353, and U2OS) were grown in RPMI 1640, 10% FBS, 2 mM L-alanyl-L-glutamine, 1 mM Na pyruvate. Cells were seeded into 384-well plates and incubated in a humidified atmosphere with 5% CO2 at 37C. After 24 hours of incubation DMSO or compound was added and plates were incubated for 3 days. Then cells were lysed with CellTiter-Glo (Promega) which generates a bioluminescence signal relative to ATP levels and is used as a measurement of viable cells. Bioluminescence was read by a PerkinElmer Envision microplate reader. Bioluminescence intensity was measured by a PerkinElmer Envision microplate reader and transformed to a percent of control (POC) using the formula: POC=(Ix/I0)*100, where Ix is the whole well signal intensity at a given treatment, and I0 is the average intensity of the untreated vehicle wells.

### Lung Cancer Xenograft

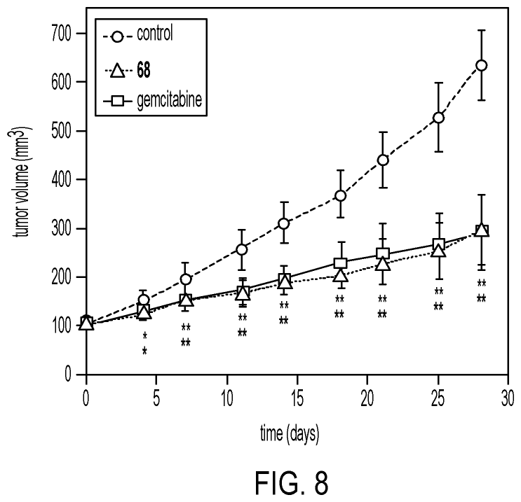

A549 cells growing in RPMI-1640 medium were suspended with Matrigel in PBS. 0.1 ml of cell suspension containing 1×106 cells were injected subcutaneously into the left flank region of female, 6-8 weeks old nude mice (CrTac: Ncr-Foxn1nu). After 30 days of tumor establishment, mice were divided randomly into treatment groups. In the compound 68 study, 6 animals were treated with vehicle only (10% DMSO, 10% propylene glycol, 80% sterile water) by IP once daily, 6 animals were treated with 100 mg/kg gemcitabine hydrochloride by IP twice weekly, and 6 animals were treated with 10 mg/kg of compound 68 by IP once daily for 28 days. Mice were weighed and their tumors were measured using a digital Vernier caliper. Tumor volume was calculated using the formula: (L×W²)/2 where L is the largest diameter and W is the smallest diameter of the tumor. Statistical analysis was performed using Graph Pad Prism (Ver. 5.03). Statistical analysis of tumor growth inhibition between the Control and Treated groups was performed by using One-way ANOVA followed by Dunnett's test. The results are illustrated in FIG. 8.

### Example 16: HIV Screen

MT-2 cells were preseeded in 96-well plates in 100 uL of complete RPMI. Multiple concentrations of PAV-951 were serially diluted in DMSO then into an infection media prepared by diluting NL4-3 Rluc virus stock to 400 IU/100 uL with complete RPMI, which was transferred onto the MT-2 cells with a final MOI of 0.02 and final DMSO concentration of 1% in infected places. One well received DMSO only, instead of PAV-951, and one well received medium only for normalization and background collection. Cells were incubated at 37 degrees Celsius for 96 hours. 100 ul of medium was removed and discarded and 10 uL of 15 uM EnduRen luciferase substrate was added to each well, followed by incubation for 1.5 hours at 37 degrees Celsius. Plates were read on a luminescence plate reader. Bioluminescence intensity was read on a Synergy H1 BioTek plate reader. Averages and standard deviation for viral titer observed under different treatment conditions were calculated in Microsoft Excel and graphed as the percent inhibition in PAV-951 treated cells compared to untreated cells. The results for selected compounds are shown in Table 16.

TABLE 16

| Compound Number | EC₅₀ (uM) |
|---|---|
| 16 | 4.8 |
| 25 | 0.95 |
| 36 | 0.0076 |
| 45 | 0.0031 |
| 46 | 0.003 |
| 57 | 017 |
| 68 | 0.042 |
| 73 | 0.0018 |
| 83 | 0.27 |
| 87 | 10 |
| 92 | 4.3 |
| 97 | 0.23 |
| 98 | 38 |
| 99 | 26 |
| 100 | 33 |

### Example 17: Pox Screen

All compounds mentioned as active were tested on chicken pox. Select hits were also screened against monkey pox.

### Chickenpox

HeLa cells were infected with VACV-LUC virus at MOI 3 in the presence or absence of compound. After 2 h, cells were lysed and luciferase activity was measured as a surrogate for early gene expression. The values were plotted as the percent relative luminescence units (RLU) of each treatment with respect to cells infected with virus alone. The results for selected compounds are shown in Table 17.

TABLE 17

| Compound Number | EC₅₀ (uM) |
|---|---|
| 36 | <1.67 |
| 45 | <1.67 |
| 59 | 0.56 |
| 66 | 1.67 |
| 68 | <0.56 |
| 69 | <5 |
| 73 | 1.67 |
| 77 | 1.67 |
| 83 | 1.67 |

### Monkeypox

BSC-40 cells of 95% confluence in 24-well plates were infected with 100 pfu of MPXV Zaire 79 diluted in Eagle's Minimum Essential Medium with 2% fetal bovine serum and incubated in 37 degrees Celsius in 5% CO₂ for 1 hour. The viral inocula were removed and replaced with the test compounds in six half log dilutions (0.1 ml per well) and the cells were overlaid with 1% methylcellulose in growth media (1 ml per well). The media and virus control cells received growth medium containing 1% methylcellulose. After three days of infection, when plaques appeared, cells were stained with crystal violet for an hour and then washed with water and dried overnight. The plaques were counted the next day and virus-only wells were compared with the compound-added wells to determine percentage protection. Infected cells were stained with crystal violet and viral plaques were counted.

### Example 18: Rabies Screen

The BSR cells [a clone of baby hamster kidney (BHK) cells] were grown in DMEM supplemented with 10% FBS (Atlanta Biologicals) at 37° C. in a 5% CO₂ incubator. The RABV ERA strain was obtained from The American Type Culture Collection and maintained at The Centers for Disease Control and Prevention in Atlanta. For virus titration and antiviral compound treatment, the confluent BSR cells in T75 flasks were split and seeded to a 24-well plate (Fisher Scientific). After 24 h incubation, the confluent BSR cells in the plate were infected with 1 MOI of RABV ERA either before or after treatment with the antiviral compound at the indicated time course. The virus titer in the treated cell supernatants was calculated in focus forming units (ffu) per milliliter. In brief, 20 μL of cell supernatants mixed with 180 μL of freshly prepared BSR cell suspension was seeded into a Lab-Tek Chamber Slide (Fisher Scientific). A serial 10-fold dilution of the virus-cell supernatants was made with similar BSR cell suspensions in the same slide. The cells were incubated at 37° C. in a 5% CO2 incubator for 24 h before titration using the direct fluorescent antigen (DFA) assay. A standard DFA protocol (www.cdc.gov/rabies/pdf/rabiesdfaspv2.pdf) was followed for virus titration for the effect of antiviral compound treatment against the original cells grown in the 24-well plate. The results for selected compounds are shown in Table 18.

TABLE 18

| Compound Number | EC₅₀ (uM) |
|---|---|
| 46 | <0.005 |
| 73 | <0.5 |
| 83 | <0.5 |

### Example 19: Alzheimer's Screen

For viability analysis 4×104 Vero or 8×104 SH-SY5Y-tau P301S cells were plated in a 96-well plate and incubated at 37° C., 5% CO2 overnight. Then the compounds diluted in DMSO were added to the cells. The final concentration of DMSO was 1%. The cells were then incubated for 24 h (Vero) or 48 h (SH-SY5Y). Afterwards 20 μL of a Thiazolyl Blue Tetrazolium Bromide (MTT) solution (5 mg/mL in PBS) was added to the cells and incubated for 4 h allowing viable cells to reduce the yellow MTT into blue formazan metabolites. Following aspiration of the medium, the formazan was resuspended in 200 μL isopropanol/40 mM HCl and incubated for 30 min at RT. The diluted formazan was spectrometrically analyzed at 560 nm and background at 670 nm was subtracted. The results for selected compounds are shown in Table 19.

TABLE 19

| Compound Number | EC⁵⁰ (uM) |
|---|---|
| 16 | 2.52 |
| 45 | 0.02 |
| 87 | 0.43 |
| 92 | 1.86 |
| 94 | 0.45 |
| 95 | 1.82 |
| 96 | 2.12 |
| 97 | 092 |
| 98 | 8.28 |
| 99 | 4.33 |
| 100 | 0.45 |

### Example 20: Antifungal Assay

Test organism C. albicans ATCC 24433 was obtained from American Type Culture collection and maintained at −80° C. The MIC assay method followed the procedure described by clinical and laboratory standards institutes. In this method, an aliquot of 10 mM stock test compound from an 8-point series of 2-fold serial dilutions of compounds in 100% DMSO was added to wells in micro dilution plates.

Inoculum of Candida was prepared as per CLSI methods, colonies picked from streak plate and suspended in RPMI and dilution were made to reach the concentration of cells as described in CLSI methodology. Following inoculation of the microdilution plates, the plates were incubated at 37° C. for 16-20 hr then scored for fungal growth. The MIC is defined as the lowest concentration of test compound that completely inhibits the visible growth of the test organism or a redox fluorescent dye resazurin was added after incubation, fungal growth in wells will convert resazurin into resorufin. The amount of growth in the wells containing the test compound was compared with the amount of growth in the growth-control wells (no test compound used) in each plate, and with the amount of growth in the wells containing control drug (such as Fluconazole).

The results are shown in Table 20

| Compound # | MIC (uM)) |
|---|---|
| 111 | 1.6 |
| 117 | 1.6 |
| 118 | 3.1 |
| 119 | 6.25 |
| 120 | 100 |
| 121 | 1.6 |
| 122 | 3.1 |
| 123 | 3.1 |
| 124 | 1.6 |
| 125 | 6.25 |
| 126 | 12.5 |
| 127 | >100 |
| 128 | 100 |
| 129 | >100 |
| 130 | 6.25 |
| 132 | 12.5 |
| 133 | 100 |
| 134 | >100 |
| 135 | >100 |
| 136 | >100 |
| 137 | >100 |
| 139 | >100 |
| 140 | >100 |
| 141 | >100 |
| 142 | >100 |
| 143 | >100 |
| 145 | 1.6 |
| 146 | 6.25 |
| 147 | 3.1 |
| 148 | 100 |
| 150 | 1.6 |

What is claimed is:

1. A compound of Formula (I): 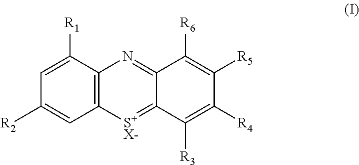
or pharmaceutically acceptable salts, solvates or hydrates thereof wherein:
R₁ is H, —NR₇R₈, —OR₉, alkyl, substituted alkyl, alkenyl, substituted alkenyl, alkynyl, substituted alkynyl, heteroalkyl, substituted heteroalkyl or halo;
R₂ is —H, —NR₁₀R₁₁ or 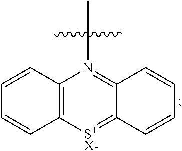
R₃ is —H, halo, —OR₁₂, alkyl or substituted alkyl;
R₄ is —H, —NR₁₃R₁₄ or 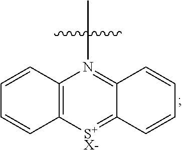
R₅ is —H or halo, —OR₁₅, alkyl or substituted alkyl;
R₆ is —H, —NR₁₆R₁₇, —OR₁₈, alkyl, substituted alkyl, alkenyl, substituted alkenyl, alkynyl, substituted alkynyl, heteroalkyl, substituted heteroalkyl or halo;
R₇ and R₈ are independently alkyl, substituted alkyl, alkenyl, substituted alkenyl, alkynyl, substituted alkynyl, heteroalkyl, substituted heteroalkyl or taken together with the atom to which they are bonded form a cycloheteroalkyl, substituted cycloheteroalkyl, cycloheteroalkenyl, substituted cycloheteroalkenyl, bicycloheteroalkyl, substituted bicycloheteroalkyl, spirocycloheteroalkyl or a substituted spirocycloheteroalkyl ring;
R₁₀ and R₁₁ are independently alkyl, substituted alkyl, alkenyl, substituted alkenyl, alkynyl, substituted alkynyl, heteroalkyl, substituted heteroalkyl or taken together with the atom to which they are bonded form a cycloheteroalkyl, substituted cycloheteroalkyl, cycloheteroalkenyl, substituted cycloheteroalkenyl, bicycloheteroalkyl, substituted bicycloheteroalkyl, spirocycloheteroalkyl or a substituted spirocycloheteroalkyl ring;
R₁₆ and R₁₇ are independently alkyl, substituted alkyl, alkenyl, substituted alkenyl, alkynyl, substituted alkynyl, heteroalkyl, substituted heteroalkyl or taken together with the atom to which they are bonded form a cycloheteroalkyl, substituted cycloheteroalkyl, cycloheteroalkenyl, substituted cycloheteroalkenyl, bicycloheteroalkyl, substituted bicycloheteroalkyl, spirocycloheteroalkyl or a substituted spirocycloheteroalkyl ring;
R₉, R₁₂, R₁₅ and R₁₈ are independently alkyl, substituted alkyl, alkenyl, substituted alkenyl, alkynyl, substituted alkynyl, aryl, substituted aryl, heteroaryl, substituted aryl, heteroalkyl or substituted heteroalkyl;
X— is a pharmaceutically acceptable salt;
provided that when R₂ and R₄ both form a C₅-C₇ cycloheteroalkyl or C₅-C₇ substituted cycloheteroalkyl ring that R₁ is not H when R₆ is t-butyl, R₁ is not H or methyl when R₆ is H except when R₅ is —CH₂NHBoc or —OMe, R₁ is not methyl or ethyl when R₆ is ethyl; provided that none of R₁₀, R₁₁, R₁₃ or R₁₄ are alkyl or substituted alkyl when R₁ and R₆ are independently —H, alkyl, alkenyl or haloalkyl if either R₁₀ and R₁₁ or R₁₃ and R₁₄ form a C₅-C₇ cycloheteroalkyl or C₅-C₇ substituted cycloheteroalkyl ring.

2. The compound of claim 1, wherein R₂ is —NR₉R₁₀ and R₄ is —NR₁₁R₁₂.

3. The compound of claim 2, wherein R₉ and R₁₀ taken together with the atom to which they are bonded form a bicycloheteroalkyl or substituted bicycloheteroalkyl ring and R₁₁ and R₁₂ taken together with the atom to which they are bonded form a bicycloheteroalkyl or substituted bicycloheteroalkyl ring.

4. The compound of claim 3, wherein R₁, R₃, R₅ and R₆ are —H.

5. The compound of claim 3, wherein R₃, R₅ and R₆ are —H.

6. The compound of claim 3, wherein R₁, R₃ and R₆ are —H.

7. The compound of claim 3, wherein R₃ and R₅ are —H.

8. The compound of claim 3, wherein R₅ is —H.

9. The compound of claim 3, wherein R₁, R₃ and R₆ are —H.

10. The compound of claim 2, wherein R₉ and R₁₀ taken together with the atom to which they are bonded form a cycloheteroalkyl, substituted cycloheteroalkyl, cycloheteroalkenyl or substituted cycloheteroalkenyl ring and R₁₁ and R₁₂ taken together with the atom to which they are bonded form a cycloheteroalkyl, substituted cycloheteroalkyl, cycloheteroalkenyl or substituted cycloheteroalkenyl ring.

11. The compound of claim 2, wherein R₉ and R₁₀ taken together with the atom to which they are bonded form a bicycloheteroalkyl or substituted bicycloheteroalkyl ring and R₁₁ and R₁₂ are alkyl or taken together with the atom to which they are bonded form a cycloheteroalkyl, substituted cycloheteroalkyl, cycloheteroalkenyl or substituted cycloheteroalkenyl ring.

12. The compound of claim 2, wherein R₉ and R₁₀ taken together with the atom to which they are bonded form a cycloheteroalkyl, substituted cycloheteroalkyl, cycloheteroalkenyl or substituted cycloheteroalkenyl ring and R₁₁ and R₁₂ are —H.

13. A pharmaceutical composition comprising the compound of claim 1 and a pharmaceutically acceptable excipient.

14. A method of preventing or treating a bacterial infection, cancer, Alzheimer's disease or pox in a patient comprising administering to a patient in need thereof a therapeutically acceptable amount of a compound of claim 1.

15. A method of preventing or treating a bacterial infection, cancer, Alzheimer's disease or pox in a patient comprising administering to a patient in need thereof a therapeutically acceptable amount of a pharmaceutical composition of claim 14.

16. A compound having the having the structure: 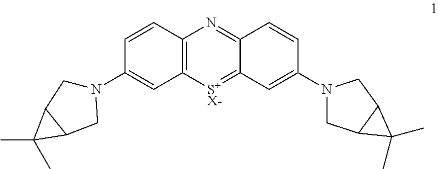
or pharmaceutically acceptable salts, solvates or hydrates thereof.

17. A pharmaceutical composition comprising the compound of claim 16 and a pharmaceutically acceptable excipient.

18. A compound having the having the structure: 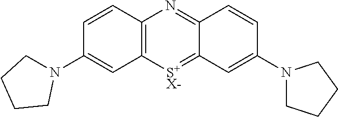
or pharmaceutically acceptable salts, solvates or hydrates thereof.

19. A pharmaceutical composition comprising the compound of claim 18 and a pharmaceutically acceptable excipient.

20. A method of preventing or treating a bacterial infection, cancer, Alzheimer's disease or pox in a patient comprising administering to a patient in need thereof a therapeutically acceptable amount of a compound of claim 16.
